SEQUENCE LISTING (33 sequences)

SEQ ID NO: 1 - DNA, 1821 bp, Artificial Sequence
  Description of Artificial Sequence Synthetic polynucleotide
gaattcatgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtaagcttggagaaacgcgccccagcttctgctgttccagctaccattccattgaccatcaccaacgactctggcagaggtccaatctacctgtacgttttgggtgagagagatggcgttgctggttgggctgatgctggtggtacttttcatccatggccaggtggtgtgggtccagttccagttcctgctccagacgcatctattgctggtccaggtccaggacagtctgttaccattagactgccaaagctgtcgggcagagtgtactactcttacggtcagaagatgaccttccagatcgtgctggacggaagattggttcagccagctgttcagaacgactcggacccaaacagaaacatcctgttcaactggaccgagtacaccttgaacgatggtggtctgtggatcaactctacccaagttgaccattggtctgccccataccaggttggtgttcaacgcgctgatggtcaggttttgtctaccggtatgctgaagccaaacggttacgaggctttctacaccgctttggaatctgctggatggggtggtttggttcaaagagcaccagacggttctagactgagagcattgaacccatctcacggtatcgacgtgggcaagatttcttctgcctcgatcgactcttacgtgaccgaggtttggaactcgtacagaaccagagacatgtgcgtgaccccattttctcacgagccaggtactcagttcagaggtagagtggatggtgactggttcagattcagaaacggctctggtcaagaggttgccgcttttaagaaaccagacgcctcttctgtgtacggctgtcacaaagatcttcaggctccaaacgaccacgttgtgggaccaattgctagaaccttgtgtgccgctctggttagaaccactgctctgactaacccaaaccagccagatgctaactctgccggtttttaccaggacgccagaactaacgtttacgccaaattggctcaccagcagatggccaacggtaaggcttacgcttttgctttcgacgacgttggtgctcacgagtctttggttcatgacggtaatccacaggccgcctacattaagttggacccattcactggtactgctaccccaattgcaaacggtggttctactgagcaacctggaactccaggtggtttgccagctggtactggtgctttgagaattggttctaccctgtgtctggacgttccatgggctgatccaactgacactaaccaggttcagctggctacctgttctggtaacgctgctcaacaatggaccagaggtactgacggtactgttagagccctgggaaagtgtttggacgtggctagatctggaactgctgatggtacagccgtgtggatctacacctgtaatggaactggtgctcagaagtggacctacgactctgctactaaggctctgagaaacccacagtctggtaagtgcttggatgctcaaggtggtgctccattgagagatggtcagaaagttcagctgtggacctgcaaccagactgaggctcaaagatggaccctgtaaggatcc

SEQ ID NO: 2 - DNA, 1476 bp, Artificial Sequence
  Description of Artificial Sequence Synthetic polynucleotide
caattgatgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtaagcttggagaaacgcaccccagtgtctaccaacgacgtgtcggttgagaaaagagcctctggttacaccaacgccgtgtactttacaaactggggcatctacggcagaaacttccagccacaagacttggttgcctctgacatcacccacgtgatctacccattcatgaacttccaggctgacggaaccgttgtttctggtgatgcttacgctgactaccagaagcactactcggacgactcttggaacgacgttggtaacaacgcctacggttgtgtgaaccagctgttcaagctgaagaaggccaacagaaacctgaaggtgatgctgtctattggcggttggacttggtcgactaacttcccatctgctgcttctaccgacgctaacagaaagaacttcgccaagaccgccatcacgttcatgaaggattggggtttcgacggtatcgacgttgactgggaatacccagctgacgatactcaggctaccaacatggttctgctgctgaaagagatcagatcgcagttggacgcttacgcagctcaatacgctccaggttaccacttcctgttgtctattgctgctcctgctggtccagagcattactctgcattgcacatggctgaccttggtcaggttctggactacgttaacctgatggcttacgactacgctggctcttggtcgtcttactctggtcacgatgctaacctgttcgctaacccatcgaacccaaactcgtcgccatacaacactgaccaggccatcaaggcctacatcaacggtggtgttccagcctctaagatcgttctgggtatgccaatctacggtagatcgttcgagtccaccaacggtattggtcagacctacaacggtatcggctctggttcttgggagaacggtatttgggactacaaggtgttgccaaaggctggtgctactgtgcagtacgactctgttgctcaggcctactactcttacgactcgtcgtctaaagagctgatctcgtttgacaccccagacatggtgtcgaagaaggtgtcttacctgaagaaccttggccttggcggttcgatgttttgggaagcttctgctgacaagaccggctctgactctctgattggtacttctcacagagccctgggttctttggactctactcagaacttgctgtcgtaccctaacagccagtacgacaacatcagatcgggcctgaactaaagatct

SEQ ID NO: 3 - protein, 125 aa, Artificial Sequence
  Description of Artificial Sequence Synthetic polypeptide
MKFSFVYGLTGFLAATSSALPSEILSTGYERSALEKRGDGYLIMCKNCDPNTGSCDWKQNWNTCVGIGANVHWMVTGGSTDGKQGCATIWEGSGCVGRSTTMCCPANTCCNINTGFYIRSYRRVE

SEQ ID NO: 4 - DNA, 534 bp, Artificial Sequence
  Description of Artificial Sequence Synthetic polynucleotide
gaattcatgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtaagcttggagaaacgcggtgacggttacctgatcatgtgcaagaactgtgacccaaacaccggttcttgtgactggaagcagaactggaacacctgtgttggtatcggtgctaacgtgcactggatggttactggtggttctactgacggtaagcagggttgtgctaccatctgggaaggttctggttgtgtgggaagatctaccaccatgtgctgtccagctaacacttgctgcaacatcaacaccggcttctacatcagatcgtacagaagagtcgagtaaggatcc

SEQ ID NO: 5 - protein, 644 aa, Artificial Sequence
  Description of Artificial Sequence Synthetic polypeptide
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTHNPPIPVGEIYKRWIILGLNKIVRMYSPTSILDIRQGPKEPFRDYVDRFYKTLRAEQASQEVKNWMTETLLVQNANPDCKTILKALGPGATLEEMMTACQGVGGPGHKARVLAEAMSQVTNPATIMIQKGNFRNQRKTVKCFNCGKEGHIAKNCRAPRKKGCWKCGKEGHQMKDCTERQANFLGKIWPSHKGRPGNFLQSRPEPTAPPEESFRFGEETTTPSQKQEPIDKELYPLASLRSLFGSDPSSQGGSTSSEFCSRRYRGPGIHRPVATMASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYAAA

SEQ ID NO: 6 - DNA, 587 bp, Artificial Sequence
  Description of Artificial Sequence Synthetic polynucleotide
cccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaacc

SEQ ID NO: 7 - protein, 548 aa, Artificial Sequence
  Description of Artificial Sequence Synthetic polypeptide
MPHDRKNSSRRAWAALCAAVLAVSGALVGVAAPASAVPATIPLTITNDSGRGPIYLYVLGERDGVAGWADAGGTFHPWPGGVGPVPVPAPDASIAGPGPGQSVTIRLPKLSGRVYYSYGQKMTFQIVLDGRLVQPAVQNDSDPNRNILFNWTEYTLNDGGLWINSTQVDHWSAPYQVGVQRADGQVLSTGMLKPNGYEAFYTALESAGWGGLVQRAPDGSRLRALNPSHGIDVGKISSASIDSYVTEVWNSYRTRDMCVTPFSHEPGTQFRGRVDGDWFRFRNGSGQEVAAFKKPDASSVYGCHKDLQAPNDHVVGPIARTLCAALVRTTALTNPNQPDANSAGFYQDARTNVYAKLAHQQMANGKAYAFAFDDVGAHESLVHDGNPQAAYIKLDPFTGTATPIANGGSTEQPGTPGGLPAGTGALRIGSTLCLDVPWADPTDTNQVQLATCSGNAAQQWTRGTDGTVRALGKCLDVARSGTADGTAVWIYTCNGTGAQKWTYDSATKALRNPQSGKCLDAQGGAPLRDGQKVQLWTCNQTEAQRWTL

SEQ ID NO: 8 - protein, 337 aa, Artificial Sequence
  Description of Artificial Sequence Synthetic polypeptide
AHTVSPVNPNAQQTTKTVMNWLAHLPNRTENRVLSGAFGGYSHDTFSMAEADRIRSATGQSPAIYGCDYARGWLETANIEDSIDVSCNGDLMSYWKNGGIPQISLHLANPAFQSGHFKTPITNDQYKKILDSSTAEGKRLNAMLSKIADGLQELENQGVPVLFRPLHEMNGEWFWWGLTSYNQKDNERISLYKQLYKKIYHYMTDTRGLDHLIWVYSPDANRDFKTDFYPGASYVDIVGLDAYFQDAYSINGYDQLTALNKPFAFTEVGPQTANGSFDYSLFINAIKQKYPKTIYFLAWNDEWSPAVNKGASALYHDSWTLNKGEIWNGDSLTPIVE

SEQ ID NO: 9 - protein, 487 aa, Artificial Sequence
  Description of Artificial Sequence Synthetic polypeptide
MRFPSIFTAVLFAASSALAAPVNTTTEDETAQIPAEAVIGYSDLEGDFDVAVLPFSNSTNNGLLFINTTIASIAAKEEGVSLEKRTPVSTNDVSVEKRASGYTNAVYFTNWGIYGRNFQPQDLVASDITHVIYPFMNFQADGTVVSGDAYADYQKHYSDDSWNDVGNNAYGCVNQLFKLKKANRNLKVMLSIGGWTWSTNFPSAASTDANRKNFAKTAITFMKDWGFDGIDVDWEYPADDTQATNMVLLLKEIRSQLDAYAAQYAPGYHFLLSIAAPAGPEHYSALHMADLGQVLDYVNLMAYDYAGSWSSYSGHDANLFANPSNPNSSPYNTDQAIKAYINGGVPASKIVLGMPIYGRSFESTNGIGQTYNGIGSGSWENGIWDYKVLPKAGATVQYDSVAQAYYSYDSSSKELISFDTPDMVSKKVSYLKNLGLGGSMFWEASADKTGSDSLIGTSHRALGSLDSTQNLLSYPNSQYDNIRSGLN

SEQ ID NO: 10 - DNA, 1935 bp, Artificial Sequence
  Description of Artificial Sequence Synthetic polynucleotide
atgggtgctagagcttctgttttgtctggtggtgaattggatagatgggaaaagatcagattaagaccaggtggtaagaagaagtacaagttgaaacatatcgtttgggcctccagagaattggaaagatttgctgttaatccaggcttgttggaaacttctgaaggttgcagacaaatcttgggtcaattgcaaccatctttacaaaccggttctgaagaactgagatccttgtataatactgttgctaccttgtactgcgtccatcaaagaattgaaatcaaggataccaaagaggccttggacaagattgaagaagaacagaacaagtccaagaagaaggctcaacaagctgctgctgatactggtcattctaatcaagtttctcagaactacccaatcgtccaaaacattcaaggtcaaatggttcatcaagccatttctccaagaactttgaatgcatgggtcaaagttgtagaagaaaaggctttttcacctgaggttatcccaatgttttctgctttatctgaaggtgctactccacaagatttgaacactatgttgaacactgttggtggtcatcaagctgctatgcaaatgttgaaagaaaccattaacgaagaggctgctgaatgggatagagttcatccagttcatgctggtccaattgcacctggtcaaatgagagaacctagaggttctgatattgctggtactacttctaccttgcaagaacaaattggttggatgactcataacccaccaattccagttggtgaaatctacaagagatggatcattttgggcttgaacaagatcgtcagaatgtactctccaacctccattttggatattagacagggtccaaaagaaccattcagagattacgttgacagattctacaagaccttgagagctgaacaagcttctcaagaggttaagaattggatgaccgaaaccttgttggttcaaaatgctaatccagattgcaagaccatcttgaaagctttaggtccaggtgctacattggaagaaatgatgactgcttgtcaaggtgtaggtggtccaggtcataaggctagagttttggctgaagctatgtctcaagttactaatccagccaccattatgatccaaaagggcaactttagaaaccagagaaagaccgttaagtgcttcaactgtggtaaagaaggtcatattgctaagaactgtagagccccaagaaaaaaaggttgttggaagtgcggaaaagagggtcatcaaatgaaggattgcactgaaagacaagctaacttcttgggtaaaatttggccatctcataagggtagaccaggtaactttttacaatctagaccagaacctactgctccaccagaagaatcttttagatttggtgaagaaaccaccactccatctcaaaagcaagaacctatcgacaaagagttgtatccattggcttcattgagatctttgttcggttctgatccatcatctcaaggtggttctacatcttctgaattctgctctagaagatatagaggtcctggtattcatagaccagttgctactatggcttctaacttcactcaattcgttttggttgataacggtggtactggtgatgttacagttgctccatctaatttcgctaacggtattgcagaatggatctcctctaattctagatcccaagcttacaaggttacctgctctgttagacaatcttctgctcagaatagaaagtacaccatcaaggttgaagttccaaaaggtgcttggagatcttacttgaacatggaattgaccattccaattttcgccaccaactctgattgtgaattgatcgttaaggctatgcagggtttgttgaaggatggtaatccaattccatctgctattgctgctaattctggtatctatgctgctgcttaa

SEQ ID NO: 11 - DNA, 1394 bp, Artificial Sequence
  Description of Artificial Sequence Synthetic polynucleotide
gtcgacatggtgtctaaaggtgaagagttgttcactggtgttgttccaattttggttgaattggatggtgatgttaacggccataagttttctgtttctggtgaaggtgagggtgatgctacttatggtaaattgactttgaagttcatctgcaccacaggtaaattgccagttccatggccaactttggttactactttgacttatggtgtccagtgcttttctagatacccagatcatatgaagcaacacgactttttcaaatccgctatgccagaaggttacgttcaagaaagaaccatcttcttcaaggatgacggtaactacaaaactagagccgaagttaagttcgaaggtgataccttggttaacaggattgaattgaagggcatcgattttaaagaggacggtaacattttgggtcacaagttggagtacaactacaactctcataacgtttacatcatggccgacaagcaaaagaacggtattaaggttaacttcaagatcaggcacaacatcgaagatggtagtgttcaattggctgatcactaccaacaaaacactccaattggtgatggtccagttttgttgccagataaccattacttgtctacccaatctgctttgtctaaggacccaaacgaaaaaagggatcacatggtcttgttggaatttgttactgctgctggtattaccttaggtatggacgaattatacaagtaaggatcctaaggtacctaattgcctagaaaacatgaggatcacccatgtctgcaggtcgactctagaaaacatgaggatcacccatgtctgcagtattcccgggttcattagatcctaaggtacctaattgcctagaaaacatgaggatcacccatgtctgcaggtcgactctagaaaacatgaggatcacccatgtctgcagtattcccgggttcattagatcctaaggtacctaattgcctagaaaacatgaggatcacccatgtctgcaggtcgactccagaaaacatgaggatcacccatgtctgcagtattcccgggttcattagatcctaaggtacctaattgcctagaaaacatgaggatcacccatgtctgcaggtcgactctagaaaacatgaggatcacccatgtctgcagtattcccgggttcattagatcctaaggtacctaattgcctagaaaacatgaggatcacccatgtctgcaggtcgactctagaaaacatgaggatcacccatgtctgcagtattcccgggttcattagatcctaaggtacctaattgcctagaaaacatgaggatcacccatgtctgcaggtcgactccagaaaacatgaggatcacccatgtctgcagtattcccgggttcattagatctgcggccgc

SEQ ID NO: 12 - RNA, 19 nt, Artificial Sequence
  Description of Artificial Sequence Synthetic oligonucleotide
  modified_base (1)..(1): a, c, u, g, unknown or other
  modified_base (3)..(3): a, c, u, g, unknown or other
  modified_base (10)..(10): a, c, u, g, unknown or other
  modified_base (16)..(17): a, c, u, g, unknown or other
  modified_base (19)..(19): a, c, u, g, unknown or other
nrndsassancasssnnyn

SEQ ID NO: 13 - DNA, 4011 bp, Artificial Sequence
  Description of Artificial Sequence Synthetic polynucleotide
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagatcccaataccggtaacctacaaacgggtggaggatcaccccacccgacacttcacaatcaaggggtacaatacacaagggtggaggaacaccccaccctccagacacattacacagaaatccaatcaaacagaagcaccatcagggcttctgctaccaaatttatctcaaaaaactacaacaaggaatcaccatcagggattccctgtgcaatatacgtcaaacgagggccacgacgggaggacgatcacgcctcccgaatatcggcatgtctggctttcgaattcagtgcgtggagcatcagcccacgcagccaatcagagtcgaatacaagtcgactttcgcgaagagcatcagccttcgcgccattcttacacaaaccacactctcccctacaggaacagcatcagcgttcctgcccagtacccaactcaagaaaatttatgtccccatgcagcatcagcgcatgggccccaagaatacatccccaacaaaatcacatccgagcaccaacagggctcggagtgttgtttcttgtccaactggacaaaccctccatggaccatcaggccatggactctcaccaacaagacaaaaactactcttctcgaagcagcatcagcgcttcgaaacactcgagcatacattgtgcctatttcttgggtggacgatcacgccacccatgctctcacgaatttcaaaacacggacaaggacgagcaccaccagggctcgtcgttccacgtccaatacgattacttacctttcgggatcacgatcacggatcccgcagctacatcacttccactcaggacattcaagcatgcacgatcacggcatgctccacaagtctcaaccacagaaactaccaaatgggttcagcaccagcgaacccactcctacctcaaacctcttcccacaaaactggcaagcaggatcaccgcttgcccattccaacataccaaatcaaaaacaattactggtacagcatcagcgtaccagcccacatctctcactactatcaaaaaccaaaccgttcagcaacagcgaacggtacacacggaaaaatcaactggtttacaaatacgaaagacgatcacgctttcgtccagcgcaaactattacgaaaaacatccgacgggaagagcaacagccttcccgcggcggaaaacctcacaaaaacacgacaaacggatgcacgaacacggcatccgccgacaacccacaaacttacaaccaggcaaacggtgcaggatcaccgcaccgtacatcaaacacctcagatctcatatcgatattgggtcatcggatcccgggcccgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 14 - protein, 551 aa, Artificial Sequence
  Description of Artificial Sequence Synthetic polypeptide
MGIIIQFLVVSCLLAGAGGLDPAALMQIGVIPTNVRQLMYYTEASSAFIVVKLMPTIDSPISGCNITSISSYNATVTKLLQPIGENLETIRNQLIPTRRRRRFAGVVIGLAALGVATAAQVTAAVALVKANENAAAILNLKNAIQKTNAAVADVVQATQSLGTAVQAVQDHINSVVSPAITAANCKAQDAIIGSILNLYLTELTTIFHNQITNPALSPITIQALRILLGSTLPTVVEKSFNTQISAAELLSSGLLTGQIVGLDLTYMQMVIKIELPTLTVQPATQIIDLATISAFINNQEVMAQLPTRVMVTGSLIQAYPASQCTITPNTVYCRYNDAQVLSDDTMACLQGNLTRCTFSPVVGSFFTRFVLFDGIVYANCRSMLCKCMQPAAVILQPSSSPVTVIDMYKCVSLQLDNLRFTITQLANVTYNSTIKLESSQILPIDPLDISQNLAAVNKSLSDALQHLAQSDTYLSAITSATTTSVLSIIAICLGSLGFILIILLSVVVWKLLTIVAANRNRMENFVYHNQAFHHSQSDLSEKNQPATLGTR

SEQ ID NO: 15 - protein, 2255 aa, Artificial Sequence
  Description of Artificial Sequence Synthetic polypeptide
MAGSREILLPEVHLNSPIVKHKLYYYILLGNLPNEIDLDDLGPLHNQNWNQIAHEESNLAQRLVNVRNFLITHIPDLRKGHWQEYVNVILWPRILPLIPDFKINDQLPLLKNWDKLVKESCSVINAGTSQCIQNLSYGLTGRGNLFTRSRELSGDRRDIDLKTVVAAWHDSDWKRISDFWIMIKFQMRQLIVRQTDHNDPDLITYIENREGIIIITPELVALFNTENHTLTYMTFEIVLMISDMYEGRHNILSLCTVSTYLNPLKKRITYLLSLVDNLAFQIGDAVYNIIALLESFVYAQLQMSDPIPELRGQFHAFVCSEILDALRGTNSFTQDELRTVTTNLISPFQDLTPDLTAELLCIMRLWGHPMLTASQAAGKVRESMCAGKVLDFSTIMKTLAFFHTILINGYRRKHHGVWPPLNLPGNASKGLTELMNDNTEISYEFTLKHWKEISLIKFKKCFDADAGEELSIFMKDKAISAPKQDWMSVFRRSLIKQRHQHHQVPLPNPFNRRLLLNFLEDDKFDPNVELQYVTSGEYLHDDTFCASYSLKEKEIKPDGRIFAKLTKRMRSCQVIAESLLANHAGKLMKENGVVMNQLSLTKSLLTMSQIGIISEKARKSTRDNINQPGFQNIQRNKSHHSKQVNQRDPSDDFELAASFLTTDLKKYCLQWRYQTIIPFAQSLNRMYGYPHLFEWIHLRLMRSTLYVGDPFNPPADTSQFDLDKVINGDIFIVSPRGGIEGLCQKAWTMISISVIILSATESGTRVMSMVQGDNQAIAVTTRVPRSLPTLEKKTIAFRSCNLFFERLKCNNFGLGHHLKEQETIISSHFFVYSKRIFYQGRILTQALKNASKLCLTADVLGECTQSSCSNLATTVMRLTENGVEKDICFYLNIYMTIKQLSYDIIFPQVSIPGDQITLEYINNPHLVSRLALLPSQLGGLNYLSCSRLFNRNIGDPVVSAVADLKRLIKSGCMDYWILYNLLGRKPGNGSWATLAADPYSINIEYQYPPTTALKRHTQQALMELSTNPMLRGIFSDNAQAEENNLARFLLDREVVFPRVAHIIIEQTSVGRRKQIQGYLDSTRSIMRKSLESKPLSNRKLNEILDYNINYLAYNLALLKNAIEPPTYLKAMTLETCSIDIARSLRKLSWAPLLGGRNLEGLETPDPIEITAGALIVGSSYCEQCAAGDNRFTWFFLPSGIEIGGDPRDNPPIRVPYIGSRTDERRVASMAYIRGASSSLKAVLRLAGVYIWAFGDTLENWIDALDLSHTRVNITLEQLQSLTPLPTSANLTHRLDDGTTTLKFTPASSYTFSSFTHISNDEQYLTINDKTADSNIIYQQLMITGLGILETWNNPPINRTFEESTLHLHTGASCCVRPVDSCIISEALTVKPHITVPYSNKFVFDEDPLSEYETAKLESLSFQAQLGNIDAVDMTGKLTLLSQFTARQIINAITGLDESVSLTNDAIVASDYVSNWISECMYTKLDELFMYCGWELLLELSYQMYYLRVVGWSNIVDYSYMILRRIPGAALNNLASTLSHPKLFRRAINLDIVAPLNAPHFASLDYIKMSVDAILWGCKRVINVLSNGGDLELVVTSEDSLILSDRSMNLIARKLTLLSLIHHNGLELPKIKGFSPDEKCFALTEFLRKVVNSGLSSIENLSNFMYNVENPRLAAFASNNYYLTRKLLNSIRDTESGQVAVTSYYESLEYIDSLKLTPHVPGTSCIEDDSLCINDYIIWIIESNANLEKYPIPNSPEDDSNFHNFKLNAPSHHTLRPLGLSSTAWYKGISCCRYLERLKLPQGDHLYIAEGSGASMTIIEYLFPGRKIYYNSLFSSGDNPPQRNYAPMPTQFIESVPYKLWQAHTDQYPEIFEDFIPLWNGNAAMTDIGMTACVEFIINRVGPRTCSLVHVDLESSASLNQQCLSKPIINAIITATTVLCPHGVLILKYSWLPFTRFSTLITFLWCYFERITVLRSTYSDPANHEVYLICILANNFAFQTVSQATGMAMTLTDQGFTLISPERINQYWDGHLKQERIVAEAIDKVVLGENALFNSSDNELILKCGGTPNARNLIDIEPVATFIEFEQLICTMLTTHLKEIIDITRSGTQDYESLLLTPYNLGLLGKISTIVRLLTERILNHTIRNWLILPPSLRMIVKQDLEFGIFRITSIFNSDRFLKLSPNRKYLIAQLTAGYIRKLIEGDCNIDLTRPIQKQIWKALGCVVYCHDPMDQRESTEFIDININEEIDRGIDGEEI

SEQ ID NO: 16 - protein, 509 aa, Artificial Sequence
  Description of Artificial Sequence Synthetic polypeptide
MSSVLKAYERFTLTQELQDQSEEGTIPPTTLKPVIRVFILTSNNPELRSRLLLFCLRIVLSNGARDSHRFGALLTMFSLPSATMLNHVKLADQSPEADIERVEIDGFEEGSFRLIPNARSGMSRGEINAYAALAEDLPDTLNHATPFVDSEVEGTAWDEIETFLDMCYSVLMQAWIVTCKCMTAPDQPAASIEKRLQKYRQQGRINPRYLLQPEARRIIQNVIRKGMVVRHFLTFELQLARAQSLVSNRYYAMVGDVGKYIENCGMGGFFLTLKYALGTRWPTLALAAFSGELTKLKSLMALYQTLGEQARYLALLESPHLMDFAAANYPLLYSYAMGIGYVLDVNMRNYAFSRSYMNKTYFQLGMETARKQQGAVDMRMAEDLGLTQAERTEMANTLAKLTTANRGADTRGGVNPFSSVTGTTQVPASATGDTFEGYMAADRLRQRYADAGTYDDEMPPLEEEKEDDTSAGPRTGPTLEQVALDIQNAAVGAPIHTDDLNAALGDLDI

SEQ ID NO: 17 - protein, 392 aa, Artificial Sequence
  Description of Artificial Sequence Synthetic polypeptide
MDPTDLSFSPDEINKLIETGLNTVEYFTSQQVTGTSSLEKNTIPPGVTGLLTNAAEAKIQESTNHQKGSVGGGAKPKKPRPKIAIVPADDKTVPGKPIPNPLLGLDSTPSTQTVLDLSGKTLPSGSYKGVKLAKFGKENLMTRFIEEPRENPIATSFPIDFKRGAGIPAGSIEGSTQLDGWEMKSRSLSGAIHPVLQSPLQQGDLNALVTSVQSLALNVNEILNTVRNLDSRMNQLETKVDRILSSQSLIQTIKNDIVGLKAGMATLEGMITTVKIMDPGVPSNVTVEDVRKTLSNHAVVVPESFNDSFLTQSEDVISLDELARPTATSVKKIVRKVPPQKDLTGLKITLEQLAKDCISKPKMREEYLLKINQASSEAQLIDLKKAIIRSAI

SEQ ID NO: 18 - DNA, 5464 bp, Artificial Sequence
  Description of Artificial Sequence Synthetic polynucleotide
ggtacccaccagagattggcacccttccagacatatttcgttcgcaaagaaccataaagataaagaaccctattcaacatacacctaccactctccaaggccaggcgatgattccacgcaagagggtattttgtggcccgtacactgtgtgaaaaacacctggggtagtaattggttgaccaaataatggaccaagtggtcactaagcatattaagattgtatacaagggtttcttgactgaccgtgaatactactccgccttccacgacatctggaacttccataagaccgacatgaacaagtacttagaaaagcatcatacagacgaggtttacattgtcggtgtagctttggagtattgtgtcaaagccaccgccatttccgctgcagaactaggttataagaccactgtcctgctggattacacaagacccatcagcgatgatcccgaagtcatcaataaggttaaggaagagttgaaggcccacaacatcaatgtcgtggataaataagagctgaataatacttcttcaacctgatgaactagggtggcttgcaaatgcacaaatctatataacaatatctatatatatgtatgtacaccgaattccgacatatggagaaggaataataaaatattaactaacgtctttacgcctctctttattcttttttgggtaaattgcttaaactatttggccggcccaccgcgaaaagatttggctgggcctcaactaaacgcgcctttttggacttttcacgttgcagggacagcaacgtcaaaacttctgcattaaggtagtttggtagcttggtagccactttagtatttctgccttcttcgaataccgacattatttctcgccaatccacattctctctccccatctgcatccttttatatttaatagggataggttgttttagttctttgattccgttttcatttcaagagcaataatagcaatttggaaaaagaaagcaagtaaaagaaagaagagatcttattttttgctttttctcttgaggtcacatgatcgcaaaatggcaaatggcacgtgaagctgtcgatattggggaactgtggtggttggcaaatgactaattaagttagtcaaggcgccatcctcatgaaaactgtgtaacataataaccgaagtgtcgaaaaggtggcaccttgtccaattgaacacgctcgatgaaaaaaataagatatatataaggttaagtaaagcgtctgttagaaaggaagtttttcctttttcttgctctcttgtcttttcatctactatttccttcgtgtaatacagggtcgtcagatacatagatacaattctattacccccatccatacacccgccgccaccatgccatctcatttcgatactgttcaactacacgccggccaagagaaccctggtgacaatgctcacagatccagagctgtaccaatttacgccaccacttcttatgttttcgaaaactctaagcatggttcgcaattgtttggtctagaagttccaggttacgtctattcccgtttccaaaacccaaccagtaatgttttggaagaaagaattgctgctttagaaggtggtgctgctgctttggctgtttcctccggtcaagccgctcaaacccttgccatccaaggtttggcacacactggtgacaacatcgtttccacttcttacttatacggtggtacttataaccagttcaaaatctcgttcaaaagatttggtatcgaggctagatttgttgaaggtgacaatccagaagaattcgaaaaggtctttgatgaaagaaccaaggctgtttatttggaaaccattggtaatccaaagtacaatgttccggattttgaaaaaattgttgcaattgctcacaaacacggtattccagttgtcgttgacaacacatttggtgccggtggttacttctgtcagccaattaaatacggtgctgatattgtaacacattctgctaccaaatggattggtggtcatggtactactatcggtggtattattgttgactctggtaagttcccatggaaggactacccagaaaagttccctcaattctctcaacctgccgaaggatatcacggtactatctacaatgaagcctacggtaacttggcatacatcgttcatgttagaactgaactattaagagatttgggtccattgatgaacccatttgcctctttcttgctactacaaggtgttgaaacattatctttgagagctgaaagacacggtgaaaatgcattgaagttagccaaatggttagaacaatccccatacgtatcttgggtttcataccctggtttagcatctcattctcatcatgaaaatgctaagaagtatctatctaacggtttcggtggtgtcttatctttcggtgtaaaagacttaccaaatgccgacaaggaaactgacccattcaaactttctggtgctcaagttgttgacaatttaaagcttgcctctaacttggccaatgttggtgatgccaagaccttagtcattgctccatacttcactacccacaaacaattaaatgacaaagaaaagttggcatctggtgttaccaaggacttaattcgtgtctctgttggtatcgaatttattgatgacattattgcagacttccagcaatcttttgaaactgttttcgctggccaaaaaccataataagtgtgcgtaatgagttgtaaaattatgtataaacctccatatccaacttccaatttaatctttcttttttaattttcacttatttgcgatacagaaagaggatccaacggagcagtgcggcgcgaggcacatctgcgtttcaggaacgcgaccggtgaagacgaggacgcacggaggagagtcttccttcggagggctgtcacccgctcggcggcttctaatccgtacttcaatatagcaatgagcagttaagcgtattactgaaagttccaaagagaaggtttttttaggctaagataatggggctctttacatttccacaacatataagtaagattagatatggatatgtatatggatatgtatatggtggtaatgccatgtaatatgattattaaacttctttgcgtccatccaaaaaaaaagtaagaatttttggtcgaccccgccgccaccatgtctaatttgttgactgttcatcaaaatttgccagctttgccagttgatgcaacgtctgatgaggtaagaaagaatttgatggatatgtttagagatagacaagcattctctgaacatacttggaaaatgttgttgtctgtttgtagatcttgggctgcttggtgtaaattaaataatagaaaatggtttccagctgaaccagaagatgttagagattatttgttgtatttgcaagctagaggtttggctgttaaaactatacaacaacatttgggtcaattaaatatgttgcataggagatctggtttgcctagaccatctgattctaatgctgtatctttggttatgagaaggattagaaaagaaaatgttgatgcaggtgaaagagctaaacaagcattggcgtttgaaagaactgattttgatcaagttagatctttgatggaaaattctgatagatgtcaagatataagaaacttggctttcttgggaattgcttataatacattgttgagaattgctgaaattgctagaattagagttaaagacatttctagaactgatggtggtagaatgttgattcatattggtagaactaaaacattggtttcaactgctggcgttgagaaagcattgtctttgggtgttactaaattggttgaaagatggatttctgtatctggtgtggctgatgacccaaataattatttattttgtagagttagaaagaatggtgttgcggctccatctgctacatctcagctctctacccgtgccttggaaggaatttttgaagcaactcatagattgatttatggtgctaaagatgattctggtcaaagatatttagcatggtctggtcattctgctagagttggtgccgctagagatatggctagagctggggtatctattcctgaaattatgcaagctggtggttggactaatgttaacatcgtcatgaactatattagaaatttggattctgaaactggtgcaatggttagactcttggaagatggtgattaataagaccgcgtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcgaattccgaggctctcctttcatcataccctctctaaataaaatttattcctagttattcctttcttcttcatatccttaatcatgcattcactgccatgtgcaaataaaggctcttgtggtagaaacttttagctcaaacgtggtaaaactatctcaacgtccttccgtagacaactggtgagcagtgcttctgtaacactgcagatctccaatgataaatcgataccgtactaactgcccgcactataatgaactctttgtatccgtaactatttaaaatattcattcatttctataagctatatgtttatatttagattagaggggttaaaagaaagtttttctcgaaagcttaattttagcttttactttgaacttctagtaattgcgaggcagtatcgacaatgcaatctctgaacttattaaattggaatcctaataaatttttggttttgcggttgtcgcacttgcagcagttttttgtcaaaaaggttgagccgctaccaggttcgccagttgctatcttgccttttaactgtggaaattcctcattcaagatatccagcgcttcttgagagcaaaacatatcttcacataagaatagtctttggccagcgcattcgggtttctcaaatgcaagtaaatgagcttttgaaacatcgcgaacgtcaataaaaggaccactgtaattataaaaattgtcgcctaatttataactaaccaaattggcaataatggctgaagagctatttattccatttctaagagagtcggcaaatagctgagggccaaaaacaaatcctgggttgatggttgatagcgtaaatttgatgcttgattggttttcctcgagaaaatcccaagcagttttttcagcaaatttcttggaaccacagtatgcggaaaccgcgttagcttgacaactttcccaagtatctttgttccaactttcctcattgacaacgaaactagtgtccttcatatctccgggagatgcaagggcaggtacc

SEQ ID NO: 19 - DNA, 20 bp, Artificial Sequence
  Description of Artificial Sequence Synthetic primer
ttatccgcctccatccagtc

SEQ ID NO: 20 - DNA, 20 bp, Artificial Sequence
  Description of Artificial Sequence Synthetic primer
ctgcggccaacttacttctg

SEQ ID NO: 21 - DNA, 23 bp, Artificial Sequence
  Description of Artificial Sequence Synthetic primer
ccagctttgccagttgatgcaac

SEQ ID NO: 22 - DNA, 25 bp, Artificial Sequence
  Description of Artificial Sequence Synthetic primer
gctctagccatatctctagcggcac

SEQ ID NO: 23 - DNA, 20 bp, Artificial Sequence
  Description of Artificial Sequence Synthetic primer
ggctgggcctcaactaaacg

SEQ ID NO: 24 - DNA, 23 bp, Artificial Sequence
  Description of Artificial Sequence Synthetic primer
cgatgttgtcaccagtgtgtgcc

SEQ ID NO: 25 - DNA, 25 bp, Artificial Sequence
  Description of Artificial Sequence Synthetic primer
gtgccgctagagatatggctagagc

SEQ ID NO: 26 - DNA, 20 bp, Artificial Sequence
  Description of Artificial Sequence Synthetic primer
ctgctgcaagtgcgacaacc

SEQ ID NO: 27 - DNA, 375 bp, Artificial Sequence
  Description of Artificial Sequence Synthetic polynucleotide
caggtgcagctgcaggagtctgggggaggattggtgcaggctgggggctctctgagactctcctgtgccgcctctggacgcaccttcagtgaatatgccatgggctggttccgccaggctccagggaaggaacgtgagtttgtagcaactattagctggagtggtggtagcacatactatacagactccgtgaagggccgattcaccatctccagagacaacgccaagaacacggtgtatctgcaaatgaacagcctgaaacctgatgacacggccgtttattactgtgcagcagccgggttaggtacggtagtatcggagtgggattatgactatgactactggggccaggggacccaggtcaccgtctcctca

SEQ ID NO: 28 - DNA, 339 bp, Artificial Sequence
  Description of Artificial Sequence Synthetic polynucleotide
gacatccagttgacccagtctccagactccctggctgtgtctctgggcgagagggccaccatcaactgcaagtccagccagagtgttttatacagctccatcaataagaactacttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatattatagtactccgtacacttttggccaggggaccaaggtggaaatcaaa

SEQ ID NO: 29 - DNA, 351 bp, Artificial Sequence
  Description of Artificial Sequence Synthetic polynucleotide
cagatgcagctggtgcaatctggaacagaggtgaaaaagccgggggagtctctgaagatctcctgtaagggttctggatacggctttatcacctactggatcggctgggtgcgccagatgcccgggaaaggcctggagtggatggggatcatctatcctggtgactctgaaaccagatacagcccgtccttccaaggccaggtcaccatctcagccgacaagtccatcaacaccgcctacctgcagtggagcagcctgaaggcctcggacaccgccatatattactgtgcggggggttcggggatttctacccctatggacgtctggggccaagggaccacggtcaccgtc

SEQ ID NO: 30 - DNA, 1356 bp, Artificial Sequence
  Description of Artificial Sequence Synthetic polynucleotide
gaggtgcagctggttgaatctggcggaggactggtgcagcctggcagatccctgagactgtcttgtgccgccagcggcttcaccttcgacgattatgccatgcactgggtccgacaggcccctggcaaaggacttgaatgggtgtccgccatcacctggaacagcggccacatcgattacgccgatagcgtggaaggccggttcaccatcagcagagacaacgccaagaacagcctgtacctgcagatgaactccctgagagccgaggacaccgccgtgtactactgtgccaaggtgtcctacctgagcaccgccagcagcctggattattggggacagggcacactggtcaccgtgtctagcgcctctacaaagggccctagcgtgttccctctggctcctagcagcaagtctacaagcggaggaacagccgctctgggctgcctggtcaaggattactttcccgagcctgtgaccgtgtcctggaactctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaagatcccgaagtgaagttcaactggtacgtggacggcgtggaagtgcacaatgccaagaccaagcctagagaggaacagtacaacagcacctacagagtggtgtccgtgctgaccgtgctgcatcaggattggctgaacggcaaagagtacaagtgcaaagtgtccaacaaggccctgcctgctcctatcgagaaaaccatctccaaggccaagggccagcctagagaaccccaggtttacacactgcctccaagcagggacgagctgaccaagaatcaggtgtccctgacctgcctcgtgaagggcttctacccttccgatatcgccgtggaatgggagagcaatggccagcctgagaacaactacaagacaacccctcctgtgctggacagcgacggctcattcttcctgtacagcaagctgacagtggacaagtccagatggcagcagggcaacgtgttcagctgcagcgtgatgcacgaggccctgcacaaccactacacccagaagtccctgagcctgtctcctggcaaataa

SEQ ID NO: 31 - DNA, 645 bp, Artificial Sequence
  Description of Artificial Sequence Synthetic polynucleotide
gacatccagatgacacagagccctagcagcctgtctgccagcgtgggagacagagtgaccatcacctgtagagccagccagggcatcagaaactacctggcctggtatcagcagaagcccggcaaggctcctaagctgctgatctacgccgccagcacactgcagtctggcgtgccatctagattttccggcagcggctctggcaccgacttcaccctgaccatatctagcctgcagcctgaggacgtggccacctactactgccagagatacaacagagccccttacaccttcggccagggcaccaaggtggaaatcaagagaacagtggccgctcctagcgtgttcatcttcccaccttccgacgagcagctgaaaagcggcacagcctctgtcgtgtgcctgctgaacaacttctaccccagagaagccaaggtgcagtggaaggtggacaacgctctgcagtccggcaacagccaagagagcgtgacagagcaggacagcaaggactccacctacagcctgagcagcaccctgacactgagcaaggccgactacgagaagcacaaagtgtacgcctgcgaagtgacccaccagggcctttctagccctgtgaccaagagcttcaaccggggcgaatgttaa

SEQ ID NO: 32 - DNA, 333 bp, Artificial Sequence
  Description of Artificial Sequence Synthetic polynucleotide
atggccctgtggatgcgcctcctgcccctgctggcgctgctggccctctggggacctgacccagccgcagcctttgtgaaccaacacctgtgcggctcacacctggtggaagctctctacctagtgtgcggggaacgaggcttcttctacacacccaagacccgccgggaggcagaggacctgcaggtggggcaggtggagctgggcgggggccctggtgcaggcagcctgcagcccttggccctggaggggtccctgcagaagcgtggcattgtggaacaatgctgtaccagcatctgctccctctaccagctggagaactactgcaactag

SEQ ID NO: 33 - protein, 22 aa, Artificial Sequence
  Description of Artificial Sequence Synthetic peptide
STSSEFCSRRYRGPGIHRPVAT

(19) United States
(12) Patent Application Publication
(10) Pub. No.: US 2024/0042009 A1
(43) Pub. Date: Feb. 8, 2024

(54) YEAST-BASED EXPRESSION OF THERAPEUTIC PROTEINS IN VIVO

(71) Applicant: ESPEROVAX INC., Plymouth, MI (US)

(72) Inventor: David James O'Hagan, Whitmore Lake, MI (US)

(21) Appl. No.: 18/038,673

(22) PCT Filed: Nov. 24, 2021
(86) PCT No.: PCT/US21/60865

§ 371 Date: May 24, 2023

Related U.S. Application Data
(60) Provisional application No. 63/118,610, filed on Nov. 25, 2020.

(51) Int. Cl. A61K 39/215; C12N 1/18; C12N 9/42; A61P 31/14; C07K 14/165; A61K 9/00

(52) U.S. Cl. CPC A61K 39/215; C12N 1/18; C12N 9/2442; A61P 31/14; C07K 14/165; A61K 9/0056; A61K 2039/542

(57) ABSTRACT

Various recombinant yeast suitable for use in pharmaceutical compositions expressing therapeutic proteins, food compositions expressing therapeutic proteins, methods of administering to an animal, and related methods, kits, and nucleic acid molecules are described.

### CROSS-REFERENCE TO RELATED APPLICATIONS

The present application claims the benefit of priority to U.S. Provisional Application No. 63/118,610, filed Nov. 25, 2020, the disclosure of which is incorporated herein by reference in its entirety for all purposes.

### FIELD

The disclosure relates to the field of yeast-based delivery vehicles and more particularly, to the oral administration of recombinant yeast cells capable of inducing the expression of therapeutic proteins of interest in vivo. Specific examples relate to recombinant yeast cells, pharmaceutical and food compositions comprising same, methods of producing a therapeutic protein, and methods of treating an animal. The disclosure also relates to various other methods, kits, and nucleic acid molecules.

### BACKGROUND

RNA delivery is an attractive strategy to achieve transient gene expression in gene-based therapies. Despite significant efforts investigating vector-directed RNA transfer, there is still a requirement for better efficiency of delivery in vivo.

Yeast based therapeutic protein platforms have been described for high-volume therapeutic protein production and/or use as an oral vaccination, see, e.g., U.S. Pat. No. 10,117,915. To date, however, such platforms do not efficiently release complex therapeutic proteins that can be induced from host tissue.

### SUMMARY

The present inventors have unexpectedly discovered that regulated permeabilization of the cell wall in recombinant yeast cells engineered to express a therapeutic protein, e.g., an antibody or hormone, can significantly improve protein release by the recombinant yeast. Without wishing to be bound by theory, the present inventors hypothesize that, by inducing regulated permeabilization using one or more methods described herein, the amount of therapeutic protein (e.g., in the form of a protein or containing a packaged nucleic acid sequence encoding the protein) released can be improved as compared to a recombinant yeast cell that is not permeabilized; the degree of recombinant yeast cell viability can be maintained at a higher level as compared to previously described permeabilization methods; the efficacy of the resulting therapeutic protein composition can be increased; and/or protein release can be more selective as compared to previously described methods. One or more of these improvements, in turn, can significantly improve the amount or purity of protein recovered in an in vitro protein production method; and/or the amount of protein (or packaged nucleic acid sequence encoding the protein) released by a recombinant yeast cell in the gastrointestinal tract of a subject having been administered the recombinant yeast cell.

Regulated permeabilization can be induced by inducing expression of a cell wall permeabilizing agent, such as a cell wall degrading enzyme (e.g., mannase, glucanase, chitinase, or combination thereof) or cell wall inhibitor, inducing expression of an inhibitor of cell wall biosynthesis, or by reducing or eliminating expression of a component of the cell wall biosynthesis pathway in a regulated manner. In some embodiments, the cell-wall degrading enzyme is a glucanase, such as a β-glucanase or a β-1,3-glucanase.

In a preferred embodiment, the cells to which the therapeutic protein, or the packaged nucleic acid sequence encoding the protein, is delivered are in vivo, such as, e.g. enterocytes in the gastrointestinal tract of a subject that has been administered the recombinant yeast. In some cases, the yeast are administered orally as a pharmaceutical composition and/or foodstuff. Various exemplary recombinant yeast cells and formulations thereof suitable for oral administration are described herein.

In one aspect the recombinant yeast cell suitable for use in oral administration is derived from a wild-type yeast cell, e.g. Saccharomyces cerevisiae, and comprises a heterologous regulated promoter operably linked to a nucleic acid sequence encoding a cell wall permeabilizing agent, and a heterologous regulated promoter operably linked to a nucleic acid sequence encoding a therapeutic protein. In some embodiments, the nucleic acid encoding the therapeutic protein and the nucleic acid encoding the cell wall permeabilizing agent are under common genetic control. In an exemplary embodiment, the heterologous regulated promoter is the Tet-off regulated promoter.

In some embodiments the recombinant yeast cell further comprises a heterologous regulated promoter operably linked to a nucleic acid sequence encoding at least one viral structural protein or functional fragment thereof, e.g. a capsid protein or functional fragment thereof to form VLPs, and/or a matrix protein or functional fragment thereof to form enveloped VLPs. For example, VLPs as described herein can be formed from one or more structural proteins from SARS-CoV-2, Influenza, Respiratory syncytial virus (RSV), Simian Immunodeficiency Virus (SIV), Human Immunodeficiency Virus (HIV), Noravirus, and the like. In embodiments, nucleic acid sequences encoding VLP-forming protein sequences can include but are not limited to nucleic acid sequences encoding for one or more of a matrix protein, a capsid protein, a GAG protein, a GAG-homology protein, an envelope protein, functional fragments thereof, or combinations thereof. In exemplary embodiments, a VLP-forming protein sequence comprises a GAG protein (e.g., SIV or HIV GAG), or a GAG-homology protein or functional domain thereof, selected from the group consisting of Arc, ASPRV1, a Sushi-Class protein, a SCAN protein, or a PNMA protein. In additional or alternative embodiments, a VLP-forming protein sequence comprises a GAG-homology protein selected from the group consisting of PEG10, RTL3, RTL10, or RTL1. In additional or alternative exemplary embodiments, a VLP-forming protein sequence comprises yeast L-A GAG. In additional or alternative exemplary embodiments, a VLP-forming protein sequence comprises a matrix protein (e.g., Influenza M1 protein). In additional or alternative exemplary embodiments, a VLP-forming protein sequence comprises a capsid protein (e.g., a coronoviral N protein, Influenza NP). In additional or alternative exemplary embodiments, a VLP-forming protein sequence comprises an envelope protein (e.g., coronavirus E).

In some embodiments, the nucleic acid sequence encoding the therapeutic protein further comprises an Internal Ribosome Entry Site (IRES) element inactive in yeast; optionally wherein said IRES element is SEQ ID NO. 6.

In some embodiments the nucleic acid sequence encoding the VLP-forming protein sequence is linked to a nucleic acid binding peptide/protein, and the nucleic acid sequence encoding the therapeutic protein comprises a region encoding at least one nucleic acid binding peptide/protein ligand sequence corresponding to the nucleic acid binding peptide, and a region encoding for the therapeutic protein. In an exemplary embodiment, the nucleic acid binding peptide comprises an MS2 peptide sequence, and the nucleic acid binding peptide ligand sequence comprises an MS2 ligand sequence.

In some embodiments, the therapeutic protein is an antibody or a functional fragment thereof. In some embodiments, the therapeutic protein is an anti-viral antibody. In exemplary embodiments, the therapeutic protein comprises a SARS-CoV-2 spike protein nanobody. In other embodiments, the therapeutic protein is a monoclonal antibody, e.g. an anti-COVID-19 M protein antibody or an anti-COVID-19 NC protein antibody. In some embodiments, the therapeutic protein comprises a C. difficile SLP nanobody. In some embodiments, the therapeutic protein is an anti-inflammatory antibody, e.g., an anti-TNFα monoclonal antibody. In an exemplary embodiment, the therapeutic protein comprises the heavy and light chains from adalimumab (HUMIRA®). In some embodiments, the therapeutic protein is an anti-cancer antibody, e.g., a Herceptin antibody for breast cancer.

In some embodiments, the therapeutic protein is a hormone (e.g. insulin, ghrelin, leptin, and the like), an enzyme (e.g. alcohol dehydrogenase or other detoxifying enzymes), a cytokine (e.g. IL-10), an anti-microbial protein (Iseganan (IB-367) or hLF1-11) a chemokine, a mitogen, an immunogen, (e.g. Covid S protein or fragment thereof), a growth factor (e.g. human growth hormone), or a differentiation factor (OSK, Yamanaka factors for tissue regeneration).

In some embodiments, the recombinant yeast cell comprises a first nucleic acid sequence encoding a regulated promoter, a second nucleic acid sequence encoding a therapeutic protein, and a third nucleic acid sequence encoding a cell wall permeabilizing agent, e.g., a cell wall degrading enzyme. At least one, or each, of the first nucleic acid sequence, second nucleic acid sequence, and third nucleic acid sequences can comprise a nucleic acid sequence that does not occur naturally in the wild-type yeast cell. In some embodiments, expression of the second and/or third nucleic acid sequence is under control of the regulated promoter. In some embodiments, expression of the second and third nucleic acid sequences is under common genetic control of the regulated promoter.

In some embodiments, the recombinant yeast cell comprises a fourth nucleic acid sequence encoding a VLP-forming protein sequence, such as a capsid protein, a matrix protein, a GAG protein, a GAG-homology protein, an envelope protein, functional fragments thereof, or combinations thereof. In some embodiments, the recombinant yeast cell comprises a fourth nucleic acid sequence encoding an enveloped VLP-forming protein sequence, such as a matrix protein, or a functional fragment thereof. In some embodiments, the recombinant yeast cell comprises a fourth nucleic acid sequence encoding at least one VLP-forming protein sequence linked or fused to a reporter polypeptide, e.g., an enzyme or a fluorescent protein, for tracking administration of VLPs to a subject and/or uptake of VLPs by cells of a subject. In some embodiments, the recombinant yeast cell comprises a fourth nucleic acid sequence encoding at least one VLP-forming protein sequence linked to a therapeutic protein, e.g., an enzyme. In some embodiments the VLP-forming protein sequence is linked or fused to a nucleic acid binding peptide, and the nucleic acid sequence encoding the therapeutic protein comprises a region encoding at least one nucleic acid binding peptide ligand sequence corresponding to the nucleic acid binding peptide, and a region encoding for the therapeutic protein. In preferred embodiments, one or more of the foregoing sequences is integrated into the yeast chromosome so as to get stable expression of VLPs.

In one aspect, the present invention provides a plurality of any one of the foregoing recombinant yeast cells, or any one of the recombinant yeast cells described herein, or a combination thereof. The plurality can range from at least 1×10⁶ to about 1×10¹⁵ cells, or from at least 1×10⁷ to about 1×10¹⁴ cells, or from at least 1×10⁸ to about 1×10¹³ cells.

The plurality of recombinant yeast cells can be in a culture medium comprising a density of from about 1×10⁵ cells/mL to about 2×10⁹ cells/mL, preferably from about 1×10⁸ cells/mL to about 2×10⁹ cells/mL. The plurality of recombinant yeast cells can be in a concentrated liquid comprising a density of from about 1×10⁹ cells/mL to about 1×10¹⁰ cells/mL. For example, the liquid can be a concentrated culture medium or the plurality of recombinant yeast cells can be concentrated by separating the cells from a culture medium and resuspending the cells in a buffer. The plurality of cells can be a freeze dried or spray-dried composition. In some cases, the freeze dried composition comprises from at least 1×10⁶ cells/g to 1×10⁹ cells/g. In some cases, the spray-dried composition comprises from at least 1×10⁶ cells/g to 1×10⁹ cells/g. In some cases, yeast cell density can be as high as OD600>200.

Various exemplary pharmaceutical compositions are also described herein.

In one embodiment the pharmaceutical composition comprises an ingestible vessel defining a cavity, such as a capsule, and a recombinant yeast cell disposed in the cavity. The recombinant yeast cell disposed in the cavity can, e.g., be any one of the foregoing recombinant yeast cells, or any one of the recombinant yeast cells described herein, or a combination thereof, e.g., in a liquid, concentrated liquid, or a solid (e.g., freeze dried or spray dried) preparation. In some embodiments, the ingestible vessel comprises from at least 1×10⁶ recombinant yeast cells to about 1×10¹² recombinant yeast cells. In some embodiments, the recombinant yeast cell comprises a regulated promoter operably linked to a nucleic acid sequence encoding a cell wall permeabilizing agent, e.g. a cell wall degrading enzyme and/or a cell wall inhibiting toxin, and a regulated promoter operably linked to a nucleic acid sequence encoding a therapeutic protein. In some embodiments, the recombinant yeast cell further comprises a regulated promoter operably linked to a nucleic acid sequence encoding at least one viral structural protein or functional fragment thereof, e.g. a capsid protein or functional fragment thereof to form VLPs, and/or a matrix protein or functional fragment thereof to form enveloped VLPs.

In some embodiments the VLP-forming protein sequence is fused to a nucleic acid binding peptide, and the nucleic acid sequence encoding the therapeutic protein comprises a region encoding at least one nucleic acid binding peptide ligand sequence corresponding to the nucleic acid binding peptide, and a region encoding for the therapeutic protein. In an exemplary embodiment, the nucleic acid binding peptide comprises an MS2 peptide sequence, and the nucleic acid binding peptide ligand sequence comprises an MS2 ligand sequence.

Another exemplary pharmaceutical composition comprises a plurality of recombinant yeast cells as described herein spray-dried in combination with alginate or chitosan, or a combination thereof and one or more excipients. Suitable excipients include, but are not limited to MgCl₂, CaCl₂), and combinations thereof. See, Szekalska et al., Materials (Basel). 2018, September 11 (9):1522; and U.S. Pat. No. 9,700,519. In some embodiments, the recombinant yeast cell comprises a regulated promoter operably linked to a nucleic acid sequence encoding a cell wall permeabilizing agent, e.g. a cell wall degrading enzyme and/or a cell wall inhibiting toxin, and a regulated promoter operably linked to a nucleic acid sequence encoding a therapeutic protein. In some embodiments, the recombinant yeast cell further comprises a regulated promoter operably linked to a nucleic acid sequence encoding at least one viral structural protein or functional fragment thereof, e.g. a capsid protein or functional fragment thereof to form VLPs, and/or a matrix protein or functional fragment thereof to form enveloped VLPs.

In some embodiments the VLP-forming protein sequence is fused to a nucleic acid binding peptide, and the nucleic acid sequence encoding the therapeutic protein comprises a region encoding at least one nucleic acid binding peptide ligand sequence corresponding to the nucleic acid binding peptide, and a region encoding for the therapeutic protein. In an exemplary embodiment, the nucleic acid binding peptide comprises an MS2 peptide sequence, and the nucleic acid binding peptide ligand sequence comprises an MS2 ligand sequence.

Various food compositions are also described herein.

In one embodiment the food composition comprises at least one foodstuff and at least one pharmaceutical composition comprising an ingestible vessel defining a cavity and a recombinant yeast cell described herein or composition comprising a plurality of recombinant yeast cells described herein disposed in the cavity. In another embodiment the food composition comprises at least one foodstuff, and a plurality of pharmaceutical compositions, each of which comprises a polymeric shell defining a cavity and a plurality of recombinant yeast cells described herein disposed in the cavity. In another embodiment the food composition comprises at least one foodstuff and a pharmaceutical composition comprising a plurality of recombinant yeast cells described herein disposed in a cavity defined by a polymeric shell. In another example, a food composition comprises a matrix comprising at least one foodstuff and a pharmaceutical composition comprising a plurality of recombinant yeast cells described herein. In some cases, the pharmaceutical composition is admixed with the foodstuff matrix.

In some embodiments, the nucleic acid sequence encoding the cell wall permeabilizing agent comprises SEQ ID NO. 1, which encodes for an exemplary secreted beta-glucanase cell wall degrading enzyme useful in the methods, compositions, and kits of the present invention. In some embodiments, the nucleic acid sequence encoding the cell wall permeabilizing agent comprises SEQ ID NO. 2, which encodes for an exemplary secreted chitinase cell wall degrading enzyme useful in the methods, compositions, and kits of the present invention.

In some embodiments, the cell wall permeabilizing agent is a cell wall inhibiting toxin, and the nucleic acid sequence comprises at least 25, 50, 100, 125, or 150 contiguous amino acids of, or all of, SEQ ID NO. 3. In some cases, the toxin comprises no more than 1, 2, 3, 4, or 5 single amino acid substitutions, deletions, and/or additions relative to the protein encoded by SEQ ID NO. 4 or the polypeptide sequence set forth in SEQ ID NO. 3. In some cases, the toxin is at least 80%, 85%, 90%, 95%, or at least 99%, identical to a secreted protein sequence encoded by SEQ ID NO. 4. In some cases, the nucleic acid encodes the cell wall inhibiting toxin encoded by SEQ ID NO. 4. In some cases, the exemplary cell wall inhibiting toxin comprises SEQ ID NO. 3.

Additional understanding of the invention, including the exemplary recombinant yeast cells suitable for oral administration, pharmaceutical compositions, food compositions, methods of producing same, methods of administering to an animal, and related methods, kits, and nucleic acid molecules, can be obtained by reviewing the detailed description of selected examples, below, and the appended drawings.

### BRIEF DESCRIPTION OF THE DRAWINGS

FIG. 1 illustrates a schematic diagram of an expression cassette for incorporating a gene of interest, e.g., yGFP into a VLP by including an MS2 binding site array. RNA transcripts containing the gene of interest and MS2 binding site array can be packaged into VLPs that are at least partially formed from a fusion protein containing an MS2 binding protein sequence.

FIG. 2 illustrates an exemplary construct encoding an HIV GAG-GFP fusion under the control of an ADH2 promoter and a beta-glucanase (Egress 1) under the control of an ADH2 promoter.

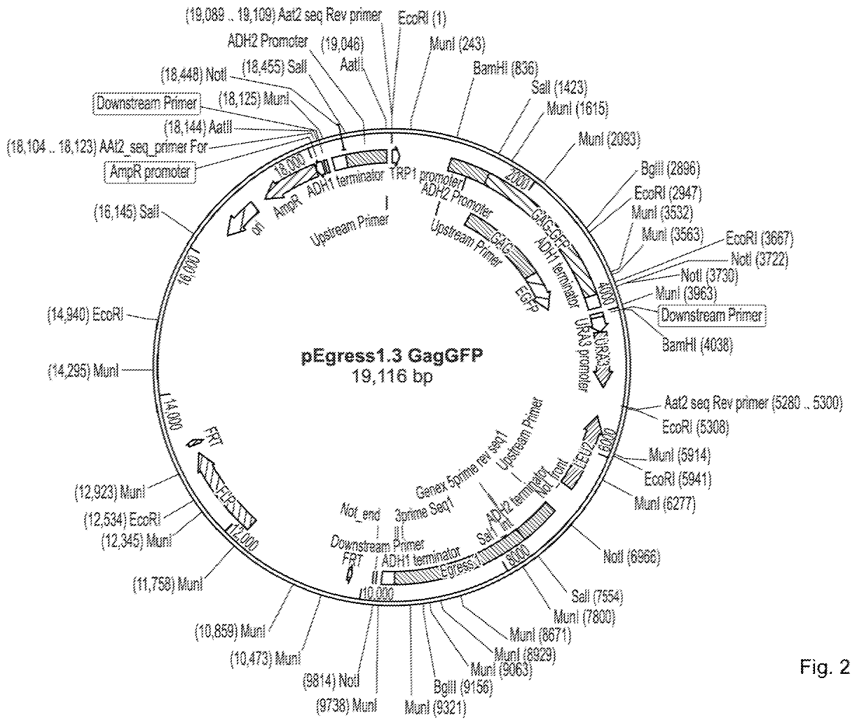

FIG. 3 illustrates an exemplary construct encoding an HIV GAG-MS2 fusion protein, a beta-glucanase, and a nucleic acid sequence encoding an mRNA that encodes EGFP and includes multiple MS2-protein binding sites.

FIG. 4 illustrates a schematic of eVLPs that carry an mRNA payload for expression of a therapeutic protein in target cells of an administered host that take up (e.g., phagocytose) the eVLPs.

FIG. 5 illustrates RT-PCR results of eVLPs using EGFP-specific primers demonstrating presence of packaged EGFP mRNA in the eVLPs.

FIG. 6 illustrates a fluorescence microscope image of recombinant yeast cells secreting eVLPs carrying an EGFP mRNA payload.

FIG. 7 illustrates dendritic cells (left) expressing mRNA encoded EGFP from phagocytosed eVLPs (right).

FIG. 8 illustrates transmission electron micrographs of eVLPs obtained from culture media.

FIG. 9 illustrates the linearized pOCH1 construct.

FIG. 10 illustrates the linearized viability switch construct utilizing the pol3 gene.

FIG. 11 provides PCR data to confirm that the pOCH1 construct had integrated into the yeast genome. Briefly, genomic DNA was isolated from three transformants, and that DNA was used as template for PCR. Using the Cre and Amp primers indicated that all three transformants tested integrated the construct into the yeast genome.

FIG. 12 illustrates the protocol followed for demonstrating inhibition of target protein expression in yeast cells via insertion of an IRES element.

FIG. 13 illustrates the reduction in protein expression in recombinant yeast comprising an IRES element upstream of the coding sequence for the target gene.

FIG. 14 shows that gene-specific RT-PCR products were obtained from the VLPs containing mRNA of the gene of interest using gene specific primers.

FIG. 15 is a schematic representation of an exemplary SARS-CoV-2 spike protein nanobody recombinant DNA construct.

FIG. 16 is a schematic representation of an exemplary SARS-Co-V-2 spike protein monoclonal antibody recombinant DNA construct.

FIG. 17 is a schematic representation of an exemplary adalimumab (HUMIRA©) antibody recombinant DNA construct.

FIG. 18 is a schematic representation of an exemplary insulin recombinant DNA construct.

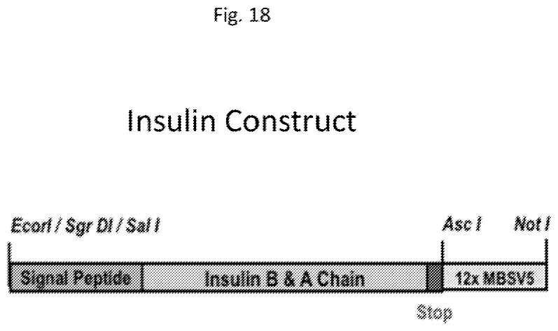

FIG. 19 is a graph illustrating serum antibody levels in mice that orally received recombinant yeast cells engineered to express a GAG-GFP fusion and a cell-wall permeabilizing agent.

FIG. 20 is a graph illustrating serum antibody levels in mice that orally received recombinant yeast cells engineered to express a GAG-MS2 fusion protein and a cell-wall permeabilizing agent, and to produce RNA transcripts encoding SARS-CoV-2 spike protein and a MS2 binding site.

### DETAILED DESCRIPTION

The following detailed description and the appended drawings describe and illustrate various examples of recombinant yeast suitable for use in producing a therapeutic protein. Such recombinant yeast cells can be used for making therapeutic protein in vivo, and in related methods, kits, and nucleic acid molecules. The description and drawings are provided to enable one skilled in the art to make and use one or more recombinant yeast suitable for use in oral administration, pharmaceutical compositions, kits, and nucleic acid molecules and to perform the exemplary methods. They are not intended to limit the scope of the claims in any manner.

As used herein, the term “animal” refers to a vertebrate. The term includes mammals, birds, fish, reptiles, and amphibians. As such, the term includes humans, domesticated pets, such as dogs and cats, feral cats, horses, cattle, and other vertebrate animals. The term also includes agriculturally important animals such as domesticated pigs, chickens, cows, sheep, goats, horses, donkeys, mules, ducks, geese and turkeys.

As used herein, the term “cavity” refers to an open space defined by an object. On its own, the term does not require any specific structure or physical properties and includes, for example, spaces with exposed openings and enclosed spaces.

As used herein, the term “common genetic control” a property of multiple nucleic acid sequences being regulated by the same promoter. The term includes nucleic acid arrangements in which the multiple nucleic acid sequences are positioned downstream of a single promoter that regulates the expression of both nucleic acid sequences. The term also includes nucleic acid arrangements in which one of the multiple nucleic acid sequences is positioned downstream of a first copy of the promoter and another of the multiple nucleic acid sequences is positioned downstream of a second copy of the promoter. It should be appreciated, that where multiple copies of a promoter are used in a scheme for expression of proteins under common genetic control, the copies need not be identical in sequence and minor variations in promoter sequence are tolerated so long as functional equivalency is maintained.

As used herein, the term “ingestible” refers to the ability of a referenced element to be ingested by an animal.

As used herein, the term “regulated promoter” refers to a region of DNA that initiates transcription of a particular gene under specific conditions. The term includes inducible promoters and repressible promoters. Examples of inducible promoters include both positive inducible promoters, i.e., inducible promoters that are activated in the presence of the inducer, such as by interaction between the inducer and an activator molecule to enable binding of the combined entity to the inducible promoter to effect transcription of downstream genes controlled by the inducible promoter, and negative inducible promoters, i.e., inducible promoters that are activated in the presence of the inducer, such as by interaction between the inducer and a repressor to block or disable binding of the repressor to the inducible promoter, thereby removing suppression of transcription of downstream genes controlled by the inducible promoter. Examples of repressible promoters include both positive repressible promoters, i.e., promoters that are repressed in the presence of the repressor, such as by interaction between the repressor and an activator molecule to block or disable binding of the activator molecule to the repressible promoter, thereby removing activation of transcription of downstream genes controlled by the repressible promoter, and negative repressible promoters, i.e., promoters that are repressed in the presence of the repressor, such as by interaction between the repressor and a corepressor molecule to enable binding of the combined entity to the repressible promoter to effect transcription of downstream genes controlled by the repressible promoter. The term also includes promoters that can be regulated as both a positive inducible promoter and a negative inducible promoter, and promoters that respond to environmental queues, such as the presence or absence of light, the absence of a particular molecule, and any other promoter that can be specifically regulated by providing or removing a particular molecule or environmental queue.

As used herein, the term “vessel” refers to a structure capable of partially or completely containing a substance, such as one or more recombinant yeast cells. On its own, the term does not require any specific structure or physical properties and includes, for example, open structures, closed structures, single component structures, multi-component structures, rigid structures, and flexible structures.

As used herein, the term “single-domain antibody” (sdAb), also known as a nanobody, is an antibody fragment consisting of a single monomeric variable antibody domain. Like a whole antibody, it is able to bind selectively to a specific antigen. With a molecular weight of only 12-15 kDa, single-domain antibodies are much smaller than common antibodies (150-160 kDa) which are composed of two heavy protein chains and two light chains. As one example, camelids have been found to produce heavy-chain-only antibodies (HCAbs), which contain a single variable domain (VHH) instead of two variable domains (VH and VL) that make up the equivalent antigen-binding fragment (Fab) of conventional immunoglobulin G (IgG) antibodies (Wrapp et al., (2020). Structural Basis for Potent Neutralization of Betacoronaviruses by Single-Domain Camelid Antibodies. Cell 181: 1004-1015). This single variable domain, in the absence of an effector domain, is a single-domain antibody, VHH, and typically can acquire affinities and specificities for antigens comparable to conventional antibodies.

As used herein, the term “virus like particle” or “VLP” refers to a non-infectious nanostructure composed of viral structural proteins and lacking viral nucleic acid. A virus like particle morphologically resembles a virus, but, without more, lacks the ability to infect a host cell. VLPs are typically comprised of at least one viral structural component that forms at least a part of a VLP shell, such as a capsid protein, a matrix protein, a GAG protein, a GAG-homology protein, an envelope protein, functional fragments thereof, or combinations thereof.

As used herein, the term “enveloped virus like particle” refers to a VLP that includes a host-cell derived membrane. The acronym eVLP refers to the term “enveloped virus like particle.” neVLPs can comprise at least one matrix protein. In some cases, an eVLP may comprise 2 or 3, or more, different matrix proteins.

As used herein, the term “non-enveloped virus like particle” refers to a VLP that does not include a host-cell derived membrane. The acronym neVLP refers to the term “non-enveloped virus like particle.” neVLPs can comprise at least one capsid protein. In some cases, an neVLP may comprise 2 or 3, or more, different capsid proteins.

VLPs, including neVLPs and/or eVLPs can be engineered to include a nucleic acid binding peptide, which in turn can bind a specific nucleic acid binding site sequence. As described further below, one exemplary nucleic acid binding peptide is found in the MS2 coat protein, which binds an, e.g., 19-nucleotide, ribosomal binding site of the MS2 replicase mRNA, which folds into a hairpin loop structure. Typically one or more nucleic acid binding sites are included as a repeated array of nucleic acid binding sites to increase the amount of cognate protein localized to the nucleic acid. In some cases, the repeated sequence can compromise genetic stability of the recombinant coding sequence. In one embodiment, the nucleic acid binding sites in the repeated array are synonymous binding sites that are different in sequence and yet retain the cognate protein binding function. Such arrays of synonymous nucleic acid binding sites are described in, e.g., Wu et al., Genes Dev. 2015 Apr. 15 (29(8); 876-886, as well as WO 2020/237100, the contents of which are incorporated herein in their entirety for all purposes.

Such VLPs engineered to include a nucleic acid binding peptide can be used to deliver a nucleic acid encoding a therapeutic protein to an endogenous host cell, e.g. enterocyte, so as to have the endogenous host cell produce and express the therapeutic protein directly. In one embodiment, the VLP-forming protein sequence comprises at least one capsid protein, matrix protein, GAG protein, GAG-homology protein, or envelope protein fused to the nucleic acid binding element. For example, the VLP-forming sequence can comprise a GAG-MS2 fusion, such as the GAG-MS2 fusion set forth in SEQ ID NO. 5. For example, a GAG-MS2 fusion protein can comprise at least 25, 50, 100, 125, or 150 contiguous amino acids of, or of, SEQ ID NO. 5, and/or can be at least 80%, 85%, 90%, 95%, or 99% identical to at least 25, 50, 100, 125, or 150 contiguous amino acids of, or of, SEQ ID NO. 5, and/or can comprise no more than 1, 2, 4, or 5, single amino acid insertions, substitutions, and/or deletions of SEQ ID NO. 5. For example, a GAG-MS2 fusion protein can comprise no more than 1, 2, 4, or 5, single amino acid insertions, substitutions, and/or deletions of a contiguous amino acid region of SEQ ID NO. 5 of at least 25, 50, 100, 125, or 150 amino acids in length.

Alternative fusions for binding a nucleic acid include, but are not limited to a fusion of nanoparticle producing proteins like capsid proteins, matrix proteins, GAG proteins, GAG-homology proteins, envelope proteins, or Influenza M1 to a nucleic acid binding peptide like Bacteriophage capsids MS2, PP7, or other RNA virus mRNA binding capsids. Others include matrix proteins like nucleic acid binding peptide (NC), and a fusion of HBV nucleocapsid protein to a nucleic acid binding peptide.

Additionally, or alternatively, such VLPs can be used to deliver a nucleic acid encoding a reporter to increase a reporter signal by expressing the reporter in a cell that takes up the VLP.

Alternative nucleic acid binding peptides and corresponding nucleic acid binding site sequences, include but are not limited to those described in U.S. 2017/0233762, the contents of which are herein incorporated by reference in the entirety for all purposes including but not limited to RNA ligand sequences and RNA binding peptide sequences and their use. A skilled person will appreciate that multiple RNA binding peptide sequences (e.g., in a VLP fusion protein) and their ligands (e.g., in the target nucleic acid to be packaged) can be incorporated to package multiple copies of the same nucleic acid or to package multiple different nucleic acids.

Where polypeptide sequences are disclosed herein, e.g., by sequence listing, it is understood that such polypeptides can include an N-terminal secretion signal suitable to support secretion of a mature-form (e.g., wherein the signal sequence is cleaved) polypeptide from a host organism such as a yeast cell. Where a signal peptide is already present in the disclosed sequence, a skilled person will appreciate that such a sequence also discloses the mature form of the polypeptide after cleavage of the signal peptide. Moreover, a skilled person will appreciate that a signal sequence can be replaced with a signal sequence optimized for a host organism described herein.

Described herein are methods and compositions that provide increased therapeutic protein release by regulated permeabilization of a recombinant yeast cell that produces said therapeutic protein. As described herein in various embodiments, this improved protein release can be provided by regulated induction of expression of a cell wall degrading enzyme, regulated repression of expression of a component of a cell wall biosynthesis pathway, or regulated induction of expression of an inhibitor of cell wall biosynthesis.

A recombinant yeast cell according to the present invention comprises a first nucleic acid sequence encoding a regulated promoter; a second nucleic acid sequence encoding a therapeutic protein; and a third nucleic acid sequence encoding a cell wall permeabilizing agent. In some embodiments, at least one, or each, of the first nucleic acid sequence, second nucleic acid sequence, and third nucleic acid sequence comprises a nucleic acid sequence that does not occur naturally in the wild-type yeast cell and that has been artificially introduced into the wild-type yeast cell to produce recombinant yeast cell. In some cases, expression of the second nucleic acid sequence and/or the third nucleic acid sequence are under common genetic control of the regulated promoter. Accordingly, the recombinant yeast cell has been genetically modified to include at least one therapeutic protein gene, at least one cell wall permeabilizing agent gene, and at least one regulated promoter. The first, second, and/or third nucleic acid sequences can be present on one or more plasmids. In some cases, at least one, or all of the first, second, and third nucleic acid sequences are inserted into the genome of the yeast cell at the same, or at a different, locus.

In some embodiments, the recombinant yeast cell comprises a fourth nucleic acid sequence encoding a VLP-forming protein sequence, such as a capsid protein, a matrix protein, a GAG protein, a GAG-homology protein, an envelope protein, functional fragments thereof, or combinations thereof. In some embodiments, the recombinant yeast cell comprises a fourth nucleic acid sequence encoding an enveloped VLP-forming protein sequence, such as a matrix protein, or a functional fragment thereof. In some embodiments, the recombinant yeast cell comprises a fourth nucleic acid sequence encoding at least one VLP-forming protein sequence fused to a reporter polypeptide, e.g., an enzyme or a fluorescent protein, for tracking administration of VLPs to a subject and/or uptake of VLPs by cells of a subject.

The recombinant yeast cell can be produced from any suitable wild-type yeast cell and a skilled artisan will be able to select a wild-type yeast cell for producing a recombinant yeast cell according to a particular embodiment based on various considerations, including the nature of the therapeutic protein to be delivered or induced, the cell wall permeabilizing agent to be used in the particular embodiment, the availability of the wild-type yeast cell, the relative ease with which the wild-type yeast cell can be transformed with a vector or vectors comprising the first, second, and third nucleic acid sequences, the relative ease with which the wild-type yeast cell can be grown in production level quantities, and the length of time over which the wild type yeast cell remains stable after being freeze-dried or processed using other techniques to achieve suspension of growth and other activities. Examples of suitable wild type yeast cells include Saccharomyces cerevisiae (S. cerevisiae, also known as “baker's yeast”), Pichia pastoris, and Hansenula polymorpha.

The inventors have determined that S. cerevisiae is useful as a wild-type yeast cell in production of a recombinant yeast cell according to embodiments of the invention at least because of its ready availability, well-characterized transformation effectiveness, and well-characterized handling techniques. The inventors have identified S. cerevisiae strain Sc1602 MAT alpha, ura3−, leu−, pep4−, och1− as a useful wild-type yeast cell in production of a recombinant yeast cell according to embodiments of the invention.

The first nucleic acid sequence encodes a regulated promoter. The regulated promoter can comprise any suitable regulated promoter and a skilled artisan will be able to select a regulated promoter for a recombinant yeast cell according to a particular embodiment based on various considerations, including the nature of the wild-type yeast cell used in the production of the recombinant yeast cell, any desired type of control over the production of the therapeutic protein and/or cell wall permeabilizing agent, and any equipment and/or supplies needed to control expression of the therapeutic protein and cell wall permeabilizing agent using a particular inducible promoter. Examples of suitable regulated promoters include inducible promoters, including positive inducible promoters, negative inducible promoters, and inducible promoters that can be regulated as both a positive inducible promoter and a negative inducible promoter, and repressible promoters, including positive repressible promoters, negative repressible promoters, and repressible promoters that can be regulated as both a positive repressible promoter and a negative repressible promoter. Examples of suitable regulated promoters include the Gall inducible promoter, which activates transcription of genes controlled by the promoter in the presence of galactose, and the ADH2 promoter, which activates transcription in the absence of glucose. Other examples of regulated promoters considered suitable include, but are not limited to, PTet, pTP1, pTEF1, pPYK1, pADH1, FMD1, pHXT7, pGAL1, pGAL7, pGAL10, pPHO5, pCUP1, and pDAN1.

The inventors have determined that the Tet-off regulated promoter, a positive repressible promoter, is particularly advantageous for inclusion as the regulated promoter in recombinant yeast cells according to the invention. In the Tet-off system, transcription of genes controlled by the regulated promoter is turned off when tetracycline or one of its derivatives is present. The inventors consider the inclusion of this regulated promoter particularly advantageous at least because of the production methods it enables. For example, as described in detail below, inclusion of this regulated promoter in a recombinant yeast cell enables a method in which a culture of recombinant yeast cells is grown in a laboratory environment in the presence of tetracycline or a tetracycline derivative. During this stage of the method, the genes controlled by the Tet-off system in the recombinant yeast cells in the culture, such as the nucleic acid sequence encoding the therapeutic protein and/or the nucleic acid sequence inducing cell wall permeabilization, such as a cell wall degrading enzyme, are not transcribed. The tetracycline or tetracycline derivative can be removed at a later time. For example, a sufficient amount of repressor can be removed for a predefined period of time after a culture of the cells has achieved a sufficient density or growth phase in the culture, thereby activating transcription of the nucleic acid sequence that encodes the therapeutic protein and/or the nucleic acid sequence that encodes a cell wall degrading enzyme for the length of the predefined period of time. This enables production of a desired amount of protein and/or cell wall degrading enzyme prior to harvesting the recombinant yeast cells in the culture. In turn, this ensures that, when the recombinant yeast cells are ingested by a patient to be treated, such as when the patient ingests freeze-dried recombinant yeast, an amount of therapeutic protein and/or cell wall degrading enzyme are available immediately, which can positively impact the efficacy of the therapeutic protein.

As another example, a positive repressible promoter can be used to regulate cell wall permeability by regulated repression of a cell wall biosynthesis pathway. For example, a recombinant yeast cell can be engineered to include a positive repressible promoter operably linked to a component of a cell wall biosynthesis pathway and to express a therapeutic protein, e.g., in a regulated fashion. The recombinant yeast cell can be cultured under conditions to permit cell wall biosynthesis and then subsequently cell wall biosynthesis can be repressed by removal of the repressor. In some embodiments, the regulated repression of a cell wall biosynthesis pathway is provided by promoter replacement or insertion of a positive repressible promoter operably linked to an endogenous component of a cell wall biosynthesis pathway. Alternatively, an endogenous cell wall biosynthesis pathway component can be knocked out and an alternate, e.g., copy, introduced into the recombinant yeast cell that is operably linked to a positive repressible promoter.

As described herein, in some embodiments, the therapeutic protein and cell wall permeabilizing agent (e.g., cell wall degrading enzyme, cell wall biosynthesis toxin, etc.) are under the common genetic control of a regulatable promoter. Alternatively, in some embodiments, the therapeutic protein and cell wall permeabilizing agent are differentially regulated. In some embodiments, the regulated promoter is operably linked to the nucleic acid sequence encoding the cell wall permeabilizing agent. In some embodiments, a different, e.g., regulated, promoter is operably linked to the nucleic acid sequence encoding the therapeutic protein.

In some cases, the promoter operably linked to the cell-wall permeabilizing agent is selected to induce or de-repress expression of the cell wall permeabilizing agent after the recombinant yeast have been cultured to a sufficient density (e.g., 1×10⁸ cells/mL, OD₆₀₀ between 100-300) or growth phase (e.g., log phase, mid-log phase, or late-log phase growth). In some cases, the promoter operably linked to the cell-wall permeabilizing agent is selected to induce or de-repress expression of the cell wall permeabilizing agent after the recombinant yeast have been harvested or after the recombinant yeast have been administered to a subject.

In some cases, the promoter operably linked to the protein or component thereof is selected to induce or de-repress expression of the therapeutic protein prior to administration of the recombinant yeast to a subject. For example, therapeutic protein production can be de-repressed or induced during culture of the recombinant yeast cells. In some methods of the present invention, therapeutic protein expression is induced or de-repressed and then expression of the permeabilizing agent is induced or de-repressed. In some cases, the yield of expressed protein can be enhanced by inducing expression of cell wall permeabilizing agent after induction of protein expression. In other cases, e.g., where inefficient release of protein overwhelms the secretory capacity of the host cell, it may be preferable to induce expression of the cell wall permeabilizing agent prior to, or at the same time, as inducing the expression of the protein. As described herein, one exemplary method for simultaneous induction of both therapeutic protein and cell wall permeabilization agent is to operably link the nucleic acid sequences encoding both the therapeutic protein and the permeabilization agent to a regulatable common genetic control element.

In some embodiments, the nucleic acid sequence encoding the cell-wall permeabilizing agent is under control of a regulated promoter and the nucleic acid sequence encoding the therapeutic protein is constitutively expressed.

The second nucleic acid sequence encodes a therapeutic protein, i.e. a protein capable of exerting a therapeutic effect, such as an antibody, a hormone, an enzyme, a cytokine, a chemokine, a mitogen, an immunogen, a growth factor, a differentiation factor, and the like. In some embodiments, the therapeutic protein is an antibody or a functional fragment thereof. In some embodiments, the therapeutic protein is an anti-viral antibody. In exemplary embodiments, the therapeutic protein comprises a SARS-CoV-2 spike protein nanobody, or a SARS-CoV-2 spike protein monoclonal antibody. In some embodiments, the therapeutic protein is an anti-inflammatory antibody, e.g., an anti-TNFα monoclonal antibody. In an exemplary embodiment, the therapeutic protein comprises the heavy and light chains from adalimumab (HUMIRA®). In some embodiments, the therapeutic protein is an anti-cancer antibody. In some embodiments, the therapeutic protein may be used to replace or enhance a defective endogenous protein or to compensate for lack of a particular gene product, by encoding a therapeutic product or an enzyme needed to produce a functional protein. In some embodiments, the therapeutic protein may be used as a vaccine to provoke an immune response, by encoding an immunogen than can be produced and/or presented more accurately and/or more efficiently in vivo by a human cell.

Also, while the illustrated embodiment shows a single nucleic acid sequence encoding a single therapeutic protein, it is noted that multiple nucleic acid sequences that each encode a distinct therapeutic protein, or a component thereof, can be included in a recombinant yeast cell according to an embodiment. Examples of suitable numbers of nucleic acid sequences that each encode an protein include, but are not limited to, one, at least one, more than one, two, a plurality, three, four, five, six, seven, eight, nine, ten, and more than ten. Also, the second nucleic acid sequence can encode a naked VLP protein or an enveloped VLP protein.

In some cases, methods for producing therapeutic proteins in a permeabilized yeast further include inhibiting cell replication during the induction phase, as described in co-pending U.S. Provisional Patent Application No. 63/118,611, the contents of which is incorporated by reference herein in its entirety. In some embodiments, the inhibition of replication can improve therapeutic protein production by reducing the metabolic burden of replication. Cell replication can be inherently inhibited by inhibiting cell wall production (e.g., using a Killer Toxin) inhibiting cell wall maintenance (e.g. using a cell wall degrading enzyme), or inhibiting genome replication, inducing expression of a checkpoint activator, such as TEL1 or Mps1.

In some cases, genome replication is inhibited by inhibiting expression or activity of endogenous DNA polymerase. In some cases, DNA polymerase is inhibited by removing all or part of the genomic region encoding the endogenous yeast DNA polymerase. In some cases, methods of producing VLPs described herein include inducing expression of a recombinant recombinase, such as CR1 recombinase, and thereby inducing recombination at one or more, preferably two lox sites (e.g., loxP) in the genome at the genomic region encoding the endogenous DNA polymerase Typically, the lox sites are positioned to flank an essential region of the endogenous DNA polymerase. In some embodiments, the CRE recombinase is under the genetic control of a regulatable promoter that is common to a nucleic acid sequence encoding a cell-wall permeabilizing agent and/or a nucleic acid sequence encoding a therapeutic protein, and/or a nucleic acid sequence encoding a VLP-forming protein sequence, such as a GAG protein (e.g., SIV or HIV GAG), GAG homology protein (e.g., PEG10), a matrix protein (e.g., influenza M), a capsid protein (e.g., coronavirus N or influenza NP), an envelope protein (e.g. coronavirus E), or a combination thereof. Thus, in some embodiments, yeast host cells described herein contain one or more recombination sites, such as loxP sites at or flanking a DNA polymerase encoding genomic region, and a nucleic acid encoding a heterologous recombinase, such as a CRE recombinase. In some embodiments, the lox sites can additionally flank a cytotoxic agent operably linked to a regulatable promoter. In this way, the cytotoxic agent is produced exclusively in replication competent cells.

A recombination based approach for inhibiting cell replication can be particularly advantageous in forming a pharmaceutical composition suitable for administration to a mammalian subject, wherein the pharmaceutical composition contains, or is likely to contain, at least a portion of whole yeast cells because such cells will not replicate. For example, in some embodiments, VLPs described herein are induced with simultaneous or sequential recombination to inhibit replication, e.g., with simultaneous or sequential permeabilization, cell culture supernatant containing VLPs are collected and used to form the pharmaceutical composition.

In some embodiments, inhibiting expression of the therapeutic gene in yeast cells may be desirable, in order to preferentially direct expression of the encoded protein to the endogenous cellular machinery of the mammalian subject so as to ensure proper folding and/or appropriate post-translational modification, e.g., glycosylation, of the encoded protein. Briefly, there are a number of Internal Ribosomal Entry Site (IRES) elements located in the RNA genomes of e.g., encephalomyocarditis virus, poliovirus, and hepatitis C virus that do not function in living S. Cerevisiae, and that can be advantageously incorporated into the present invention to inhibit expression of the therapeutic protein by the yeast, see, e.g., Eystafieva A G et al., (1993) FEBS Lett 335: 273-276; Coward P and Dasgupta A (1992) J Virol 66: 286-295; Das S et al., (1998) Front Biosci 3: D1241-D1252; Thompson S R et al., (2001) PNAS 98(23): 12972-12977.

In an exemplary embodiment, an IRES element from the Encephalomyocarditis Virus is placed 5′ of the therapeutic protein gene, for example in the 5′ UTR of the therapeutic protein gene, see, e.g., B Walch, T Breinig, MJ Schmitt, and F Breinig, Gene Therapy (2012) 19, 237-245. In an exemplary embodiment, the IRES element is regulated by the Gal promoter and is switched on in the late phases of yeast production, such that the mRNA is produced but no corresponding protein is made. The mRNA can then be captured by the GAG-MS2 fusion protein and incorporated into the VLPs for secretion past the destabilized cell wall. The particles can then be taken up by endogenous cells in the mammalian subject, e.g., enterocytes or other intestinal epithelial cells, dendritic cells or other immune cells, and the like, where the mRNA is unpackaged and translated and the protein expressed. These embodiments are particularly advantageous for therapeutic protein expression requiring precise folding and/or post-translational modification, e.g. for antibody or protein replacement therapies, or for immunogen expression when inaccurate translation can trigger aberrant immune reactivity and/or autoimmunity.

In an exemplary embodiment, the IRES element comprises

(SEQ ID NO. 6)
CCCCTCTCCCTCCCCCCCCCCTAACGTTACTGGCCGAAGCCGCTTGGAAT
AAGGCCGGTGTGCGTTTGTCTATATGTTATTTTCCACCATATTGCCGTCT
TTTGGCAATGTGAGGGCCCGGAAACCTGGCCCTGTCTTCTTGACGAGCAT
TCCTAGGGGTCTTTCCCCTCTCGCCAAAGGAATGCAAGGTCTGTTGAATG
TCGTGAAGGAAGCAGTTCCTCTGGAAGCTTCTTGAAGACAAACAACGTCT
GTAGCGACCCTTTGCAGGCAGCGGAACCCCCCACCTGGCGACAGGTGCCT
CTGCGGCCAAAAGCCACGTGTATAAGATACACCTGCAAAGGCGGCACAAC
CCCAGTGCCACGTTGTGAGTTGGATAGTTGTGGAAAGAGTCAAATGGCTC
TCCTCAAGCGTATTCAACAAGGGGCTGAAGGATGCCCAGAAGGTACCCCA
TTGTATGGGATCTGATCTGGGGCCTCGGTGCACATGCTTTACATGTGTTT
AGTCGAGGTTAAAAAAACGTCTAGGCCCCCCGAACCACGGGGACGTGGTT
TTCCTTTGAAAAACACGATGATAATATGGCCACAACC.

The third nucleic acid sequence can encode a cell wall permeabilizing agent such as a cell wall degrading enzyme. The cell wall degrading enzyme can comprise any suitable cell wall degrading enzyme and a skilled artisan will be able to select a cell wall degrading enzyme for a recombinant yeast cell according to a particular embodiment based on various considerations, including the nature and size of the therapeutic protein encoded by the second nucleic acid sequence, the number of different nucleic acids that encode proteins included in the recombinant yeast cell, the nature of the cell wall of the recombinant yeast cell, and other considerations. Examples of suitable cell wall degrading enzymes include, but are not limited to, a glucanase enzyme such as a β-1,3-glucanase, a mannanase enzyme, a chitinase, and other enzymes capable of degrading a yeast cell wall.

In some cases, the β-1,3-glucanase comprises at least 25, 50, 100, 125, or 150 contiguous amino acids of, or all of, SEQ ID NO. 7. In some cases, the β-1,3-glucanase comprises no more than 1, 2, 3, 4, or 5 single amino acid substitutions, deletions, and/or additions relative to the protein encoded by SEQ ID NO. 1 or the polypeptide sequence set forth in SEQ ID NO. 7. In some cases, the β-1-3-glucanase is at least 80%, 85%, 90%, 95%, or at least 99%, identical to a secreted protein sequence encoded by SEQ ID NO. 1. In some cases, the β-1-3-glucanase is at least 80%, 85%, 90%, 95%, or at least 99% identical to at least 25, 50, 100, 125, or 150 contiguous amino acids of, or all of, SEQ ID NO. 7. In some cases, the glucanase comprises no more than 1, 2, 4, or 5, single amino acid insertions, substitutions, and/or deletions of a contiguous amino acid region of SEQ ID NO. 7 of at least 25, 50, 100, 125, or 150 amino acids in length.

In some cases, the mannanase comprises at least 25, 50, 100, 125, or 150 contiguous amino acids of, or all of, SEQ ID NO. 8. In some cases, the mannanase comprises no more than 1, 2, 3, 4, or 5 single amino acid substitutions, deletions, and/or additions relative to the polypeptide sequence set forth in SEQ ID NO. 8. In some cases, the mannanase is at least 80%, 85%, 90%, 95%, or at least 99%, identical to a secreted protein sequence encoded by SEQ ID NO. 8. In some cases, the mannanase comprises no more than 1, 2, 4, or 5, single amino acid insertions, substitutions, and/or deletions of a contiguous amino acid region of SEQ ID NO. 8 of at least 25, 50, 100, 125, or 150 amino acids in length.

In some cases, the chitinase comprises at least 25, 50, 100, 125, or 150 contiguous amino acids of, or all of, SEQ ID NO. 9. In some cases, the chitinase comprises no more than 1, 2, 3, 4, or 5 single amino acid substitutions, deletions, and/or additions relative to the protein encoded by SEQ ID NO. 2 or the polypeptide sequence set forth in SEQ ID NO. 9. In some cases, the chitinase is at least 80%, 85%, 90%, 95%, or at least 99%, identical to a secreted protein sequence encoded by SEQ ID NO. 2. In some cases, the chitinase is at least 80%, 85%, 90%, 95%, or at least 99% identical to at least 25, 50, 100, 125, or 150 contiguous amino acids of, or all of, SEQ ID NO. 9. In some cases, the chitinase comprises no more than 1, 2, 4, or 5, single amino acid insertions, substitutions, and/or deletions of a contiguous amino acid region of SEQ ID NO. 9 of at least 25, 50, 100, 125, or 150 amino acids in length.

In some embodiments, a primary component of the recombinant yeast cell wall is β-1,3-glucans, and the cell wall degrading enzyme is or comprises a β-1,3-glucanase. In some embodiments, a primary component of the recombinant yeast cell wall is mannan, and the cell wall degrading enzyme is or comprises a mannanase. In some embodiments, a primary component of the recombinant yeast cell wall is chitin, and the cell wall degrading enzyme is or comprises a chitinase. In some embodiments, expression of a combination of one or two, or more, glucanase cell wall degrading enzymes, such as a β-1-3-glucanase and a β-1-6-glucanase, is induced to permeabilize the cell wall in a regulated manner. In some embodiments, expression of a combination of one or two, or more, chitinase cell wall degrading enzymes is induced to permeabilize the cell wall in a regulated manner. In some embodiments, expression of a combination of one or two, or more, mannanase cell wall degrading enzymes is induced to permeabilize the cell wall in a regulated manner. In some embodiments, expression of a combination of two or more of, or each of, a glucanase, a chitinase, and mannase are induced to permeabilize the cell wall in a regulated manner.

In some embodiments, the cell wall permeabilizing agent is a cell wall degrading enzyme from a yeast that is a natural predator of the host cell. For example, certain spore-forming ascomycetous yeasts of the genera Pichia and Williopsis express cell wall degrading enzymes that exhibit high glucosidic activity against intact S. cerevisiae cell walls. Accordingly, in certain embodiments, the cell wall permeabilizing agent can be a Pichia or Williposis cell wall degrading enzyme. As another example, certain bacteria such as Arthrobacter or Cellulosimicrobium cellulans express cell wall degrading enzymes that exhibit high glucosidic activity against intact S. cerevisiae cell walls. Accordingly, in some embodiments, the cell wall permeabilizing agent is a cell wall degrading enzyme from Arthrobacter or Cellulosimicrobium cellulans.

Inclusion of a nucleic acid sequence that encodes β-glucanase, such as β-1,3-glucanase, is considered particularly advantageous, at least because this particular cell wall degrading enzyme is expected to effectively degrade the yeast cell wall to a sufficient degree to allow the assembled VLP to escape from the recombinant yeast cell.

In addition to, or as an alternative to inclusion of a nucleic acid sequence that encodes a cell wall degrading enzyme, a nucleic acid that encodes a cell wall inhibiting toxin can be included. In these embodiments, the encoded toxin inhibits or prevents formation of cell wall in newly formed recombinant yeast cells. As a result, newly-formed recombinant yeast cells lack a cell wall completely or have only a partially formed cell wall. In either scenario, the therapeutic protein(s) or RNA encoding same included in the recombinant yeast cell are able to leave the newly-formed recombinant yeast cell without the aid of a cell wall degrading enzyme.

If included in a recombinant yeast cell according to an embodiment, a nucleic acid sequence encoding any suitable cell wall inhibiting toxin can be included. Examples of suitable cell wall inhibiting toxins include, but are not limited to, Williopsis Mrakii killer toxin.

The first nucleic acid sequence, accordingly, can comprise any nucleic acid sequence that encodes the regulated promoter selected for the recombinant yeast cell according to a particular embodiment. Similarly, the second nucleic acid sequence can comprise any nucleic acid sequence that encodes the therapeutic protein or proteins selected for the recombinant yeast cell according to a particular embodiment. Lastly, the third nucleic acid sequence can comprise any nucleic acid sequence that encodes a cell wall permeabilizing agent, such as a cell wall degrading enzyme, selected for the recombinant yeast cell according to a particular embodiment.

Expression of the second nucleic acid sequence and the third nucleic acid sequence can be under common genetic control of the regulated promoter. In some embodiments, a genetic construct is made that includes each of the second nucleic acid sequence and the third nucleic acid sequence positioned downstream from the first nucleic acid sequence. In other embodiments, the second nucleic acid sequence is positioned downstream of, and under the genetic control of, a first copy of the first nucleic acid sequence and the third nucleic acid sequence is positioned downstream of, and under the genetic control of, a second copy of the first nucleic acid sequence. In the latter embodiments, the first copy of the first nucleic acid sequence and the second nucleic acid sequence can be positioned on the same or a different nucleic acid molecule (e.g., vector, plasmid, or chromosome) as the second copy of the first nucleic acid sequence and the third nucleic acid sequence. For example, to produce a recombinant yeast cell according to one of these embodiments, a wild type yeast cell can be transformed with two different genetic vectors—a first genetic vector that encodes the first copy of the first nucleic acid sequence and the second nucleic acid sequence, and a second genetic vector that encodes the second copy of the first nucleic acid sequence and the third nucleic acid sequence.

In some embodiments, the recombinant yeast cell further comprises a fourth nucleic acid sequence encoding at least one viral or self-assembling peptide structural element for purposes of forming a VLP. In certain embodiments, at least one viral structural element is preferably linked to a nucleic acid binding protein via suitable flexible linker so as to avoid steric hindrance. In an exemplary embodiment, the linker comprises the sequence STSSEFCSRRYRGPGIHRPVAT (SEQ ID NO: 33). In these embodiments, the second nucleic acid sequence encoding the therapeutic protein includes in its mRNA sequence a stem loop recognition site that corresponds to the mRNA binding peptide referenced above. Similar to the first, second, and third nucleic acid sequences, the fourth nucleic acid sequence in these embodiments can comprise a nucleic acid sequence that does not occur naturally in the wild-type yeast cell and that has been artificially introduced into the wild-type yeast cell to produce recombinant yeast cell.

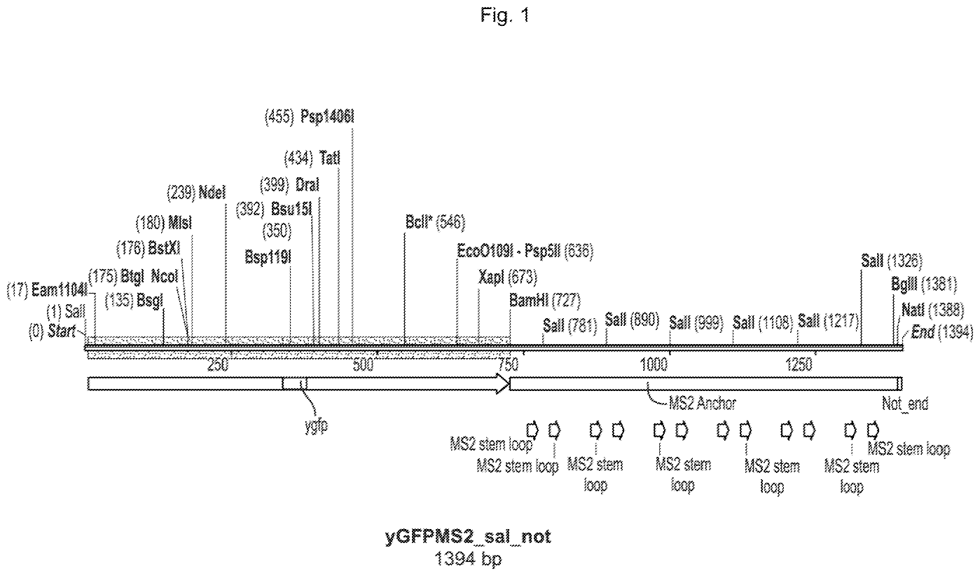

In one particular example in accordance with these embodiments, the fourth nucleic acid sequence encodes a GAG-MS2 fusion protein. The GAG protein portion of the fusion protein is a protein that assembles to form viral particles while the MS2 portion of the fusion protein is an MS2 bacteriophage coat protein that naturally interacts with well-defined non-translated stem loop structures in RNA. For illustrative purposes, an example of a nucleic acid sequence for the second nucleic acid sequence comprises SEQ ID NO. 10. An example precursor to a suitable nucleic acid sequence for the second nucleic acid sequence in these embodiments comprises SEQ ID NO. 11, which is schematically illustrated in FIG. 1. SEQ ID NO. 11 encodes the MS2 anchor, which includes a series of stem loop structures that MS2 protein can bind, and ygfp, which includes a series of well-characterized restriction enzyme sites that can be used for insertion of one or more sequences encoding therapeutic proteins of interest. Accordingly, the sequence encoding the GFP reporter can be substituted for a nucleic acid sequence encoding any one of the therapeutic proteins described herein.

In some cases, the MS2 binding sequence can be a part of a repeated array of MS2 sequences. In some cases, the repeated MS2 sequences can compromise genetic stability of the recombinant coding sequence. In one embodiment, the nucleic acid binding sites in the repeated array are synonymous binding sites that are different in sequence and yet retain the cognate protein binding function. Such arrays of synonymous nucleic acid binding sites are described in, e.g., Wu et al., Genes Dev. 2015 Apr. 15 (29(8); 876-886. In some embodiments the MS2 sequence comprises a following hairpin loop forming sequence of SEQ ID NO. 12 (NRNDSASSANCASSSNNYN), wherein S represents C or G; D represents A, G, or U; R represents A or G; and Y represents C or U. In some embodiments, the nucleic acid binding sites are in a repeated array comprising from 8 to 48 iterations of an MS2 sequence, such as an MS2 sequence of SEQ ID NO. 12. In some embodiments, the repeated array comprises from 8 to 24, preferably 24 iterations of an MS2 sequence, such as an MS sequence of SEQ ID NO. 12. In some cases, the repeated array of nucleic acid binding sites is encoded by SEQ ID NO. 13.

These embodiments are considered particularly advantageous at least because the (e.g., Gag)-MS2 fusion protein works to bind and package the RNA corresponding to the therapeutic protein encoded by the second nucleic acid sequence. In use, a recombinant yeast cell according to one of these embodiments will release VLPs that include RNA that encodes the therapeutic protein of interest. If included in a pharmaceutical composition or a food composition according to an embodiment, for example, the recombinant yeast cell will release VLPs that are ingested by enterocytes or other gut cells within the animal ingesting the pharmaceutical composition or food composition. These cells can then translate the RNA and express the therapeutic protein(s) in the normal functioning of the animal's protein expression system.

It will be appreciated that the GAG protein encoding sequence can be substituted with a variety of VLP-forming protein sequences, including but not limited to a sequence encoding an influenza matrix protein, a coronaviral capsid protein, a GAG-homology protein and the like. In some embodiments, the GAG-homology protein can be selected from Arc, ASPRV1, a Sushi-Class protein, a SCAN protein, or a PNMA protein. In embodiments, the GAG-homology protein is a PNMA protein, e.g., ZCC18, ZCH12, PNM8B, PNM6A, PNMA6E_i2, PMA6F, PMAGE, PNMA1, PNMA2, PNM8A, PNM8B, PNMA3, PNMA4, PNMA5, PNMA6, PNMA7, MOAP1, or CCD8. In embodiments, the GAG-homology protein is an Arc protein, e.g., hARC or dARC1. In embodiments, the GAG-homology protein can comprise ASPRV1. In some embodiments, the GAG-homology protein is PEG10, RTL3, RTL10, or RTL1. In some embodiments, the PEG10 GAG homology protein is a PEG10_i6 or a PEG10_i2. In certain embodiments, the GAG homology protein is a SCAN protein, for example PGBD1.

In these embodiments, the fourth nucleic acid sequence can be, but need not be, under common genetic control of the regulated promoter along with the first, second, and/or third nucleic acid sequences.

VLPs, including neVLPs and/or eVLPs can be engineered to include an amplifiable replicon, or constructs encoding such a replicon. As used herein, an “amplifiable replicon” comprises minimal nucleic acid sequence(s) capable of supporting self-replication in a host cell. For example, a VLP can package an RNA nucleic acid that encodes an RNA-dependent RNA polymerase (RdRp) capable of replicating the packaged RNA nucleic acid or portion thereof at least 1, preferably at least 2 times. In some cases, the packaged nucleic acid includes a 5′ and/or a 3′ untranslated region (UTR), preferably the packaged nucleic acid includes a 5′ and a 3′ UTR. Typically, the amplifiable replicon contains a gene of interest, such as nucleic acid sequence encoding a therapeutic protein.

In some embodiments, such amplifiable replicons can be constructed from portions of a parainfluenza virus (PIV) genome, such as a PIV type 5 (e.g., PIV5) genome. In some embodiments, the amplifiable replicon is a nucleic acid comprising, all, a functional portion of, or at least a portion of, a parainfluenza virus (e.g., PIV5) NP, V/P, and L gene, and optionally a gene of interest, such as nucleic acid sequence encoding a therapeutic protein, an MS2 protein, an MS2 binding site, and/or a reporter. In some embodiments, the amplifiable replicon lacks one or more PIV genes (e.g., PIV5 genes) selected from the group consisting of M, F, SH and HN, or is incapable of expressing one or more of the PIV5 proteins selected from the group consisting of M, F, SH and HN. In some cases, the amplifiable replicon comprises PIV5 NP, V/P and L genes. In some cases, the amplifiable replicon comprises a gene of interest inserted between a PIV (e.g., PIV5) V/P and L gene.

Suitable PIV-based replicons include, but are not limited to, those replicons described in Wei et al., npj Vaccines 2, 32 (2017), preferably wherein said replicons include a nucleic acid encoding a therapeutic protein (e.g., an antibody or fragment thereof), reporter, and/or other gene of interest as described herein, an MS2 or other anchor sequence as described herein, an MS2 protein as described herein, or a VLP-forming polypeptide as described herein (e.g., matrix, GAG, or capsid protein, GAG-homology protein, envelope protein, functional fragments thereof, or combination thereof), between a 5′ and 3′ UTR. Such replicons can include, or be used in conjunction with other genetic elements, such as promoters or cis- or trans-acting helper polypeptides or genes, that are essential for supporting self-replication, as described in Wei et al., U.S. Pat. No. 9,034,343, and/or WO 2002/077211, or an orthologue thereof, such as an ortholog of an element or polypeptide of U.S. Pat. No. 9,034,343, that is disclosed in WO 2002/077211.

In an exemplary embodiment a replicon is generated by replacing PIV5 fusion glycoprotein (e.g., SEQ ID NO. 14), M, SH, and/or HN with a gene of interest, optionally wherein the replicon further includes a selectable marker (e.g., a hygromycin resistance marker), preferably wherein the selectable marker is inserted between V/P and L.

In an exemplary embodiment, the PIV5 L gene encodes a protein comprising SEQ ID NO. 15. In some cases, the PIV5 L gene encodes a protein comprising at least 25, 50, 100, 125, or 150 contiguous amino acids of, or all of, SEQ ID NO. 15. In some cases, the PIV5 L gene encodes a protein that is at least 80%, 85%, 90%, 95%, or 99% identical to at least 25, 50, 100, 125, or 150 contiguous amino acids of, or all of, SEQ ID NO. 15. In some cases, the PIV5 L gene encodes a protein comprising no more than 1, 2, 4, or 5, single amino acid insertions, substitutions, and/or deletions of SEQ ID NO. 15. In some cases, the PIV5 L gene encodes a protein comprising no more than 1, 2, 4, or 5, single amino acid insertions, substitutions, and/or deletions of a contiguous amino acid region of SEQ ID NO. 15 of at least 25, 50, 100, 125, or 150 amino acids in length.

In an exemplary embodiment, the PIV5 NP gene encodes a protein comprising SEQ ID NO. 16. In some cases, the PIV5 NP gene encodes a protein comprising at least 25, 50, 100, 125, or 150 contiguous amino acids of, or all of, SEQ ID NO. 16. In some cases, the PIV5 NP gene encodes a protein that is at least 80%, 85%, 90%, 95%, or 99% identical to at least 25, 50, 100, 125, or 150 contiguous amino acids of, or all of, SEQ ID NO. 16. In some cases, the PIV5 NP gene encodes a protein comprising no more than 1, 2, 4, or 5, single amino acid insertions, substitutions, and/or deletions of SEQ ID NO. 16. In some cases, the PIV5 NP gene encodes a protein comprising no more than 1, 2, 4, or 5, single amino acid insertions, substitutions, and/or deletions of a contiguous amino acid region of SEQ ID NO. 16 of at least 25, 50, 100, 125, or 150 amino acids in length.

In an exemplary embodiment, the PIV5 V/P gene encodes a protein comprising SEQ ID NO. 17. In some cases, the PIV5 V/P gene encodes a protein comprising at least 25, 50, 100, 125, or 150 contiguous amino acids of, or all of, SEQ ID NO. 17. In some cases, the PIV5 V/P gene encodes a protein that is at least 80%, 85%, 90%, 95%, or 99% identical to at least 25, 50, 100, 125, or 150 contiguous amino acids of, or all of, SEQ ID NO. 17. In some cases, the PIV5 V/P gene encodes a protein comprising no more than 1, 2, 4, or 5, single amino acid insertions, substitutions, and/or deletions of SEQ ID NO. 17. In some cases, the PIV5 V/P gene encodes a protein comprising no more than 1, 2, 4, or 5, single amino acid insertions, substitutions, and/or deletions of a contiguous amino acid region of SEQ ID NO. 17 of at least 25, 50, 100, 125, or 150 amino acids in length.

In some cases, one or all of the M, F, SH and HN PIV (e.g., PIV5) genes are replaced with the gene of interest. In some cases, one or more of NP, or V/P, are replaced with a therapeutic protein gene. In some embodiments, the amplifiable replicon comprise a PIV (e.g., PIV5) 5′ and/or 3′ UTR. In some embodiments, the gene of interest and the RdRp gene are between the 5′ and 3′ UTRs. In some embodiments, the RdRp gene and the gene of interest are encoded as a single polypeptide that includes a self-cleaving peptide sequence between RdRp protein and the protein encoded by the gene of interest. In some cases, the self-cleaving peptide is a 2A self-cleaving peptide, such as a T2A peptide of Thosea asigna virus. Additional embodiments of amplifiable PIV5 replicons are described in, e.g., WO 2016/176510.

In some embodiments, an amplifiable replicon comprises a functional fragment, or all, of an RdRp gene from Nodamura virus (NoV). See, e.g., Biddlecome et al., PLoS One. 2019; 14(6): e0215031. In some cases, the amplifiable replicon comprises a NoV RdRp gene or functional fragment thereof and a gene of interest between, 5′ and 3′ UTRs of NoV RNA1. In some embodiments, the RdRp gene and the gene of interest are encoded as a single polypeptide that includes a self-cleaving peptide sequence between RdRp protein and the protein encoded by the gene of interest. In some cases, the self-cleaving peptide is a 2A self-cleaving peptide, such as a T2A peptide of Thosea asigna virus.

In some embodiments, an amplifiable replicon comprises a gene encoding a therapeutic protein of interest and functional fragment, or all, of an alphaviral replicase. Alphaviruses encode four nonstructural proteins (nsP1-4), initially produced as a polyprotein P1234. nsP4 is the core RNA-dependent RNA polymerase but all four nsPs, or at least functional fragments thereof, are required for RNA synthesis. In some embodiments, an amplifiable replicon comprising a functional fragment, or all, of an alphaviral replicase, further comprises an alphaviral 5′ cis-acting element and/or a 3′ UTR, preferably a 5′ cis-acting element and a 3′ UTR. Suitable alphavirus replicon embodiments include, but are not limited to, those described in U.S. 2006/0198854, by Pushko, preferably wherein said replicons include a nucleic acid encoding an immunogen (e.g., hemagluttinin, neuraminidase, or spike protein, or, reporter, and/or other gene of interest as described herein, an MS2 or other anchor sequence as described herein, an MS2 protein as described herein, or a VLP-forming polypeptide as described herein (e.g., matrix, GAG, or capsid protein, GAG-homology protein, envelope protein, or functional fragments thereof, or combination thereof), between the 5′ cis-acting element (e.g., 5′ UTR) and 3′ end of the replicon (e.g., 3′ UTR). Such replicons can include, or be used in conjunction with other genetic elements, such as promoters or cis- or trans-acting helper polypeptides or genetic elements, that are essential for supporting self-replication, as described in U.S. 2006/0198854.

Amplifiable replicon embodiments described herein, including those comprising one or more PIV genes and/or one or more replicase or RdRp genes (e.g., PIV5 or NoV RdRp or alphaviral replicase) can be packaged into any one of the VLPs described herein. Similarly, amplifiable replicons can be produced and packaged in any one of the yeast host cell systems described herein and released as VLPs from a permeabilized yeast host cell.

In one particular example in accordance with these embodiments, the second nucleic acid sequence 112 comprises at least a portion of each of Parainfluenza 5 (PIV5) NP, V/P, and L genes. In this example, the second nucleic acid sequence 112 lacks one or more of the PIV5 genes selected from the group consisting of M, F, SH, and HN.

In these embodiments, the fourth nucleic acid sequence can be, but need not be, under common genetic control of the regulated promoter along with the first, second, and third nucleic acid sequences. Also in these embodiments, the second nucleic acid sequence can encode suitable therapeutic protein(s) of interest. Suitable examples include an antibody or fragment thereof, or a hormone.

Following transformation, a recombinant yeast cell according to an embodiment can be treated further using any desirable and/or suitable techniques, processes, and/or methods based on a desired outcome, characteristic, or property. For example, as described in detail below, the recombinant yeast cell can be used in pharmaceutical compositions. For these embodiments, the inventors have determined that dehydrated recombinant yeast cells are particularly advantageous. Accordingly, recombinant yeast cells according to a particular embodiment can be processed using conventional methods for dehydrating yeast, such as freeze-drying. Freeze-drying the recombinant yeast cell is considered particularly advantageous as the resulting freeze-dried recombinant yeast cell has a desirable residual moisture level and long-term stability. Accordingly, in some embodiments, the recombinant yeast cell comprises a freeze-dried recombinant yeast cell. Furthermore, in some embodiments, the recombinant yeast cell is microencapsulated and baked in food or placed in liquid.

In certain embodiments, the ingestible vessel can comprise any suitable ingestible vessel and a skilled artisan will be able to select an appropriate ingestible vessel for inclusion in a therapeutic protein composition according to a particular embodiment based on various considerations, including the nature and quantity of the recombinant yeast cells included in the therapeutic protein compositions, any storage and handling requirements, and other considerations. Examples of suitable ingestible vessels include, but are not limited to, capsules, acid-resistant capsules, and capsules defining pores.

The at least one recombinant yeast cell of the present invention can comprise any recombinant yeast cell according to an embodiment of the invention, including the example recombinant yeast cells described herein. Furthermore, the at least one recombinant yeast cell can comprise any suitable number of recombinant yeast cells, and a skilled artisan will be able to select an appropriate number of recombinant yeast cells for inclusion in a therapeutic protein composition according to a particular embodiment based on various considerations, including the nature of the protein included in the recombinant yeast cell, the copy number of the protein included in the recombinant yeast cell, and other considerations. Examples of suitable numbers of recombinant yeast cells for inclusion in a therapeutic protein composition according to an embodiment of the invention include, but are not limited to, one, at least one, more than one, two, a plurality, three, four, five, six, seven, eight, nine, ten, more than ten, one hundred, at least one hundred, more than one hundred, one thousand, at least one thousand, more than one thousand, one million, at least one million, and more than one million. Examples of suitable ranges of numbers of recombinant yeast cells for inclusion in a therapeutic protein composition according to an embodiment of the invention include, but are not limited to, between about 1 and about 10⁷, between about 1 and about 10⁶, between about 1 and about 10⁵, between about 1 and about 10⁴, between about 1 and about 10³, between about 1 and about 10², and between about 1 and about 10.

The food composition of the present invention comprises a pharmaceutical composition according to an embodiment of the invention and at least one foodstuff. Thus, the pharmaceutical composition comprises an ingestible vessel defining a cavity and at least one recombinant yeast cell according to an embodiment disposed in the cavity. The ingestible vessel may comprise a polymeric shell that has been sprayed onto a plurality of recombinant yeast cells to microencapsulate the recombinant yeast cells in the cavity defined by the ingestible vessel formed by the polymeric shell.

The food composition includes at least one pharmaceutical composition and more than one pharmaceutical composition can be included. Indeed, any suitable number of pharmaceutical compositions can be included in a food composition according to a particular embodiment, and a skilled artisan will be able to select a suitable number for inclusion according to a particular embodiment based on various considerations, including the size, shape, and configuration of the food composition, the nature of the pharmaceutical composition, including the number of recombinant yeast cells included in each pharmaceutical composition included in the food composition, and other considerations. Examples of suitable numbers of pharmaceutical compositions that can be included in a food composition according to an embodiment of the invention include, but are not limited to, one, at least one, more than one, two, a plurality, three, four, five, six, seven, eight, nine, ten, more than ten, between about 1 and about 10⁷, between about 1 and about 10⁶, between about 1 and about 10⁵, between about 1 and about 10⁴, between about 1 and about 10³, between about 1 and about 10², and between about 1 and about 10.

The at least one foodstuff of the present invention can comprise any substance considered suitable for consumption as food by an animal. Examples include flour, wheat, sugar, butter, bread, dough, meat, yogurt, a fruit or portion thereof, a vegetable or portion thereof, water or another liquid, and combinations of these examples.

The food composition can take any suitable form, including, but not limited to, a cookie, a candy, a bar, a cracker, a wafer, a loaf, a beverage, a yogurt, and any other form considered desirable.

Thus, encompassed by the disclosure herein is a method of administering a therapeutic protein to a subject, where the method comprises providing a pharmaceutical composition as herein disclosed, and orally administering the pharmaceutical composition to the subject.

Furthermore, the present disclosure encompasses methods of treating a subject suffering from a disease or condition. For example, a method of treating a subject comprises providing a pharmaceutical composition as herein disclosed, and orally administering the pharmaceutical composition to the subject, thereby administering to the subject a therapeutic protein that functions to decrease one or more signs or symptoms associated with the disease or condition.

Also within the scope of the present disclosure are kits. In one embodiment, a kit comprises a packaging substrate, a pharmaceutical composition according to an embodiment herein, and instructions for using the pharmaceutical composition (i.e., instructions for orally delivering the pharmaceutical composition to an animal, instructions for orally ingesting the pharmaceutical composition, or both). In another embodiment, a kit comprises a packaging substrate, a food composition according to an embodiment herein, and instructions for using the food composition (i.e., instructions for orally delivering the food composition to an animal, instructions for orally ingesting the food composition, or both).

The recombinant yeast cells of the present invention can be produced using conventional means. An initial step comprises creating a recombinant yeast cell by introducing into a wild-type yeast cell a first nucleic acid sequence encoding a regulated promoter, a second nucleic acid sequence encoding an protein, and a third nucleic acid sequence encoding a cell wall permeabilizing agent (e.g., cell wall degrading enzyme). The recombinant yeast cell can comprise any recombinant yeast cell according to an embodiment. Thus at least one, or each, of the first nucleic acid sequence, second nucleic acid sequence, and third nucleic acid sequences can comprise a nucleic acid sequence that does not occur naturally in the wild-type yeast cell from which the recombinant yeast cell is derived. In some embodiments, expression of the second and third nucleic acid sequences is under common genetic control of the regulated promoter. The introducing step can be performed in accordance with any suitable technique or method, including conventional transformation techniques and methods.

Another step comprises disposing the recombinant yeast cell in a cavity defined by an ingestible vessel to produce a therapeutic protein composition in accordance with an embodiment.

In further embodiments, the initial step comprises creating a recombinant yeast cell by introducing into a wild-type yeast cell a first nucleic acid sequence encoding a positive repressible promoter that is repressed in the presence of a repressor, a second nucleic acid sequence encoding a therapeutic protein, and a third nucleic acid sequence encoding a cell wall permeabilizing agent (e.g., cell wall degrading enzyme). The recombinant yeast cell can comprise any recombinant yeast cell according to an embodiment. Thus at least one, or each, of the first nucleic acid sequence, second nucleic acid sequence, and third nucleic acid sequences can comprise a nucleic acid sequence that does not occur naturally in the wild-type yeast cell from which the recombinant yeast cell is derived. Also, in some cases, expression of the second and third nucleic acid sequences is under common genetic control of the regulated promoter. The introducing step can be performed in accordance with any suitable technique or method, including conventional transformation techniques and methods.

The positive repressible promoter can comprise any suitable positive repressible promoter. As described above, the inventors have determined that the Tet-off promoter is considered advantageous. In these embodiments, the repressor comprises tetracycline or a tetracycline derivative.

Another step comprises growing a plurality of recombinant yeast cells derived from the recombinant yeast cell in a culture comprising the repressor.

Another step comprises removing the repressor from the culture.

Another step comprises disposing the plurality of recombinant yeast cells in a cavity defined by an ingestible vessel;

An optional step comprises allowing a pre-defined period of time to pass between the step of removing the repressor from the culture and the step of disposing the plurality of recombinant yeast cells in a cavity defined by an ingestible vessel. Inclusion of this optional step is considered advantageous at least because it enables activation of the promoter and, as a result, expression of the second and third nucleic acid sequences for a period of time before disposing the plurality of recombinant yeast cells in a cavity defined by an ingestible vessel.

Another optional step comprises freeze-drying the plurality of recombinant yeast cells. If included, this step can be performed before, concurrently with, or after the step of disposing the plurality of recombinant yeast cells in a cavity defined by an ingestible vessel.

### EXAMPLES

### Example 1: HIV GAG-MS2 eVLP Production

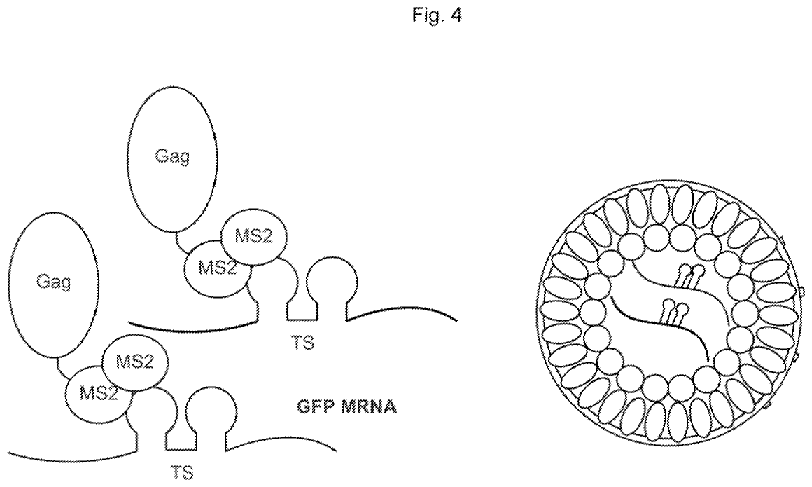

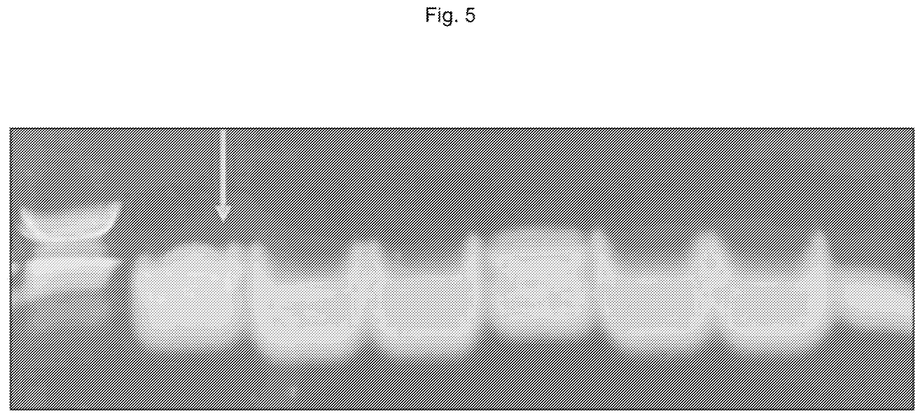

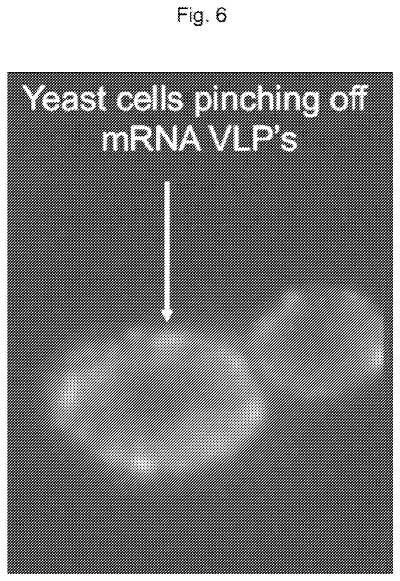

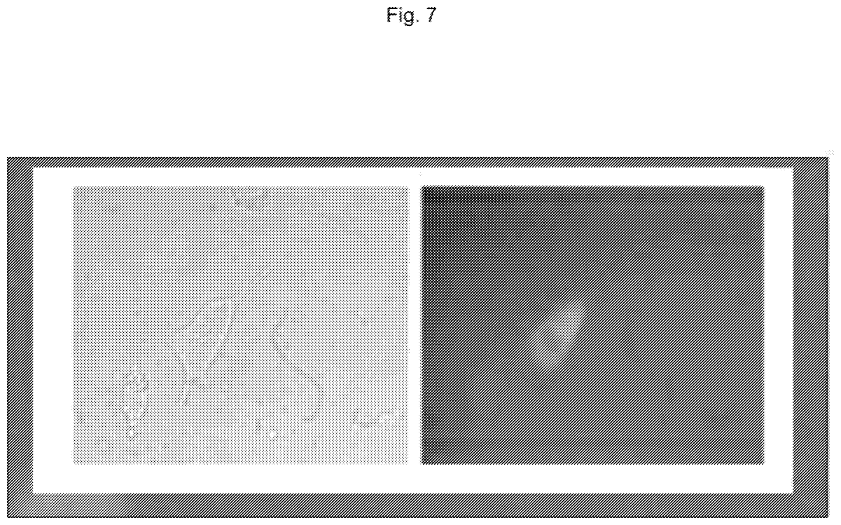

FIG. 4 illustrates a schematic diagram of eVLP encoded by the constructs produced in this example. Yeast cells were transformed with nucleic acid encoding HIV-GAG fusion protein and EGFP-MS2 mRNA, cultured in shake flasks to approximately OD₆₀₀=10 and induced. Recombinant cells were cultured under inducing conditions and yeast cells were harvested from the culture medium after induction. Culture medium was also harvested to obtain eVLP. RT-PCR analysis confirmed the presence of EGFP mRNA in the eVLP (FIG. 5). Microscopy of recovered yeast cells demonstrates secretion of eVLPs (FIG. 6). eVLPs were purified and incubated with dendritic cells (DCs). After 24 hours incubation with DCs, fluorescence microscopy confirmed EGFP production in the DCs (FIG. 7). These results indicate that the DCs phagocytosed the eVLPs and translated the EGFP mRNA for expression of functional EGFP.

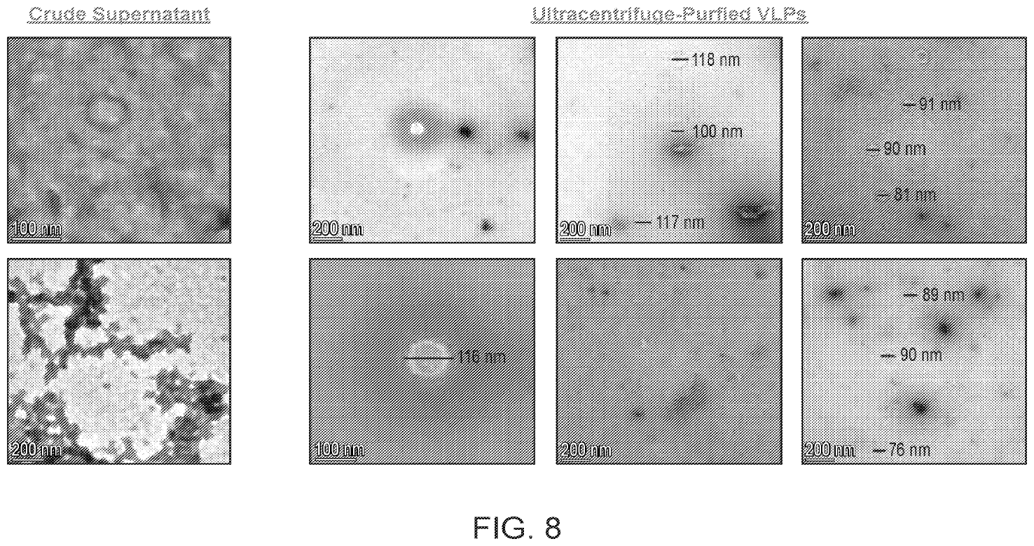

eVLPs were also analyzed by transmission electron microscopy as crude culture supernatant (FIG. 8, left) and after ultracentrifugation to obtain purified eVLPs (FIG. 8, right). The purified eVLPs were approximately 80 to 120 nm in diameter with an envelope thickness of about 4.2 nm.

### Example 2: OCH1 Mutant Strain with Inserted Recombinase

The following Example describes the preparation of the yeast BY4741 OCH1—mutant strain. As described in detail below, homologous recombination was used to insert Met15 and Cre recombinase genes into the genomic OCH1 open reading frame. The insertion knocked out OCH1, added an auxotrophic marker, methionine, for selection, and inserted the Cre recombinase gene.

Materials and Methods

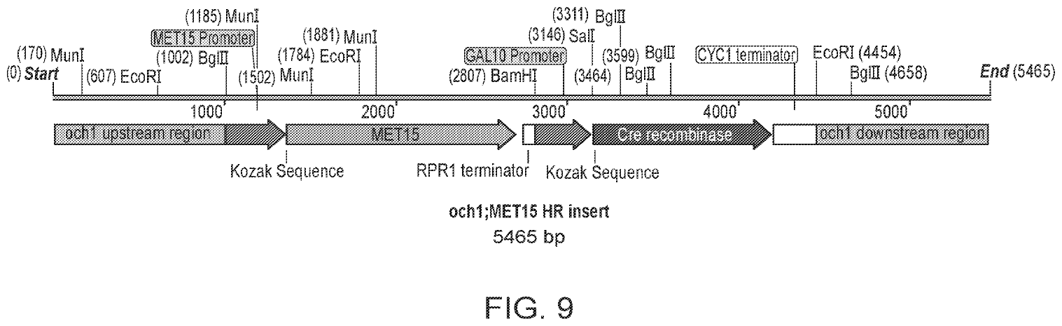

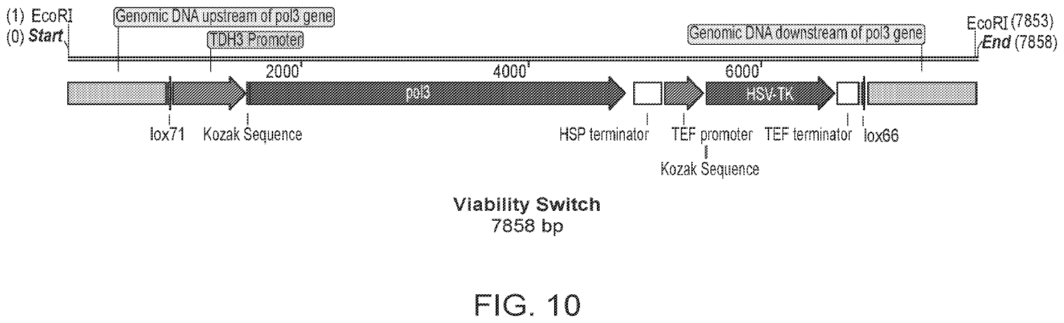

Plasmids VB200809-1175ufz (pOCH) and VB200806-2136scs (pVS) were transformed into NEB DH5a cells and glycerol stocks were made. Plasmid DNA was isolated from pOCH transformants, and digested with KpnI to linearize, leaving 1000 bp of homologous sequence to the genomic OCH1 at both ends of the linearized DNA. The linearized construct is shown in FIG. 9. The sequence of the insert is provided as SEQ ID NO: 18. Cre recombinase can be used, e.g., to delete a gene essential to yeast replication (e.g., DNA polymerase). Yeast cells in which the gene essential to replication is not deleted may express a cytotoxic agent (see FIG. 10).

Cells were transformed using LiAc/SS carrier DNA/PEG, (Gietz, R. D., et al. (2007) Nature Protocols, Vol. 2 No. 1 31-35) and Electroporation. Once transformed into yeast strain BY4741, which are auxotrophic for methionine, the yeast integrate the linearized construct into the genome by homologous recombination with the OCH1 gene sequence located on the chromosome.

Resulting transformants were plated on selective media, 2% YNB-Met, and grown at 30° C. After 14 days colonies were streaked for isolation on fresh 2% YNB-Met plates and again grown at 30° C. After sufficient growth (˜5-7 days) isolated colonies were used to inoculate 2% YNB-Met broth, and were grown shaking at 30° C. Cells were spun down at 500×g, supernatant was discarded, and pellets were resuspended in fresh media every 48 hours.

To confirm that the construct had integrated into the yeast genome, genomic DNA was isolated from three transformants, and that DNA was used as template for PCR. Four sets of primers were designed to confirm integration, as well as to rule out episomal expression, as shown below.

Primer Sequences

| | Amp-F: |
|---|---|
| (SEQ ID NO: 19) | |
| | TTATCCGCCTCCATCCAGTC |
| | Amp-R: |
| (SEQ ID NO: 20) | |
| | CTGCGGCCAACTTACTTCTG |
| | Cre-F: |
| (SEQ ID NO: 21) | |
| | ccagctttgccagttgatgcaac |
| | Cre-R: |
| (SEQ ID NO: 22) | |
| | gctctagccatatctctagcggcac |
| | OCHMET-F: |
| (SEQ ID NO: 23) | |
| | ggctgggcctcaactaaacg |
| | OCHMET-R: |
| (SEQ ID NO: 24) | |
| | cgatgttgtcaccagtgtgtgcc |
| | OCHCRE-F: |
| (SEQ ID NO: 25) | |
| | gtgccgctagagatatggctagagc |
| | OCHCRE-R: |
| (SEQ ID NO: 26) | |
| | ctgctgcaagtgcgacaacc |

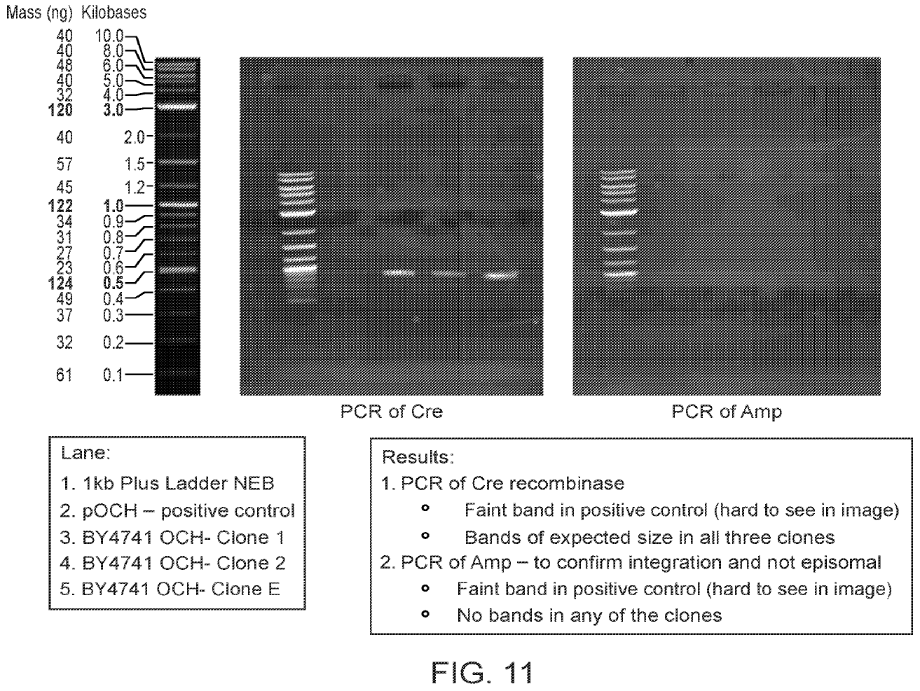

Preliminary PCR data using the Cre and Amp primers indicated that all three transformants tested integrated the construct into the genome (FIG. 11).

### Example 3: Inhibition of Protein Expression in Yeast

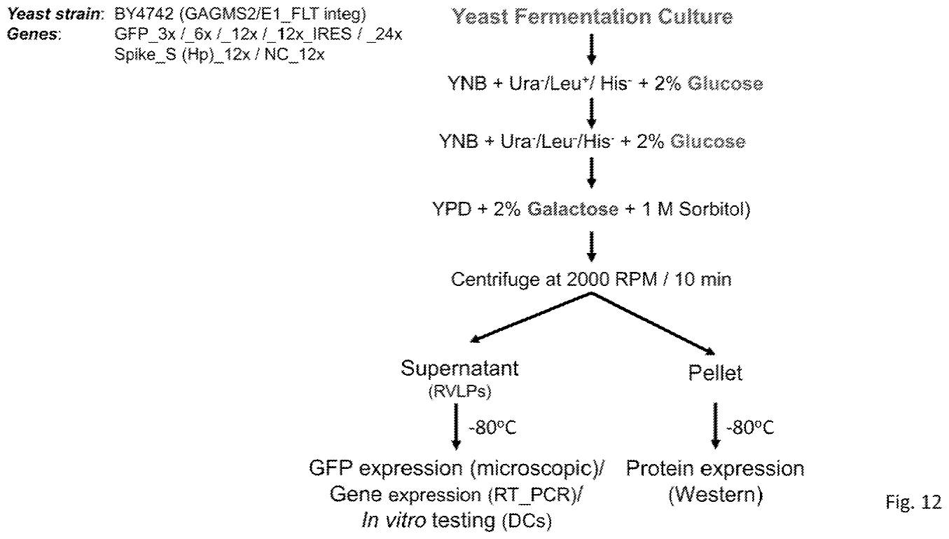

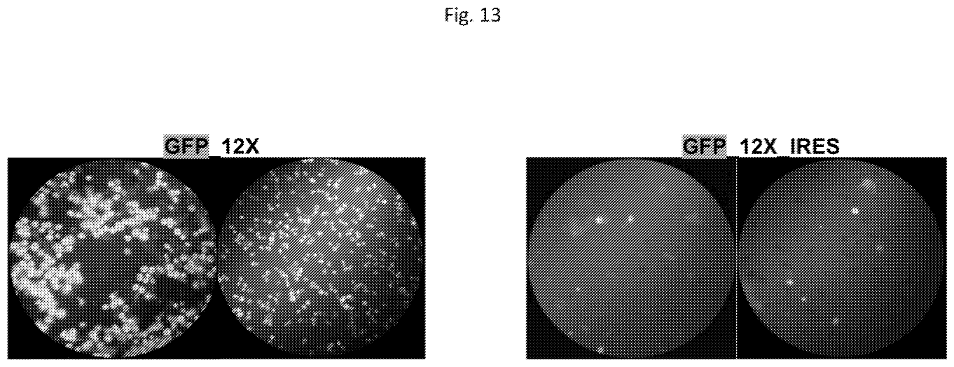

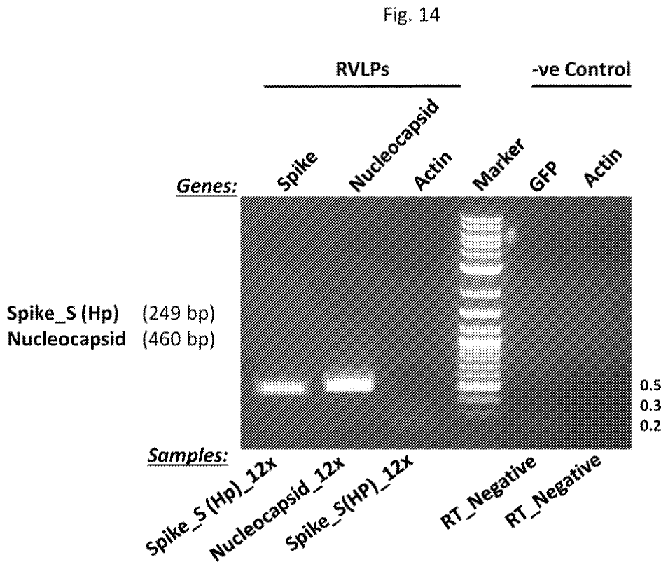

FIG. 12 illustrates the protocol followed for demonstrating inhibition of target protein expression in yeast cells via insertion of an IRES element as disclosed herein. FIG. 13 illustrates the reduction (right) in protein expression in recombinant yeast comprising an IRES element upstream of the coding sequence for the target gene, as compared to protein expression in absence of the IRES element (left). Gene-specific RT-PCR products were obtained from the VLPs containing mRNA of gene of interest using gene specific primers, as illustrated in FIG. 14.

### Example 4: C. difficile Nanobody

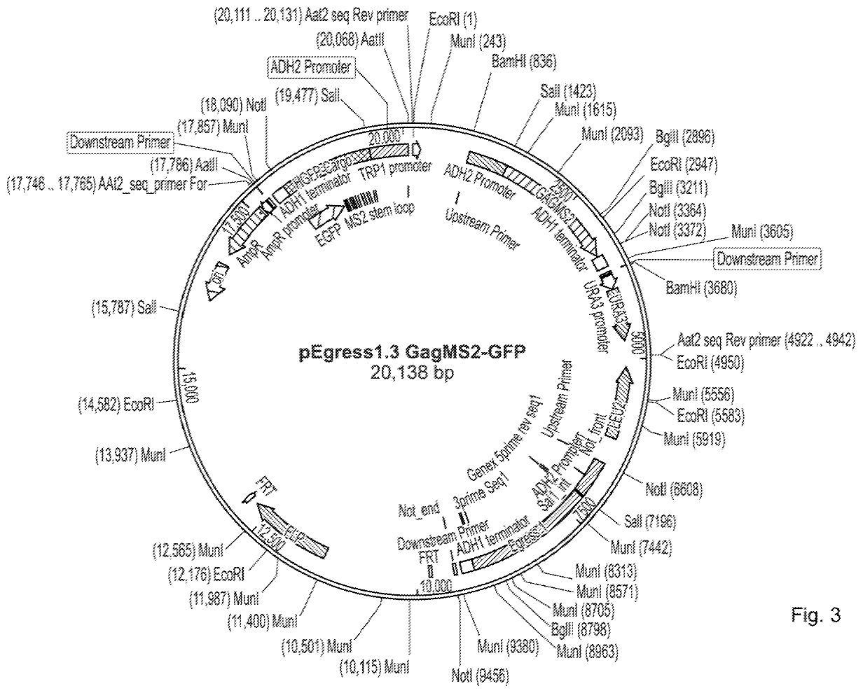

A nanobody directed against surface layer protein (SLP) of C. difficile (see, for example, Kandalaft et al., Targeting surface-layer proteins with single-domain antibodies: a potential therapeutic approach against Clostridium difficile-associated disease. App Microbiol Biotechnol (2015) 99:8549-8562. DOI 10.1007/s00253-015-6594-1) can be used in accordance with the present disclosure. Specifically, a construct encoding the C. difficile nanobody can be cloned, e.g., into a tricassette containing GAGMS2 and Egress1 with the 12× stem loop for making virus-like particles containing mRNA of the CR3022 neutralizing antibody (see construct illustrated at FIG. 3).

### Example 5: COVID-19 Spike Protein Nanobody Production

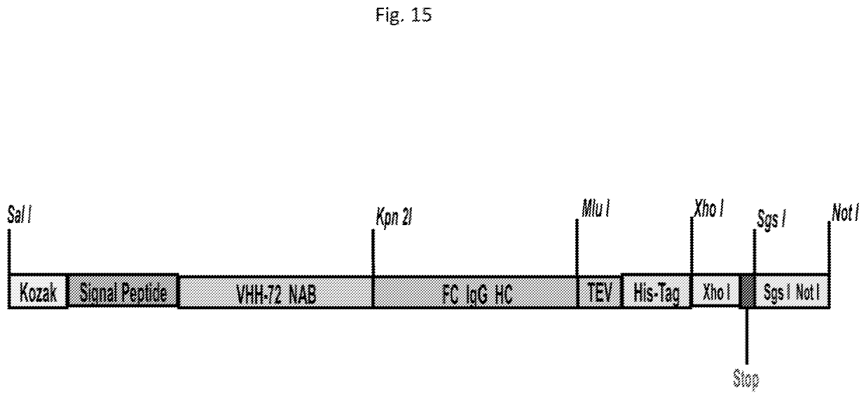

A VHH-72 nanobody construct is fused with human IgG (FIG. 15) in order to produce effective binding and neutralization capacity (see, for example, Wrapp et al., (2020) Structural basis for potent neutralization of betacoronoviruses by single-domain camelid antibodies. Cell 181: 1004-1015.).

Nucleic acid sequence: GenBank: MT350284.1—SARS VHH-72 sequence (SEQ ID NO. 27)

| | |
|---|---|
| 01 | caggtgcagc tgcaggagtc tgggggagga ttggtgcagg |
| | ctgggggctc tctgagactc |
| 61 | tcctgtgccg cctctggacg caccttcagt gaatatgcca |
| | tgggctggtt ccgccaggct |
| 121 | ccagggaagg aacgtgagtt tgtagcaact attagctgga |
| | gtggtggtag cacatactat |
| 181 | acagactccg tgaagggccg attcaccatc tccagagaca |
| | acgccaagaa cacggtgtat |
| 241 | ctgcaaatga acagcctgaa acctgatgac acggccgttt |
| | attactgtgc agcagccggg |
| 301 | ttaggtacgg tagtatcgga gtgggattat gactatgact |
| | actggggcca ggggacccag |
| 361 | gtcaccgtct cctca |

The VHH72-Fc nanobody efficiently neutralizes SARS-CoV-1 and SARS-CoV-2 pseudoviruses. The VHH72-FC nanobody exhibits strong affinity for SARS-CoV-2 RBD is ELISA assays. The VHH72-FC nanobody construct can be cloned, e.g., into a tricassette containing GAGMS2 and Egress1 with the 12× stem loop for making virus-like particles containing mRNA of the VHH-72 nanobody (see construct illustrated at FIG. 3).

### Example 6: CR3022 Monoclonal Antibody for Covid-19 Spike Protein

CR3022, a neutralizing antibody previously isolated from a convalescent SARS patient, in complex with the receptor binding domain (RBD) of the SARS-CoV-2 spike (S) protein. CR3022 targets a highly conserved epitope, distal from the receptor binding site, that enables cross-reactive binding between SARS-CoV-2 and SARS-CoV. Structural modeling further demonstrates that the binding epitope can only be accessed by CR3022 when at least two RBDs on the trimeric S protein are in the “up” conformation and slightly rotated. These results provide molecular insights into antibody recognition of SARS-CoV-2.

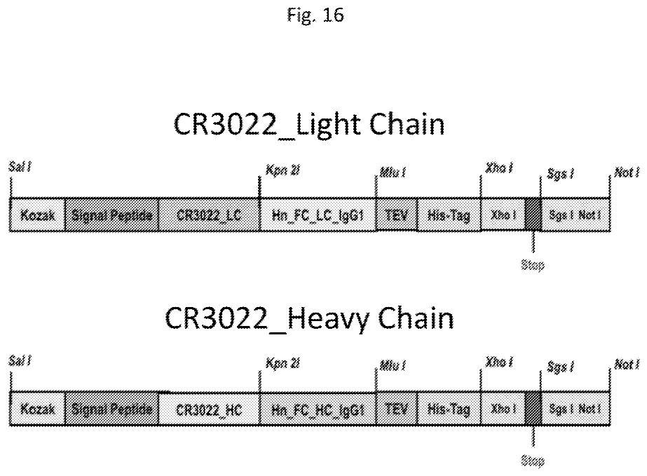

The gene construct of SARS-CoV-2 spike monoclonal antibody (CR3022) (FIG. 16) is humanized by fusion of the CR3022 heavy chain and light chain monoclonal antibody with human IgG in order to produce effective binding and neutralization capacity (A highly conserved cryptic epitope in the receptor-binding domains of SARS-CoV-2 and SARS-CoV. Meng Yuan, Nicholas C. Wu, Xueyong Zhu, Chang-Chun D. Lee, Ray T. Y. So, Huibin Lv, Chris K. P. Mok and Ian A. Wilson. Science Vol. 368, Issue 6491:630-633, 2020. DOI: 10.1126/science.abb7269). The constructs can be cloned, e.g., into a tricassette containing GAG-MS2 and Egress1 with the 12× stem loop for making virus-like particles containing mRNA of the CR3022 neutralizing antibody (see construct illustrated at FIG. 3)

The nucleic acid sequences for CR3022 light and heavy chains are:

Nucleic acid sequence of CR3022_LC
(SEQ ID NO. 28):
gacatccagttgacccagtctccagactccctggctgtgtctctgggcga
gagggccaccatcaactgcaagtccagccagagtgttttatacagctcca
tcaataagaactacttagcttggtaccagcagaaaccaggacagcctcct
aagctgctcatttactgggcatctacccgggaatccggggtccctgaccg
attcagtggcagcgggtctgggacagatttcactctcaccatcagcagcc
tgcaggctgaagatgtggcagtttattactgtcagcaatattatagtact
ccgtacacttttggccaggggaccaaggtggaaatcaaa
Nucleic acid sequence of CR3022_HC
(SEQ ID NO. 29):
cagatgcagctggtgcaatctggaacagaggtgaaaaagccgggggagtc
tctgaagatctcctgtaagggttctggatacggctttatcacctactgga
tcggctgggtgcgccagatgcccgggaaaggcctggagtggatggggatc
atctatcctggtgactctgaaaccagatacagcccgtccttccaaggcca
ggtcaccatctcagccgacaagtccatcaacaccgcctacctgcagtgga
gcagcctgaaggcctcggacaccgccatatattactgtgcggggggttcg
gggatttctacccctatggacgtctggggccaagggaccacggtcaccgt
c

### Example 7: HUMIRA® 2A Peptide Construct

HUMIRA© (Adalimumab) is a disease-modifying anti-rheumatic drug and monoclonal antibody that works by inactivating tumor necrosis factor-alpha (TNFα) that can lower the ability of your immune system to fight infections. HUMIRA© is a prescription medicine used to reduce the signs and symptoms of moderate to severe rheumatoid arthritis (RA) in adults, moderate to severe polyarticular juvenile idiopathic arthritis (JIA) in children 2 years of age and older, psoriatic arthritis (PsA) in adults, moderate to severe Crohn's disease (CD) and to achieve and maintain clinical remission in adults who have not responded well to certain other medications. moderate to severe Crohn's disease (CD) and to achieve and maintain clinical remission in children 6 years of age and older when certain other treatments have not worked well enough, moderate to severe hidradenitis suppurativa (HS) in people 12 years and older.

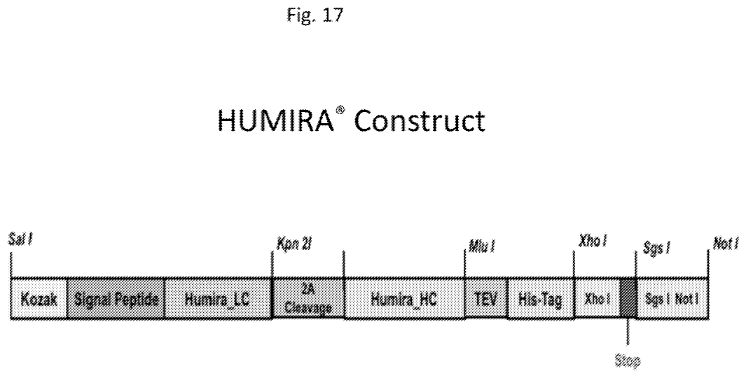

Nucleic acids of HUMIRA© heavy (HC) and light chain (LC) monoclonal antibodies are fused as a single construct with 2A cleavage peptide to facilitate efficient cleavage after secretion (FIG. 17). This fusion construct can be cloned, e.g., into a tricassette containing GAGMS2 and Egress1 with the 12× stem loop for making virus-like particles containing mRNA of the HUMIRA© HC and LC (see construct illustrated at FIG. 3).

HUMIRA ® Heavy Chain Sequence
(SEQ ID NO. 30):
GAGGTGCAGCTGGTTGAATCTGGCGGAGGACTGGTGCAGCCTGGCAGATC
cCTGAGACTGTCTTGTGCCGCCAGCGGCTTCACCTTCGACGATTATGCCA
TGCACTGGGTCCGACAGGCCCCTGGCAAAGGACTTGAATGGGTGTCCGCC
ATCACCTGGAACAGCGGCCACATCGATTACGCCGATAGCGTGGAAGGCCG
GTTCACCATCAGCAGAGACAACGCCAAGAACAGCCTGTACCTGCAGATGA
ACTCCCTGAGAGCCGAGGACACCGCCGTGTACTACTGTGCCAAGGTGTCC
TACCTGAGCACCGCCAGCAGCCTGGATTATTGGGGACAGGGCACACTGGT
CACCGTGTCTAGCGCCTCTACAAAGGGCCCTAGCGTGTTCCCTCTGGCTC
CTAGCAGCAAGTCTACAAGCGGAGGAACAGCCGCTCTGGGCTGCCTGGTC
AAGGATTACTTTCCCGAGCCTGTGACCGTGTCCTGGAACTCTGGCGCTCT
GACAAGCGGCGTGCACACCTTTCCAGCTGTGCTGCAAAGCAGCGGCCTGT
ACTCTCTGAGCAGCGTGGTCACAGTGCCTAGCTCTAGCCTGGGCACCCAG
ACCTACATCTGCAATGTGAACCACAAGCCTAGCAACACCAAGGTGGACAA
GAAGGTGGAACCCAAGAGCTGCGACAAGACCCACACCTGTCCTCCATGTC
CTGCTCCAGAACTGCTCGGCGGACCTTCCGTGTTCCTGTTTCCTCCAAAG
CCTAAGGACACCCTGATGATCAGCAGAACCCCTGAAGTGACCTGCGTGGT
GGTGGATGTGTCCCACGAaGATCCCGAAGTGAAGTTCAAcTGGTACGTGG
ACGGCGTGGAAGTGCACAATGCCAAGACCAAGCCTAGAGAGGAACAGTAC
AACAGCACCTACAGAGTGGTGTCCGTGCTGACCGTGCTGCATCAGGATTG
GCTGAACGGCAAAGAGTACAAGTGCAAAGTGTCCAACAAGGCCCTGCCTG
CTCCTATCGAGAAAACCATCTCCAAGGCCAAGGGCCAGCCTAGaGAACCC
CAGGTTTACACACTGCCTCCAAGCAGGGACGAGCTGACCAAGAATCAGGT
GTCCCTGACCTGCCTCGTGAAGGGCTTCTACCCTTCCGATATCGCCGTGG
AATGGGAGAGCAATGGCCAGCCTGAGAACAACTACAAGACAACCCCTCCT
GTGCTGGACAGCGACGGCTCATTCTTCCTGTACAGCAAGCTGACAGTGGA
CAAGTCCAGATGGCAGCAGGGCAACGTGTTCAGCTGCAGCGTGATGCACG
AGGCCCTGCACAACCACTACACCCAGAAGTCCCTGAGCCTGTCTCCTGGC
AAAtaa
HUMIRA ® Light Chain Sequence
(SEQ ID NO. 31):
GACATCCAGATGACACAGAGCCCTAGCAGCCTGTCTGCCAGCGTGGGAGA
CAGAGTGACCATCACCTGTAGAGCCAGCCAGGGCATCAGAAACTACCTGG
CCTGGTATCAGCAGAAGCCCGGCAAGGCTCCTAAGCTGCTGATCTACGCC
GCCAGCACACTGCAGTCTGGCGTGCCATCTAGATTTTCCGGCAGCGGCTC
TGGCACCGACTTCACCCTGACCATATCTAGCCTGCAGCCTGAGGACGTGG
CCACCTACTACTGCCAGAGATACAACAGAGCCCCTTACACCTTCGGCCAG
GGCACCAAGGTGGAAATCAAGAGAACAGTGGCCGCTCCTAGCGTGTTCAT
CTTCCCACCTTCCGACGAGCAGCTGAAAAGCGGCACAGCCTCTGTCGTGT
GCCTGCTGAACAACTTCTACCCCAGAGAAGCCAAGGTGCAGTGGAAGGTG
GACAACGCTCTGCAGTCCGGCAACAGCCAAGAGAGCGTGACAGAGCAGGA
CAGCAAGGACTCCACCTACAGCCTGAGCAGCACCCTGACACTGAGCAAGG
CCGACTACGAGAAGCACAAAGTGTACGCCTGCGAAGTGACCCACCAGGGC
CTTTCTAGCCCTGTGACCAAGAGCTTCAACCGGGGCGAATGTtaa

### Example 8: Insulin Construct

Insulin is a peptide hormone produced by beta cells of the pancreatic islets; it is considered to be the main hormone of the body. Insulin regulates the metabolism of carbohydrates, fats and protein by promoting the absorption of glucose from the blood into liver, fat and skeletal muscle cells.

Nucleic Acid Construct of Insulin Peptide Hormone

Nucleic acids of human insulin peptide hormone carrying the signal peptide to facilitate secretion of the peptide hormone outside of the cells that is effectuated with a fusion construct can be cloned e.g., into the disclosed tricassette containing GAGMS2 and Egress1 with the 12× stem loop for making virus-like particles containing mRNA of the insulin peptide (see FIG. 3).

Insulin nucleic acid sequence
(SEQ ID NO. 32)
Atggccctgtggatgcgcctcctgcccctgctggcgctgctggccctctg
gggacctgacccagccgcagcctttgtgaaccaacacctgtgcggctcac
acctggtggaagctctctacctagtgtgcggggaacgaggcttcttctac
acacccaagacccgccgggaggcagaggacctgcaggtggggcaggtgga
gctggggggggccctggtgcaggcagcctgcagcccttggccctggaggg
gtccctgcagaagcgtggcattgtggaacaatgctgtaccagcatctgct
ccctctaccagctggagaactactgcaactag

### Example 9: Serum Antibody Induction in Mice Following Oral Administration

This Example demonstrates that oral administration of recombinant yeast cells of the present disclosure results in serum antibody induction in mammals as exemplified in mice.

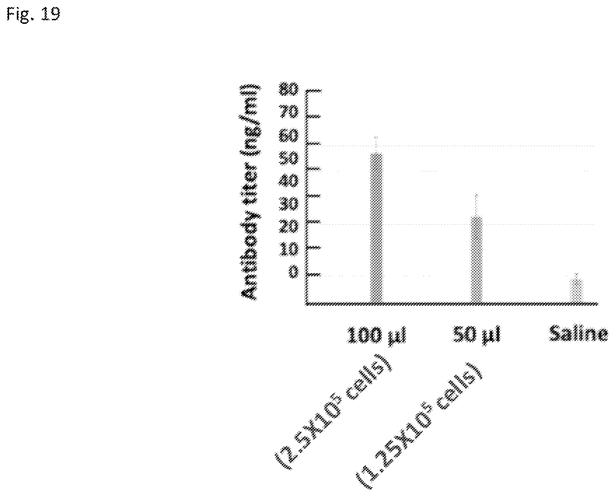

In a first set of experiments, mice were orally administered recombinant yeast cells engineered to express a GAG-GFP fusion and a cell-wall permeabilizing agent. Conditions tested included two different doses of the recombinant yeast cells, one including 2.5×10⁵ cells, and another including 1.25×10⁵ cells. Oral administration of saline served as a control. Serum antibody levels were examined 14 days following oral administration. As shown in FIG. 19, measured antibody titer was significantly increased in a dose-dependent fashion for mice that received the oral vaccine.

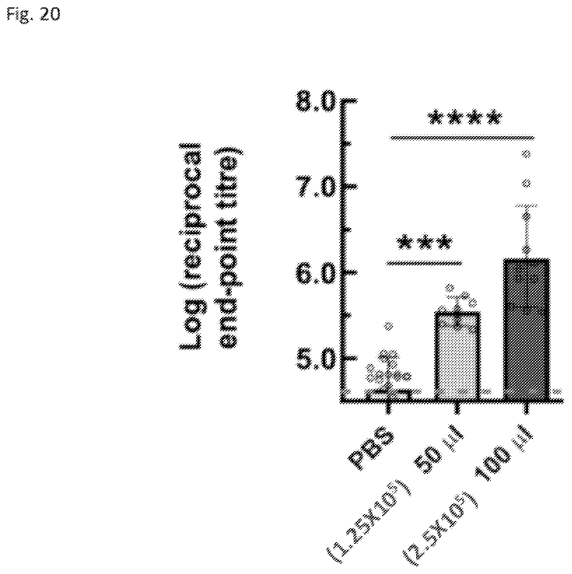

In a second set of experiments, mice were orally administered recombinant yeast cells engineered to express a GAG-MS2 fusion protein and a cell-wall permeabilizing agent, and to produce RNA transcripts encoding SARS-CoV-2 spike protein and a MS2 binding site. Conditions tested included two different doses of the recombinant yeast cells, one including 2.5×10⁵ cells, and another including 1.25×10⁵ cells. Oral administration of saline served as a control. Serum IgG levels were examined 14 days following oral administration. As shown in FIG. 20, measured antibody titer was significantly increased in a dose-dependent fashion for mice that received the oral vaccine.

Sequence information pertaining to the above disclosure is summarized below at Table 1.

TABLE 1
Sequence Information

| SEQ | | |
|---|---|---|
| ID | | |
| No. | Description | Sequence |
| 1 | β-1,3- | gaattcatga gatttccttc aatttttact gcagttttat tcgcagcatc ctccgcatta |
| | glucanase | gctgctccag tcaacactac aacagaagat gaaacggcac aaattccggc tgaagctgtc |
| | cell wall | atcggttact cagatttaga aggggatttc gatgttgctg ttttgccatt ttccaacagc |
| | degrading | acaaataacg ggttattgtt tataaatact actattgcca gcattgctgc taaagaagaa |
| | enzyme | ggggtaagct tggagaaacg cgccccagct tctgctgttc cagctaccat tccattgacc |
| | nucleotide | atcaccaacg actctggcag aggtccaatc tacctgtacg ttttgggtga gagagatggc |
| | | gttgctggtt gggctgatgc tggtggtact tttcatccat ggccaggtgg tgtgggtcca |
| | | gttccagttc ctgctccaga cgcatctatt gctggtccag gtccaggaca gtctgttacc |
| | | attagactgc caaagctgtc gggcagagtg tactactctt acggtcagaa gatgaccttc |
| | | cagatcgtgc tggacggaag attggttcag ccagctgttc agaacgactc ggacccaaac |
| | | agaaacatcc tgttcaactg gaccgagtac accttgaacg atggtggtct gtggatcaac |
| | | tctacccaag ttgaccattg gtctgcccca taccaggttg gtgttcaacg cgctgatggt |
| | | caggttttgt ctaccggtat gctgaagcca aacggttacg aggctttcta caccgctttg |
| | | gaatctgctg gatggggtgg tttggttcaa agagcaccag acggttctag actgagagca |
| | | ttgaacccat ctcacggtat cgacgtgggc aagatttctt ctgcctcgat cgactcttac |
| | | gtgaccgagg tttggaactc gtacagaacc agagacatgt gcgtgacccc attttctcac |
| | | gagccaggta ctcagttcag aggtagagtg gatggtgact ggttcagatt cagaaacggc |
| | | tctggtcaag aggttgccgc ttttaagaaa ccagacgcct cttctgtgta cggctgtcac |
| | | aaagatcttc aggctccaaa cgaccacgtt gtgggaccaa ttgctagaac cttgtgtgcc |
| | | gctctggtta gaaccactgc tctgactaac ccaaaccagc cagatgctaa ctctgccggt |
| | | ttttaccagg acgccagaac taacgtttac gccaaattgg ctcaccagca gatggccaac |
| | | ggtaaggctt acgcttttgc tttcgacgac gttggtgctc acgagtcttt ggttcatgac |
| | | ggtaatccac aggccgccta cattaagttg gacccattca ctggtactgc taccccaatt |
| | | gcaaacggtg gttctactga gcaacctgga actccaggtg gtttgccagc tggtactggt |
| | | gctttgagaa ttggttctac cctgtgtctg gacgttccat gggctgatcc aactgacact |
| | | aaccaggttc agctggctac ctgttctggt aacgctgctc aacaatggac cagaggtact |
| | | gacggtactg ttagagccct gggaaagtgt ttggacgtgg ctagatctgg aactgctgat |
| | | ggtacagccg tgtggatcta cacctgtaat ggaactggtg ctcagaagtg gacctacgac |
| | | tctgctacta aggctctgag aaacccacag tctggtaagt gcttggatgc tcaaggtggt |
| | | gctccattga gagatggtca gaaagttcag ctgtggacct gcaaccagac tgaggctcaa |
| | | agatggaccc tgtaaggatc c |
| 2 | chitinase | caattgatga gatttccttc aatttttact gcagttttat tcgcagcatc ctccgcatta |
| | cell wall | gctgctccag tcaacactac aacagaagat gaaacggcac aaattccggc tgaagctgtc |
| | degrading | atcggttact cagatttaga aggggatttc gatgttgctg ttttgccatt ttccaacagc |
| | enzyme | acaaataacg ggttattgtt tataaatact actattgcca gcattgctgc taaagaagaa |
| | nucleotide | ggggtaagct tggagaaacg caccccagtg tctaccaacg acgtgtcggt tgagaaaaga |
| | | gcctctggtt acaccaacgc cgtgtacttt acaaactggg gcatctacgg cagaaacttc |
| | | cagccacaag acttggttgc ctctgacatc acccacgtga tctacccatt catgaacttc |
| | | caggctgacg gaaccgttgt ttctggtgat gcttacgctg actaccagaa gcactactcg |
| | | gacgactctt ggaacgacgt tggtaacaac gcctacggtt gtgtgaacca gctgttcaag |
| | | ctgaagaagg ccaacagaaa cctgaaggtg atgctgtcta ttggcggttg gacttggtcg |
| | | actaacttcc catctgctgc ttctaccgac gctaacagaa agaacttcgc caagaccgcc |
| | | atcacgttca tgaaggattg gggtttcgac ggtatcgacg ttgactggga atacccagct |
| | | gacgatactc aggctaccaa catggttctg ctgctgaaag agatcagatc gcagttggac |
| | | gcttacgcag ctcaatacgc tccaggttac cacttcctgt tgtctattgc tgctcctgct |
| | | ggtccagagc attactctgc attgcacatg gctgaccttg gtcaggttct ggactacgtt |
| | | aacctgatgg cttacgacta cgctggctct tggtcgtctt actctggtca cgatgctaac |
| | | ctgttcgcta acccatcgaa cccaaactcg tcgccataca acactgacca ggccatcaag |
| | | gcctacatca acggtggtgt tccagcctct aagatcgttc tgggtatgcc aatctacggt |
| | | agatcgttcg agtccaccaa cggtattggt cagacctaca acggtatcgg ctctggttct |
| | | tgggagaacg gtatttggga ctacaaggtg ttgccaaagg ctggtgctac tgtgcagtac |
| | | gactctgttg ctcaggccta ctactcttac gactcgtcgt ctaaagagct gatctcgttt |
| | | gacaccccag acatggtgtc gaagaaggtg tcttacctga agaaccttgg ccttggcggt |
| | | tcgatgtttt gggaagcttc tgctgacaag accggctctg actctctgat tggtacttct |
| | | cacagagccc tgggttcttt ggactctact cagaacttgc tgtcgtaccc taacagccag |
| | | tacgacaaca tcagatcggg cctgaactaa agatct |
| 3 | Cell wall | MKFSFVYGLTGFLAATSSALPSEILSTGYERSALEKRGDGYLI |
| | inhibiting | MCKNCDPNTGSCDWKQNWNTCVGIGANVHWMVTGGSTDGKQGC |
| | toxin | ATIWEGSGCVGRSTTMCCPANTCCNINTGFYIRSYRRVE |
| | amino acid | |
| 4 | cell wall | gaattcatga gatttccttc aatttttact gcagttttat tcgcagcatc ctccgcatta |
| | inhibiting | gctgctccag tcaacactac aacagaagat gaaacggcac aaattccggc tgaagctgtc |
| | toxin | atcggttact cagatttaga aggggatttc gatgttgctg ttttgccatt ttccaacagc |
| | nucleic | acaaataacg ggttattgtt tataaatact actattgcca gcattgctgc taaagaagaa |
| | acid | ggggtaagct tggagaaacg cggtgacggt tacctgatca tgtgcaagaa ctgtgaccca |
| | | aacaccggtt cttgtgactg gaagcagaac tggaacacct gtgttggtat cggtgctaac |
| | | gtgcactgga tggttactgg tggttctact gacggtaagc agggttgtgc taccatctgg |
| | | gaaggttctg gttgtgtggg aagatctacc accatgtgct gtccagctaa cacttgctgc |
| | | aacatcaaca ccggcttcta catcagatcg tacagaagag tcgagtaagg atcc |
| 5 | GAG-MS2 | MGARASVLSGGELDRWEKIRLRPGGKKKYKLKHIVWASRELER |
| | fusion | FAVNPGLLETSEGCRQILGQLQPSLQTGSEELRSLYNTVATLY |
| | | CVHQRIEIKDTKEALDKIEEEQNKSKKKAQQAAADTGHSNQVS |
| | | QNYPIVQNIQGQMVHQAISPRTLNAWVKVVEEKAFSPEVIPMF |
| | | SALSEGATPQDLNTMLNTVGGHQAAMQMLKETINEEAAEWDRV |
| | | HPVHAGPIAPGQMREPRGSDIAGTTSTLQEQIGWMTHNPPIPV |
| | | GEIYKRWIILGLNKIVRMYSPTSILDIRQGPKEPFRDYVDRFY |
| | | KTLRAEQASQEVKNWMTETLLVQNANPDCKTILKALGPGATLE |
| | | EMMTACQGVGGPGHKARVLAEAMSQVTNPATIMIQKGNFRNQR |
| | | KTVKCFNCGKEGHIAKNCRAPRKKGCWKCGKEGHQMKDCTERQ |
| | | ANFLGKIWPSHKGRPGNFLQSRPEPTAPPEESFRFGEETTTPS |
| | | QKQEPIDKELYPLASLRSLFGSDPSSQGGSTSSEFCSRRYRGP |
| | | GIHRPVATMASNFTQFVLVDNGGTGDVTVAPSNFANGIAEWIS |
| | | SNSRSQAYKVTCSVRQSSAQNRKYTIKVEVPKGAWRSYLNMEL |
| | | TIPIFATNSDCELIVKAMQGLLKDGNPIPSAIAANSGIYAAA |
| 6 | IRES | CCCCTCTCCCTCCCCCCCCCCTAACGTTACTGGCCGAAGCCGC |
| | nucleic | TTGGAATAAGGCCGGTGTGCGTTTGTCTATATGTTATTTTCCA |
| | acid | CCATATTGCCGTCTTTTGGCAATGTGAGGGCCCGGAAACCTGG |
| | | CCCTGTCTTCTTGACGAGCATTCCTAGGGGTCTTTCCCCTCTC |
| | | GCCAAAGGAATGCAAGGTCTGTTGAATGTCGTGAAGGAAGCAG |
| | | TTCCTCTGGAAGCTTCTTGAAGACAAACAACGTCTGTAGCGAC |
| | | CCTTTGCAGGCAGCGGAACCCCCCACCTGGCGACAGGTGCCTC |
| | | TGCGGCCAAAAGCCACGTGTATAAGATACACCTGCAAAGGCGG |
| | | CACAACCCCAGTGCCACGTTGTGAGTTGGATAGTTGTGGAAAG |
| | | AGTCAAATGGCTCTCCTCAAGCGTATTCAACAAGGGGCTGAAG |
| | | GATGCCCAGAAGGTACCCCATTGTATGGGATCTGATCTGGGGC |
| | | CTCGGTGCACATGCTTTACATGTGTTTAGTCGAGGTTAAAAAA |
| | | ACGTCTAGGCCCCCCGAACCACGGGGACGTGGTTTTCCTTTGA |
| | | AAAACACGATGATAATATGGCCACAACC |
| 7 | β-1,3- | MPHDRKNSSRRAWAALCAAVLAVSGALVGVAAPASAVPATIPL |
| | glucanase | TITNDSGRGPIYLYVLGERDGVAGWADAGGTFHPWPGGVGPVP |
| | amino acid | VPAPDASIAGPGPGQSVTIRLPKLSGRVYYSYGQKMTFQIVLD |
| | | GRLVQPAVQNDSDPNRNILFNWTEYTLNDGGLWINSTQVDHWS |
| | | APYQVGVQRADGQVLSTGMLKPNGYEAFYTALESAGWGGLVQR |
| | | APDGSRLRALNPSHGIDVGKISSASIDSYVTEVWNSYRTRDMC |
| | | VTPFSHEPGTQFRGRVDGDWFRFRNGSGQEVAAFKKPDASSVY |
| | | GCHKDLQAPNDHVVGPIARTLCAALVRTTALTNPNQPDANSAG |
| | | FYQDARTNVYAKLAHQQMANGKAYAFAFDDVGAHESLVHDGNP |
| | | QAAYIKLDPFTGTATPIANGGSTEQPGTPGGLPAGTGALRIGS |
| | | TLCLDVPWADPTDTNQVQLATCSGNAAQQWTRGTDGTVRALGK |
| | | CLDVARSGTADGTAVWIYTCNGTGAQKWTYDSATKALRNPQSG |
| | | KCLDAQGGAPLRDGQKVQLWTCNQTEAQRWTL |
| 8 | Mannanase | AHTVSPVNPNAQQTTKTVMNWLAHLPNRTENRVLSGAFGGYSH |
| | amino acid | DTFSMAEADRIRSATGQSPAIYGCDYARGWLETANIEDSIDVS |
| | | CNGDLMSYWKNGGIPQISLHLANPAFQSGHFKTPITNDQYKKI |
| | | LDSSTAEGKRLNAMLSKIADGLQELENQGVPVLFRPLHEMNGE |
| | | WFWWGLTSYNQKDNERISLYKQLYKKIYHYMTDTRGLDHLIWV |
| | | YSPDANRDFKTDFYPGASYVDIVGLDAYFQDAYSINGYDQLTA |
| | | LNKPFAFTEVGPQTANGSFDYSLFINAIKQKYPKTIYFLAWND |
| | | EWSPAVNKGASALYHDSWTLNKGEIWNGDSLTPIVE |
| 9 | chitinase | MRFPSIFTAVLFAASSALAAPVNTTTEDETAQIPAEAVIGYSD |
| | cell wall | LEGDFDVAVLPFSNSTNNGLLFINTTIASIAAKEEGVSLEKRT |
| | degrading | PVSTNDVSVEKRASGYTNAVYFTNWGIYGRNFQPQDLVASDIT |
| | enzyme | HVIYPFMNFQADGTVVSGDAYADYQKHYSDDSWNDVGNNAYGC |
| | amino acid | VNQLFKLKKANRNLKVMLSIGGWTWSTNFPSAASTDANRKNFA |
| | | KTAITFMKDWGFDGIDVDWEYPADDTQATNMVLLLKEIRSQLD |
| | | AYAAQYAPGYHFLLSIAAPAGPEHYSALHMADLGQVLDYVNLM |
| | | AYDYAGSWSSYSGHDANLFANPSNPNSSPYNTDQAIKAYINGG |
| | | VPASKIVLGMPIYGRSFESTNGIGQTYNGIGSGSWENGIWDYK |
| | | VLPKAGATVQYDSVAQAYYSYDSSSKELISFDTPDMVSKKVSY |
| | | LKNLGLGGSMFWEASADKTGSDSLIGTSHRALGSLDSTQNLLS |
| | | YPNSQYDNIRSGLN |
| 10 | GAG MS2 | atgggtgcta gagcttctgt tttgtctggt ggtgaattgg atagatggga aaagatcaga |
| | fusion | ttaagaccag gtggtaagaa gaagtacaag ttgaaacata tcgtttgggc ctccagagaa |
| | nucleic | ttggaaagat ttgctgttaa tccaggcttg ttggaaactt ctgaaggttg cagacaaatc |
| | acid | ttgggtcaat tgcaaccatc tttacaaacc ggttctgaag aactgagatc cttgtataat |
| | | actgttgcta ccttgtactg cgtccatcaa agaattgaaa tcaaggatac caaagaggcc |
| | | ttggacaaga ttgaagaaga acagaacaag tccaagaaga aggctcaaca agctgctgct |
| | | gatactggtc attctaatca agtttctcag aactacccaa tegtccaaaa cattcaaggt |
| | | caaatggttc atcaagccat ttctccaaga actttgaatg catgggtcaa agttgtagaa |
| | | gaaaaggctt tttcacctga ggttatccca atgttttctg ctttatctga aggtgctact |
| | | ccacaagatt tgaacactat gttgaacact gttggtggtc atcaagctgc tatgcaaatg |
| | | ttgaaagaaa ccattaacga agaggctgct gaatgggata gagttcatcc agttcatgct |
| | | ggtccaattg cacctggtca aatgagagaa cctagaggtt ctgatattgc tggtactact |
| | | tctaccttgc aagaacaaat tggttggatg actcataacc caccaattcc agttggtgaa |
| | | atctacaaga gatggatcat tttgggcttg aacaagateg tcagaatgta ctctccaacc |
| | | tccattttgg atattagaca gggtccaaaa gaaccattca gagattacgt tgacagattc |
| | | tacaagacct tgagagctga acaagcttct caagaggtta agaattggat gaccgaaacc |
| | | ttgttggttc aaaatgctaa tccagattgc aagaccatct tgaaagcttt aggtccaggt |
| | | gctacattgg aagaaatgat gactgcttgt caaggtgtag gtggtccagg tcataaggct |
| | | agagttttgg ctgaagctat gtctcaagtt actaatccag ccaccattat gatccaaaag |
| | | ggcaacttta gaaaccagag aaagaccgtt aagtgcttca actgtggtaa agaaggtcat |
| | | attgctaaga actgtagagc cccaagaaaa aaaggttgtt ggaagtgcgg aaaagagggt |
| | | catcaaatga aggattgcac tgaaagacaa gctaacttct tgggtaaaat ttggccatct |
| | | cataagggta gaccaggtaa ctttttacaa tctagaccag aacctactgc tccaccagaa |
| | | gaatctttta gatttggtga agaaaccacc actecatete aaaagcaaga acctatcgac |
| | | aaagagttgt atccattggc ttcattgaga tctttgttcg gttctgatcc atcatctcaa |
| | | ggtggttcta catcttctga attctgctct agaagatata gaggtcctgg tattcataga |
| | | ccagttgcta ctatggcttc taacttcact caattcgttt tggttgataa cggtggtact |
| | | ggtgatgtta cagttgctcc atctaatttc gctaacggta ttgcagaatg gatctcctct |
| | | aattctagat cccaagctta caaggttacc tgctctgtta gacaatcttc tgctcagaat |
| | | agaaagtaca ccatcaaggt tgaagttcca aaaggtgctt ggagatctta cttgaacatg |
| | | gaattgacca ttccaatttt cgccaccaac tctgattgtg aattgatcgt taaggctatg |
| | | cagggtttgt tgaaggatgg taatccaatt ccatctgcta ttgctgctaa ttctggtatc |
| | | tatgctgctg cttaa |
| 11 | yGFP-MS2 | gtcgacatgg tgtctaaagg tgaagagttg ttcactggtg ttgttccaat tttggttgaa |
| | | ttggatggtg atgttaacgg ccataagttt tctgtttctg gtgaaggtga gggtgatgct |
| | | acttatggta aattgacttt gaagttcatc tgcaccacag gtaaattgcc agttccatgg |
| | | ccaactttgg ttactacttt gacttatggt gtccagtgct tttctagata cccagatcat |
| | | atgaagcaac acgacttttt caaatccgct atgccagaag gttacgttca agaaagaacc |
| | | atcttcttca aggatgacgg taactacaaa actagagccg aagttaagtt cgaaggtgat |
| | | accttggtta acaggattga attgaagggc atcgatttta aagaggacgg taacattttg |
| | | ggtcacaagt tggagtacaa ctacaactct cataacgttt acatcatggc cgacaagcaa |
| | | aagaacggta ttaaggttaa cttcaagatc aggcacaaca togaagatgg tagtgttcaa |
| | | ttggctgatc actaccaaca aaacactcca attggtgatg gtccagtttt gttgccagat |
| | | aaccattact tgtctaccca atctgctttg tctaaggacc caaacgaaaa aagggatcac |
| | | atggtcttgt tggaatttgt tactgctgct ggtattacct taggtatgga cgaattatac |
| | | aagtaaggat cctaaggtac ctaattgcct agaaaacatg aggatcaccc atgtctgcag |
| | | gtcgactcta gaaaacatga ggatcaccca tgtctgcagt attcccgggt tcattagatc |
| | | ctaaggtacc taattgccta gaaaacatga ggatcaccca tgtctgcagg togactctag |
| | | aaaacatgag gatcacccat gtctgcagta ttcccgggtt cattagatcc taaggtacct |
| | | aattgcctag aaaacatgag gatcacccat gtctgcaggt cgactccaga aaacatgagg |
| | | atcacccatg tctgcagtat tcccgggttc attagatcct aaggtaccta attgcctaga |
| | | aaacatgagg atcacccatg tctgcaggtc gactctagaa aacatgagga tcacccatgt |
| | | ctgcagtatt cccgggttca ttagatccta aggtacctaa ttgcctagaa aacatgagga |
| | | tcacccatgt ctgcaggtog actctagaaa acatgaggat cacccatgtc tgcagtattc |
| | | ccgggttcat tagatcctaa ggtacctaat tgcctagaaa acatgaggat cacccatgtc |
| | | tgcaggtoga ctccagaaaa catgaggatc acccatgtct gcagtattcc cgggttcatt |
| | | agatctgcgg ccgc |
| 12 | MS2 | NRNDSASSANCASSSNNYN |
| | hairpin | |
| | loop | |
| | forming | |
| | amino acid | |
| | sequence | |
| 13 | MS2 24X | tcgcgcgttt cggtgatgac ggtgaaaacc tctgacacat gcagctcccg gagacggtca |
| | repeat | cagcttgtct gtaagcggat gccgggagca gacaagcccg tcagggcgcg tcagcgggtg |
| | nucleic | ttggcgggtg tcggggctgg cttaactatg cggcatcaga gcagattgta ctgagagtgc |
| | acid | accatatgcg gtgtgaaata ccgcacagat gcgtaaggag aaaataccgc atcaggcgcc |
| | sequence | attcgccatt caggctgcgc aactgttggg aagggcgatc ggtgcgggcc tcttcgctat |
| | | tacgccagct ggcgaaaggg ggatgtgctg caaggcgatt aagttgggta acgccagggt |
| | | tttcccagtc acgacgttgt aaaacgacgg ccagtgaatt cgagctcggt acctcgcgaa |
| | | tgcatctaga tcccaatacc ggtaacctac aaacgggtgg aggatcaccc cacccgacac |
| | | ttcacaatca aggggtacaa tacacaaggg tggaggaaca ccccaccctc cagacacatt |
| | | acacagaaat ccaatcaaac agaagcacca tcagggcttc tgctaccaaa tttatctcaa |
| | | aaaactacaa caaggaatca ccatcaggga ttccctgtgc aatatacgtc aaacgagggc |
| | | cacgacggga ggacgatcac gcctcccgaa tatcggcatg tctggctttc gaattcagtg |
| | | cgtggagcat cagcccacgc agccaatcag agtcgaatac aagtcgactt tcgcgaagag |
| | | catcagcctt cgcgccattc ttacacaaac cacactctcc cctacaggaa cagcatcagc |
| | | gttcctgccc agtacccaac tcaagaaaat ttatgtcccc atgcagcatc agcgcatggg |
| | | ccccaagaat acatccccaa caaaatcaca tccgagcacc aacagggctc ggagtgttgt |
| | | ttcttgtcca actggacaaa ccctccatgg accatcaggc catggactct caccaacaag |
| | | acaaaaacta ctcttctcga agcagcatca gcgcttcgaa acactcgagc atacattgtg |
| | | cctatttctt gggtggacga tcacgccacc catgctctca cgaatttcaa aacacggaca |
| | | aggacgagca ccaccagggc tcgtcgttcc acgtccaata cgattactta cctttcggga |
| | | tcacgatcac ggatcccgca gctacatcac ttccactcag gacattcaag catgcacgat |
| | | cacggcatgc tccacaagtc tcaaccacag aaactaccaa atgggttcag caccagcgaa |
| | | cccactccta cctcaaacct cttcccacaa aactggcaag caggatcacc gcttgcccat |
| | | tccaacatac caaatcaaaa acaattactg gtacagcatc agcgtaccag cccacatctc |
| | | tcactactat caaaaaccaa accgttcagc aacagcgaac ggtacacacg gaaaaatcaa |
| | | ctggtttaca aatacgaaag acgatcacgc tttcgtccag cgcaaactat tacgaaaaac |
| | | atccgacggg aagagcaaca gccttcccgc ggcggaaaac ctcacaaaaa cacgacaaac |
| | | ggatgcacga acacggcatc cgccgacaac ccacaaactt acaaccaggc aaacggtgca |
| | | ggatcaccgc accgtacatc aaacacctca gatctcatat cgatattggg tcatcggatc |
| | | ccgggcccgt cgactgcaga ggcctgcatg caagcttggc gtaatcatgg tcatagctgt |
| | | ttcctgtgtg aaattgttat ccgctcacaa ttccacacaa catacgagcc ggaagcataa |
| | | agtgtaaagc ctggggtgcc taatgagtga gctaactcac attaattgcg ttgcgctcac |
| | | tgcccgcttt ccagtcggga aacctgtcgt gccagctgca ttaatgaatc ggccaacgcg |
| | | cggggagagg cggtttgcgt attgggcgct cttccgcttc ctcgctcact gactcgctgc |
| | | gctcggtcgt tcggctgcgg cgagcggtat cagctcactc aaaggcggta atacggttat |
| | | ccacagaatc aggggataac gcaggaaaga acatgtgagc aaaaggccag caaaaggcca |
| | | ggaaccgtaa aaaggccgcg ttgctggcgt ttttccatag gctccgcccc cctgacgagc |
| | | atcacaaaaa tcgacgctca agtcagaggt ggcgaaaccc gacaggacta taaagatacc |
| | | aggcgtttcc ccctggaagc tccctcgtgc gctctcctgt tccgaccctg ccgcttaccg |
| | | gatacctgtc cgcctttctc ccttcgggaa gcgtggcgct ttctcatagc tcacgctgta |
| | | ggtatctcag ttcggtgtag gtcgttcgct ccaagctggg ctgtgtgcac gaaccccccg |
| | | ttcagcccga ccgctgcgcc ttatccggta actatcgtct tgagtccaac ccggtaagac |
| | | acgacttatc gccactggca gcagccactg gtaacaggat tagcagagcg aggtatgtag |
| | | gcggtgctac agagttcttg aagtggtggc ctaactacgg ctacactaga agaacagtat |
| | | ttggtatctg cgctctgctg aagccagtta ccttcggaaa aagagttggt agctcttgat |
| | | ccggcaaaca aaccaccgct ggtagcggtg gtttttttgt ttgcaagcag cagattacgc |
| | | gcagaaaaaa aggatctcaa gaagatcctt tgatcttttc tacggggtct gacgctcagt |
| | | ggaacgaaaa ctcacgttaa gggattttgg tcatgagatt atcaaaaagg atcttcacct |
| | | agatcctttt aaattaaaaa tgaagtttta aatcaatcta aagtatatat gagtaaactt |
| | | ggtctgacag ttaccaatgc ttaatcagtg aggcacctat ctcagcgatc tgtctatttc |
| | | gttcatccat agttgcctga ctccccgtcg tgtagataac tacgatacgg gagggcttac |
| | | catctggccc cagtgctgca atgataccgc gagacccacg ctcaccggct ccagatttat |
| | | cagcaataaa ccagccagcc ggaagggccg agcgcagaag tggtcctgca actttatccg |
| | | cctccatcca gtctattaat tgttgccggg aagctagagt aagtagttcg ccagttaata |
| | | gtttgcgcaa cgttgttgcc attgctacag gcatcgtggt gtcacgctcg tcgtttggta |
| | | tggcttcatt cagctccggt tcccaacgat caaggcgagt tacatgatcc cccatgttgt |
| | | gcaaaaaagc ggttagctcc ttcggtcctc cgatcgttgt cagaagtaag ttggccgcag |
| | | tgttatcact catggttatg gcagcactgc ataattctct tactgtcatg ccatccgtaa |
| | | gatgcttttc tgtgactggt gagtactcaa ccaagtcatt ctgagaatag tgtatgcggc |
| | | gaccgagttg ctcttgcccg gcgtcaatac gggataatac cgcgccacat agcagaactt |
| | | taaaagtgct catcattgga aaacgttctt cggggcgaaa actctcaagg atcttaccgc |
| | | tgttgagatc cagttcgatg taacccactc gtgcacccaa ctgatcttca gcatctttta |
| | | ctttcaccag cgtttctggg tgagcaaaaa caggaaggca aaatgccgca aaaaagggaa |
| | | taagggcgac acggaaatgt tgaatactca tactcttcct ttttcaatat tattgaagca |
| | | tttatcaggg ttattgtctc atgagcggat acatatttga atgtatttag aaaaataaac |
| | | aaataggggt tccgcgcaca tttccccgaa aagtgccacc tgacgtctaa gaaaccatta |
| | | ttatcatgac attaacctat aaaaataggc gtatcacgag gccctttcgt c |
| 14 | PIV5 | MGIIIQFLVVSCLLAGAGGLDPAALMQIGVIPTNVRQLMYYTE |
| | fusion | ASSAFIVVKLMPTIDSPISGCNITSISSYNATVTKLLQPIGEN |
| | glyco- | LETIRNQLIPTRRRRRFAGVVIGLAALGVATAAQVTAAVALVK |
| | protein | ANENAAAILNLKNAIQKTNAAVADVVQATQSLGTAVQAVQDHI |
| | amino acid | NSVVSPAITAANCKAQDAIIGSILNLYLTELTTIFHNQITNPA |
| | | LSPITIQALRILLGSTLPTVVEKSFNTQISAAELLSSGLLTGQ |
| | | IVGLDLTYMQMVIKIELPTLTVQPATQIIDLATISAFINNQEV |
| | | MAQLPTRVMVTGSLIQAYPASQCTITPNTVYCRYNDAQVLSDD |
| | | TMACLQGNLTRCTFSPVVGSFFTRFVLFDGIVYANCRSMLCKC |
| | | MQPAAVILQPSSSPVTVIDMYKCVSLQLDNLRFTITQLANVTY |
| | | NSTIKLESSQILPIDPLDISQNLAAVNKSLSDALQHLAQSDTY |
| | | LSAITSATTTSVLSIIAICLGSLGFILIILLSVVVWKLLTIVA |
| | | ANRNRMENFVYHNQAFHHSQSDLSEKNQPATLGTR |
| 15 | PIV5 L | MAGSREILLPEVHLNSPIVKHKLYYYILLGNLPNEIDLDDLGP |
| | protein | LHNQNWNQIAHEESNLAQRLVNVRNFLITHIPDLRKGHWQEYV |
| | amino acid | NVILWPRILPLIPDFKINDQLPLLKNWDKLVKESCSVINAGTS |
| | | QCIQNLSYGLTGRGNLFTRSRELSGDRRDIDLKTVVAAWHDSD |
| | | WKRISDFWIMIKFQMRQLIVRQTDHNDPDLITYIENREGIIII |
| | | TPELVALFNTENHTLTYMTFEIVLMISDMYEGRHNILSLCTVS |
| | | TYLNPLKKRITYLLSLVDNLAFQIGDAVYNIIALLESFVYAQL |
| | | QMSDPIPELRGQFHAFVCSEILDALRGTNSFTQDELRTVTTNL |
| | | ISPFQDLTPDLTAELLCIMRLWGHPMLTASQAAGKVRESMCAG |
| | | KVLDFSTIMKTLAFFHTILINGYRRKHHGVWPPLNLPGNASKG |
| | | LTELMNDNTEISYEFTLKHWKEISLIKFKKCFDADAGEELSIF |
| | | MKDKAISAPKQDWMSVFRRSLIKQRHQHHQVPLPNPFNRRLLL |
| | | NFLEDDKFDPNVELQYVTSGEYLHDDTFCASYSLKEKEIKPDG |
| | | RIFAKLTKRMRSCQVIAESLLANHAGKLMKENGVVMNQLSLTK |
| | | SLLTMSQIGIISEKARKSTRDNINQPGFQNIQRNKSHHSKQVN |
| | | QRDPSDDFELAASFLTTDLKKYCLOWRYQTIIPFAQSLNRMYG |
| | | YPHLFEWIHLRLMRSTLYVGDPFNPPADTSQFDLDKVINGDIF |
| | | IVSPRGGIEGLCQKAWTMISISVIILSATESGTRVMSMVQGDN |
| | | QAIAVTTRVPRSLPTLEKKTIAFRSCNLFFERLKCNNFGLGHH |
| | | LKEQETIISSHFFVYSKRIFYQGRILTQALKNASKLCLTADVL |
| | | GECTQSSCSNLATTVMRLTENGVEKDICFYLNIYMTIKQLSYD |
| | | IIFPQVSIPGDQITLEYINNPHLVSRLALLPSQLGGLNYLSCS |
| | | RLFNRNIGDPVVSAVADLKRLIKSGCMDYWILYNLLGRKPGNG |
| | | SWATLAADPYSINIEYQYPPTTALKRHTQQALMELSTNPMLRG |
| | | IFSDNAQAEENNLARFLLDREVVFPRVAHIIIEQTSVGRRKQI |
| | | QGYLDSTRSIMRKSLESKPLSNRKLNEILDYNINYLAYNLALL |
| | | KNAIEPPTYLKAMTLETCSIDIARSLRKLSWAPLLGGRNLEGL |
| | | ETPDPIEITAGALIVGSSYCEQCAAGDNRFTWFFLPSGIEIGG |
| | | DPRDNPPIRVPYIGSRTDERRVASMAYIRGASSSLKAVLRLAG |
| | | VYIWAFGDTLENWIDALDLSHTRVNITLEQLQSLTPLPTSANL |
| | | THRLDDGTTTLKFTPASSYTFSSFTHISNDEQYLTINDKTADS |
| | | NIIYQQLMITGLGILETWNNPPINRTFEESTLHLHTGASCCVR |
| | | PVDSCIISEALTVKPHITVPYSNKFVFDEDPLSEYETAKLESL |
| | | SFQAQLGNIDAVDMTGKLTLLSQFTARQIINAITGLDESVSLI |
| | | NDAIVASDYVSNWISECMYTKLDELFMYCGWELLLELSYQMYY |
| | | LRVVGWSNIVDYSYMILRRIPGAALNNLASTLSHPKLFRRAIN |
| | | LDIVAPLNAPHFASLDYIKMSVDAILWGCKRVINVLSNGGDLE |
| | | LVVTSEDSLILSDRSMNLIARKLTLLSLIHHNGLELPKIKGFS |
| | | PDEKCFALTEFLRKVVNSGLSSIENLSNFMYNVENPRLAAFAS |
| | | NNYYLTRKLLNSIRDTESGQVAVTSYYESLEYIDSLKLTPHVP |
| | | GTSCIEDDSLCINDYIIWIIESNANLEKYPIPNSPEDDSNFHN |
| | | FKLNAPSHHTLRPLGLSSTAWYKGISCCRYLERLKLPQGDHLY |
| | | IAEGSGASMTIIEYLFPGRKIYYNSLFSSGDNPPQRNYAPMPT |
| | | QFIESVPYKLWQAHTDQYPEIFEDFIPLWNGNAAMTDIGMTAC |
| | | VEFIINRVGPRTCSLVHVDLESSASLNQQCLSKPIINAIITAT |
| | | TVLCPHGVLILKYSWLPFTRFSTLITFLWCYFERITVLRSTYS |
| | | DPANHEVYLICILANNFAFQTVSQATGMAMTLTDQGFTLISPE |
| | | RINQYWDGHLKQERIVAEAIDKVVLGENALFNSSDNELILKCG |
| | | GTPNARNLIDIEPVATFIEFEQLICTMLTTHLKEIIDITRSGT |
| | | QDYESLLLTPYNLGLLGKISTIVRLLTERILNHTIRNWLILPP |
| | | SLRMIVKQDLEFGIFRITSIFNSDRFLKLSPNRKYLIAQLTAG |
| | | YIRKLIEGDCNIDLTRPIQKQIWKALGCVVYCHDPMDQRESTE |
| | | FIDININEEIDRGIDGEEI |
| 16 | PIV5 NP | MSSVLKAYERFTLTQELQDQSEEGTIPPTTLKPVIRVFILTSN |
| | protein | NPELRSRLLLFCLRIVLSNGARDSHRFGALLTMFSLPSATMLN |
| | amino acid | HVKLADQSPEADIERVEIDGFEEGSFRLIPNARSGMSRGEINA |
| | | YAALAEDLPDTLNHATPFVDSEVEGTAWDEIETFLDMCYSVLM |
| | | QAWIVTCKCMTAPDQPAASIEKRLQKYRQQGRINPRYLLQPEA |
| | | RRIIQNVIRKGMVVRHFLTFELQLARAQSLVSNRYYAMVGDVG |
| | | KYIENCGMGGFFLTLKYALGTRWPTLALAAFSGELTKLKSLMA |
| | | LYQTLGEQARYLALLESPHLMDFAAANYPLLYSYAMGIGYVLD |
| | | VNMRNYAFSRSYMNKTYFQLGMETARKQQGAVDMRMAEDLGLT |
| | | QAERTEMANTLAKLTTANRGADTRGGVNPFSSVTGTTQVPASA |
| | | TGDTFEGYMAADRLRQRYADAGTYDDEMPPLEEEKEDDTSAGP |
| | | RTGPTLEQVALDIQNAAVGAPIHTDDLNAALGDLDI |
| 17 | PIV5 V/P | MDPTDLSFSPDEINKLIETGLNTVEYFTSQQVTGTSSLEKNTI |
| | protein | PPGVTGLLTNAAEAKIQESTNHQKGSVGGGAKPKKPRPKIAIV |
| | amino acid | PADDKTVPGKPIPNPLLGLDSTPSTQTVLDLSGKTLPSGSYKG |
| | | VKLAKFGKENLMTRFIEEPRENPIATSFPIDFKRGAGIPAGSI |
| | | EGSTQLDGWEMKSRSLSGAIHPVLQSPLQQGDLNALVTSVQSL |
| | | ALNVNEILNTVRNLDSRMNQLETKVDRILSSQSLIQTIKNDIV |
| | | GLKAGMATLEGMITTVKIMDPGVPSNVTVEDVRKTLSNHAVVV |
| | | PESFNDSFLTQSEDVISLDELARPTATSVKKIVRKVPPQKDLT |
| | | GLKITLEQLAKDCISKPKMREEYLLKINQASSEAQLIDLKKAI |
| | | IRSAI |
| 18 | Linearized | GGTACCCACCAGAGATTGGCACCCTTCCAGACATATTTCGTTC |
| | pOCH | GCAAAGAACCATAAAGATAAAGAACCCTATTCAACATACACCT |
| | plasmid | ACCACTCTCCAAGGCCAGGCGATGATTCCACGCAAGAGGGTAT |
| | nucleic | TTTGTGGCCCGTACACTGTGTGAAAAACACCTGGGGTAGTAAT |
| | acid | TGGTTGACCAAATAATGGACCAAGTGGTCACTAAGCATATTAA |
| | | GATTGTATACAAGGGTTTCTTGACTGACCGTGAATACTACTCC |
| | | GCCTTCCACGACATCTGGAACTTCCATAAGACCGACATGAACA |
| | | AGTACTTAGAAAAGCATCATACAGACGAGGTTTACATTGTCGG |
| | | TGTAGCTTTGGAGTATTGTGTCAAAGCCACCGCCATTTCCGCT |
| | | GCAGAACTAGGTTATAAGACCACTGTCCTGCTGGATTACACAA |
| | | GACCCATCAGCGATGATCCCGAAGTCATCAATAAGGTTAAGGA |
| | | AGAGTTGAAGGCCCACAACATCAATGTCGTGGATAAATAAGAG |
| | | CTGAATAATACTTCTTCAACCTGATGAACTAGGGTGGCTTGCA |
| | | AATGCACAAATCTATATAACAATATCTATATATATGTATGTAC |
| | | ACCGAATTCCGACATATGGAGAAGGAATAATAAAATATTAACT |
| | | AACGTCTTTACGCCTCTCTTTATTCTTTTTTGGGTAAATTGCT |
| | | TAAACTATTTGGCCGGCCCACCGCGAAAAGATTTGGCTGGGCC |
| | | TCAACTAAACGCGCCTTTTTGGACTTTTCACGTTGCAGGGACA |
| | | GCAACGTCAAAACTTCTGCATTAAGGTAGTTTGGTAGCTTGGT |
| | | AGCCACTTTAGTATTTCTGCCTTCTTCGAATACCGACATTATT |
| | | TCTCGCCAATCCACATTCTCTCTCCCCATCTGCATCCTTTTAT |
| | | ATTTAATAGGGATAGGTTGTTTTAGTTCTTTGATTCCGTTTTC |
| | | ATTTCAAGAGCAATAATAGCAATTTGGAAAAAGAAAGCAAGTA |
| | | AAAGAAAGAAGAGATCTTATTTTTTGCTTTTTCTCTTGAGGTC |
| | | ACATGATCGCAAAATGGCAAATGGCACGTGAAGCTGTCGATAT |
| | | TGGGGAACTGTGGTGGTTGGCAAATGACTAATTAAGTTAGTCA |
| | | AGGCGCCATCCTCATGAAAACTGTGTAACATAATAACCGAAGT |
| | | GTCGAAAAGGTGGCACCTTGTCCAATTGAACACGCTCGATGAA |
| | | AAAAATAAGATATATATAAGGTTAAGTAAAGCGTCTGTTAGAA |
| | | AGGAAGTTTTTCCTTTTTCTTGCTCTCTTGTCTTTTCATCTAC |
| | | TATTTCCTTCGTGTAATACAGGGTCGTCAGATACATAGATACA |
| | | ATTCTATTACCCCCATCCATACACCCGCCGCCACCATGCCATC |
| | | TCATTTCGATACTGTTCAACTACACGCCGGCCAAGAGAACCCT |
| | | GGTGACAATGCTCACAGATCCAGAGCTGTACCAATTTACGCCA |
| | | CCACTTCTTATGTTTTCGAAAACTCTAAGCATGGTTCGCAATT |
| | | GTTTGGTCTAGAAGTTCCAGGTTACGTCTATTCCCGTTTCCAA |
| | | AACCCAACCAGTAATGTTTTGGAAGAAAGAATTGCTGCTTTAG |
| | | AAGGTGGTGCTGCTGCTTTGGCTGTTTCCTCCGGTCAAGCCGC |
| | | TCAAACCCTTGCCATCCAAGGTTTGGCACACACTGGTGACAAC |
| | | ATCGTTTCCACTTCTTACTTATACGGTGGTACTTATAACCAGT |
| | | TCAAAATCTCGTTCAAAAGATTTGGTATCGAGGCTAGATTTGT |
| | | TGAAGGTGACAATCCAGAAGAATTCGAAAAGGTCTTTGATGAA |
| | | AGAACCAAGGCTGTTTATTTGGAAACCATTGGTAATCCAAAGT |
| | | ACAATGTTCCGGATTTTGAAAAAATTGTTGCAATTGCTCACAA |
| | | ACACGGTATTCCAGTTGTCGTTGACAACACATTTGGTGCCGGT |
| | | GGTTACTTCTGTCAGCCAATTAAATACGGTGCTGATATTGTAA |
| | | CACATTCTGCTACCAAATGGATTGGTGGTCATGGTACTACTAT |
| | | CGGTGGTATTATTGTTGACTCTGGTAAGTTCCCATGGAAGGAC |
| | | TACCCAGAAAAGTTCCCTCAATTCTCTCAACCTGCCGAAGGAT |
| | | ATCACGGTACTATCTACAATGAAGCCTACGGTAACTTGGCATA |
| | | CATCGTTCATGTTAGAACTGAACTATTAAGAGATTTGGGTCCA |
| | | TTGATGAACCCATTTGCCTCTTTCTTGCTACTACAAGGTGTTG |
| | | AAACATTATCTTTGAGAGCTGAAAGACACGGTGAAAATGCATT |
| | | GAAGTTAGCCAAATGGTTAGAACAATCCCCATACGTATCTTGG |
| | | GTTTCATACCCTGGTTTAGCATCTCATTCTCATCATGAAAATG |
| | | CTAAGAAGTATCTATCTAACGGTTTCGGTGGTGTCTTATCTTT |
| | | CGGTGTAAAAGACTTACCAAATGCCGACAAGGAAACTGACCCA |
| | | TTCAAACTTTCTGGTGCTCAAGTTGTTGACAATTTAAAGCTTG |
| | | CCTCTAACTTGGCCAATGTTGGTGATGCCAAGACCTTAGTCAT |
| | | TGCTCCATACTTCACTACCCACAAACAATTAAATGACAAAGAA |
| | | AAGTTGGCATCTGGTGTTACCAAGGACTTAATTCGTGTCTCTG |
| | | TTGGTATCGAATTTATTGATGACATTATTGCAGACTTCCAGCA |
| | | ATCTTTTGAAACTGTTTTCGCTGGCCAAAAACCATAATAAGTG |
| | | TGCGTAATGAGTTGTAAAATTATGTATAAACCTCCATATCCAA |
| | | CTTCCAATTTAATCTTTCTTTTTTAATTTTCACTTATTTGCGA |
| | | TACAGAAAGAGGATCCAACGGAGCAGTGCGGCGCGAGGCACAT |
| | | CTGCGTTTCAGGAACGCGACCGGTGAAGACGAGGACGCACGGA |
| | | GGAGAGTCTTCCTTCGGAGGGCTGTCACCCGCTCGGCGGCTTC |
| | | TAATCCGTACTTCAATATAGCAATGAGCAGTTAAGCGTATTAC |
| | | TGAAAGTTCCAAAGAGAAGGTTTTTTTAGGCTAAGATAATGGG |
| | | GCTCTTTACATTTCCACAACATATAAGTAAGATTAGATATGGA |
| | | TATGTATATGGATATGTATATGGTGGTAATGCCATGTAATATG |
| | | ATTATTAAACTTCTTTGCGTCCATCCAAAAAAAAAGTAAGAAT |
| | | TTTTGGTCGACCCCGCCGCCACCATGTCTAATTTGTTGACTGT |
| | | TCATCAAAATTTGCCAGCTTTGCCAGTTGATGCAACGTCTGAT |
| | | GAGGTAAGAAAGAATTTGATGGATATGTTTAGAGATAGACAAG |
| | | CATTCTCTGAACATACTTGGAAAATGTTGTTGTCTGTTTGTAG |
| | | ATCTTGGGCTGCTTGGTGTAAATTAAATAATAGAAAATGGTTT |
| | | CCAGCTGAACCAGAAGATGTTAGAGATTATTTGTTGTATTTGC |
| | | AAGCTAGAGGTTTGGCTGTTAAAACTATACAACAACATTTGGG |
| | | TCAATTAAATATGTTGCATAGGAGATCTGGTTTGCCTAGACCA |
| | | TCTGATTCTAATGCTGTATCTTTGGTTATGAGAAGGATTAGAA |
| | | AAGAAAATGTTGATGCAGGTGAAAGAGCTAAACAAGCATTGGC |
| | | GTTTGAAAGAACTGATTTTGATCAAGTTAGATCTTTGATGGAA |
| | | AATTCTGATAGATGTCAAGATATAAGAAACTTGGCTTTCTTGG |
| | | GAATTGCTTATAATACATTGTTGAGAATTGCTGAAATTGCTAG |
| | | AATTAGAGTTAAAGACATTTCTAGAACTGATGGTGGTAGAATG |
| | | TTGATTCATATTGGTAGAACTAAAACATTGGTTTCAACTGCTG |
| | | GCGTTGAGAAAGCATTGTCTTTGGGTGTTACTAAATTGGTTGA |
| | | AAGATGGATTTCTGTATCTGGTGTGGCTGATGACCCAAATAAT |
| | | TATTTATTTTGTAGAGTTAGAAAGAATGGTGTTGCGGCTCCAT |
| | | CTGCTACATCTCAGCTCTCTACCCGTGCCTTGGAAGGAATTTT |
| | | TGAAGCAACTCATAGATTGATTTATGGTGCTAAAGATGATTCT |
| | | GGTCAAAGATATTTAGCATGGTCTGGTCATTCTGCTAGAGTTG |
| | | GTGCCGCTAGAGATATGGCTAGAGCTGGGGTATCTATTCCTGA |
| | | AATTATGCAAGCTGGTGGTTGGACTAATGTTAACATCGTCATG |
| | | AACTATATTAGAAATTTGGATTCTGAAACTGGTGCAATGGTTA |
| | | GACTCTTGGAAGATGGTGATTAATAAGACCGCGTCATGTAATT |
| | | AGTTATGTCACGCTTACATTCACGCCCTCCCCCCACATCCGCT |
| | | CTAACCGAAAAGGAAGGAGTTAGACAACCTGAAGTCTAGGTCC |
| | | CTATTTATTTTTTTATAGTTATGTTAGTATTAAGAACGTTATT |
| | | TATATTTCAAATTTTTCTTTTTTTTCTGTACAGACGCGTGTAC |
| | | GCATGTAACATTATACTGAAAACCTTGCTTGAGAAGGTTTTGG |
| | | GACGCTCGAAGGCTTTAATTTGCGAATTCCGAGGCTCTCCTTT |
| | | CATCATACCCTCTCTAAATAAAATTTATTCCTAGTTATTCCTT |
| | | TCTTCTTCATATCCTTAATCATGCATTCACTGCCATGTGCAAA |
| | | TAAAGGCTCTTGTGGTAGAAACTTTTAGCTCAAACGTGGTAAA |
| | | ACTATCTCAACGTCCTTCCGTAGACAACTGGTGAGCAGTGCTT |
| | | CTGTAACACTGCAGATCTCCAATGATAAATCGATACCGTACTA |
| | | ACTGCCCGCACTATAATGAACTCTTTGTATCCGTAACTATTTA |
| | | AAATATTCATTCATTTCTATAAGCTATATGTTTATATTTAGAT |
| | | TAGAGGGGTTAAAAGAAAGTTTTTCTCGAAAGCTTAATTTTAG |
| | | CTTTTACTTTGAACTTCTAGTAATTGCGAGGCAGTATCGACAA |
| | | TGCAATCTCTGAACTTATTAAATTGGAATCCTAATAAATTTTT |
| | | GGTTTTGCGGTTGTCGCACTTGCAGCAGTTTTTTGTCAAAAAG |
| | | GTTGAGCCGCTACCAGGTTCGCCAGTTGCTATCTTGCCTTTTA |
| | | ACTGTGGAAATTCCTCATTCAAGATATCCAGCGCTTCTTGAGA |
| | | GCAAAACATATCTTCACATAAGAATAGTCTTTGGCCAGCGCAT |
| | | TCGGGTTTCTCAAATGCAAGTAAATGAGCTTTTGAAACATCGC |
| | | GAACGTCAATAAAAGGACCACTGTAATTATAAAAATTGTCGCC |
| | | TAATTTATAACTAACCAAATTGGCAATAATGGCTGAAGAGCTA |
| | | TTTATTCCATTTCTAAGAGAGTCGGCAAATAGCTGAGGGCCAA |
| | | AAACAAATCCTGGGTTGATGGTTGATAGCGTAAATTTGATGCT |
| | | TGATTGGTTTTCCTCGAGAAAATCCCAAGCAGTTTTTTCAGCA |
| | | AATTTCTTGGAACCACAGTATGCGGAAACCGCGTTAGCTTGAC |
| | | AACTTTCCCAAGTATCTTTGTTCCAACTTTCCTCATTGACAAC |
| | | GAAACTAGTGTCCTTCATATCTCCGGGAGATGCAAGGGCAGGT |
| | | ACC |
| 19 | Amp-F | TTATCCGCCTCCATCCAGTC |
| | primer | |
| | nucleotide | |
| 20 | Amp-R | CTGCGGCCAACTTACTTCTG |
| | primer | |
| | nucleotide | |
| 21 | Cre-F | ccagctttgccagttgatgcaac |
| | primer | |
| | nucleotide | |
| 22 | Cre-R | gctctagccatatctctagcggcac |
| | primer | |
| | nucleotide | |
| 23 | OCHMET- | ggctgggcctcaactaaacg |
| | F primer | |
| | nucleotide | |
| 24 | OCHMET- | cgatgttgtcaccagtgtgtgcc |
| | R primer | |
| | nucleotide | |
| 25 | OCHCRE- | gtgccgctagagatatggctagagc |
| | F primer | |
| | nucleotide | |
| 26 | OCHCRE- | ctgctgcaagtgcgacaacc |
| | R primer | |
| | nucleotide | |
| 27 | SARS | caggtgcagc tgcaggagtc tgggggagga ttggtgcagg ctgggggctc tctgagactc |
| | VHH-72 | tcctgtgccg cctctggacg caccttcagt gaatatgcca tgggctggtt ccgccaggct |
| | nucleotide | ccagggaagg aacgtgagtt tgtagcaact attagctgga gtggtggtag cacatactat |
| | sequence | acagactccg tgaagggccg attcaccate tocagagaca acgccaagaa cacggtgtat |
| | | ctgcaaatga acagcctgaa acctgatgac acggccgttt attactgtgc agcagccggg |
| | | ttaggtacgg tagtatcgga gtgggattat gactatgact actggggcca ggggacccag |
| | | gtcaccgtct cctca |
| 28 | CR3022 | gacatccagttgacccagtctccagactccctggctgtgtctc |
| | Light | tgggcgagagggccaccatcaactgcaagtccagccagagtgt |
| | Chain | tttatacagctccatcaataagaactacttagcttggtaccag |
| | nucleotide | cagaaaccaggacagcctcctaagctgctcatttactgggcat |
| | | ctacccgggaatccggggtccctgaccgattcagtggcagcgg |
| | | gtctgggacagatttcactctcaccatcagcagcctgcaggct |
| | | gaagatgtggcagtttattactgtcagcaatattatagtactc |
| | | cgtacacttttggccaggggaccaaggtggaaatcaaa |
| 29 | CR3022 | cagatgcagctggtgcaatctggaacagaggtgaaaaagccgg |
| | Heavy | gggagtctctgaagatctcctgtaagggttctggatacggctt |
| | Chain | tatcacctactggatcggctgggtgcgccagatgcccgggaaa |
| | nucleotide | ggcctggagtggatggggatcatctatcctggtgactctgaaa |
| | | ccagatacagcccgtccttccaaggccaggtcaccatctcagc |
| | | cgacaagtccatcaacaccgcctacctgcagtggagcagcctg |
| | | aaggcctcggacaccgccatatattactgtgcggggggttcgg |
| | | ggatttctacccctatggacgtctggggccaagggaccacggt |
| | | caccgtc |
| 30 | HUMIRA ® | GAGGTGCAGCTGGTTGAATCTGGCGGAGGACTGGTGCAGCCTG |
| | Heavy | GCAGATCcCTGAGACTGTCTTGTGCCGCCAGCGGCTTCACCTT |
| | Chain | CGACGATTATGCCATGCACTGGGTCCGACAGGCCCCTGGCAAA |
| | nucleotide | GGACTTGAATGGGTGTCCGCCATCACCTGGAACAGCGGCCACA |
| | | TCGATTACGCCGATAGCGTGGAAGGCCGGTTCACCATCAGCAG |
| | | AGACAACGCCAAGAACAGCCTGTACCTGCAGATGAACTCCCTG |
| | | AGAGCCGAGGACACCGCCGTGTACTACTGTGCCAAGGTGTCCT |
| | | ACCTGAGCACCGCCAGCAGCCTGGATTATTGGGGACAGGGCAC |
| | | ACTGGTCACCGTGTCTAGCGCCTCTACAAAGGGCCCTAGCGTG |
| | | TTCCCTCTGGCTCCTAGCAGCAAGTCTACAAGCGGAGGAACAG |
| | | CCGCTCTGGGCTGCCTGGTCAAGGATTACTTTCCCGAGCCTGT |
| | | GACCGTGTCCTGGAAcTCTGGCGCTCTGACAAGCGGCGTGCAC |
| | | ACCTTTCCAGCTGTGCTGCAAAGCAGCGGCCTGTACTCTCTGA |
| | | GCAGCGTGGTCACAGTGCCTAGCTCTAGCCTGGGCACCCAGAC |
| | | CTACATCTGCAATGTGAACCACAAGCCTAGCAACACCAAGGTG |
| | | GACAAGAAGGTGGAACCCAAGAGCTGCGACAAGACCCACACCT |
| | | GTCCTCCATGTCCTGCTCCAGAACTGCTCGGCGGACCTTCCGT |
| | | GTTCCTGTTTCCTCCAAAGCCTAAGGACACCCTGATGATCAGC |
| | | AGAACCCCTGAAGTGACCTGCGTGGTGGTGGATGTGTCCCACG |
| | | AaGATCCCGAAGTGAAGTTCAACTGGTACGTGGACGGCGTGGA |
| | | AGTGCACAATGCCAAGACCAAGCCTAGAGAGGAACAGTACAAC |
| | | AGCACCTACAGAGTGGTGTCCGTGCTGACCGTGCTGCATCAGG |
| | | ATTGGCTGAACGGCAAAGAGTACAAGTGCAAAGTGTCCAACAA |
| | | GGCCCTGCCTGCTCCTATCGAGAAAACCATCTCCAAGGCCAAG |
| | | GGCCAGCCTAGaGAACCCCAGGTTTACACACTGCCTCCAAGCA |
| | | GGGACGAGCTGACCAAGAATCAGGTGTCCCTGACCTGCCTCGT |
| | | GAAGGGCTTCTACCCTTCCGATATCGCCGTGGAATGGGAGAGC |
| | | AATGGCCAGCCTGAGAACAACTACAAGACAACCCCTCCTGTGC |
| | | TGGACAGCGACGGCTCATTCTTCCTGTACAGCAAGCTGACAGT |
| | | GGACAAGTCCAGATGGCAGCAGGGCAACGTGTTCAGCTGCAGC |
| | | GTGATGCACGAGGCCCTGCACAACCACTACACCCAGAAGTCCC |
| | | TGAGCCTGTCTCCTGGCAAAtaa |
| 31 | HUMIRA ® | GACATCCAGATGACACAGAGCCCTAGCAGCCTGTCTGCCAGCG |
| | Light | TGGGAGACAGAGTGACCATCACCTGTAGAGCCAGCCAGGGCAT |
| | Chain | CAGAAACTACCTGGCCTGGTATCAGCAGAAGCCCGGCAAGGCT |
| | nucleotide | CCTAAGCTGCTGATCTACGCCGCCAGCACACTGCAGTCTGGCG |
| | | TGCCATCTAGATTTTCCGGCAGCGGCTCTGGCACCGACTTCAC |
| | | CCTGACCATATCTAGCCTGCAGCCTGAGGACGTGGCCACCTAC |
| | | TACTGCCAGAGATACAACAGAGCCCCTTACACCTTCGGCCAGG |
| | | GCACCAAGGTGGAAATCAAGAGAACAGTGGCCGCTCCTAGCGT |
| | | GTTCATCTTCCCACCTTCCGACGAGCAGCTGAAAAGCGGCACA |
| | | GCCTCTGTCGTGTGCCTGCTGAACAACTTCTACCCCAGAGAAG |
| | | CCAAGGTGCAGTGGAAGGTGGACAACGCTCTGCAGTCCGGCAA |
| | | CAGCCAAGAGAGCGTGACAGAGCAGGACAGCAAGGACTCCACC |
| | | TACAGCCTGAGCAGCACCCTGACACTGAGCAAGGCCGACTACG |
| | | AGAAGCACAAAGTGTACGCCTGCGAAGTGACCCACCAGGGCCT |
| | | TTCTAGCCCTGTGACCAAGAGCTTCAACCGGGGCGAATGTtaa |
| 32 | Insulin | atggccctgtggatgcgcctcctgcccctgctggcgctgctgg |
| | nucleotide | ccctctggggacctgacccagccgcagcctttgtgaaccaaca |
| | | cctgtgcggctcacacctggtggaagctctctacctagtgtgc |
| | | ggggaacgaggcttcttctacacacccaagacccgccgggagg |
| | | cagaggacctgcaggtggggcaggtggagctggggggggccct |
| | | ggtgcaggcagcctgcagcccttggccctggaggggtccctgc |
| | | agaagcgtggcattgtggaacaatgctgtaccagcatctgctc |
| | | cctctaccagctggagaactactgcaactag |
| 33 | Flexible | STSSEFCSRRYRGPGIHRPVAT |
| | linker | |
| | amino acid | |

The foregoing detailed description refers to various example recombinant yeast suitable for use in oral vaccination, vaccine compositions, methods of administering to an animal, and related methods, kits, and nucleic acid molecules. The description and appended drawings illustrating these various examples are intended only to provide examples of the subject matter which the inventors believe to be within the scope of their invention and are not intended to limit the scope of any claim in any manner. All publications, patents, patent applications, and patent publications disclosed herein are hereby incorporated by reference in the entirety and for all purposes and to the same extent as if each disclosed publication, patent, patent application, or patent publication were specifically incorporated by reference.

We claim:

1. A recombinant yeast cell comprising:
a heterologous regulatable promoter operably linked to a nucleic acid sequence encoding a cell wall permeabilizing agent; and
a heterologous promoter operably linked to a nucleic acid sequence encoding a therapeutic protein.

2. The recombinant yeast cell of claim 1, further comprising a heterologous promoter operably linked to a nucleic acid sequence encoding a VLP-forming protein sequence, preferably wherein said VLP-forming protein sequence comprises a viral structural protein or functional fragment thereof.

3. The recombinant yeast cell of claim 2, wherein said viral structural protein comprises a capsid protein, a matrix protein, a GAG protein, a GAG-homology protein, an envelope protein, functional fragments thereof, or combinations thereof.

4. The recombinant yeast cell of claim 2 or 3, wherein the nucleic acid sequence encoding the therapeutic protein further comprises an Internal Ribosome Entry Site (IRES) element inactive in yeast; optionally wherein said IRES element is SEQ ID NO. 6.

5. The recombinant yeast cell according to any one of claims 2-4, wherein said VLP-forming protein sequence is linked to a nucleic acid binding peptide, preferably wherein said nucleic acid binding peptide comprises an MS2 peptide sequence.

6. The recombinant yeast cell according to claim 5, wherein the nucleic acid sequence encoding the therapeutic protein comprises a region encoding at least one nucleic acid binding peptide ligand sequence corresponding to said nucleic acid binding peptide and a region encoding for the therapeutic protein, preferably wherein the nucleic acid binding peptide ligand sequence comprises an MS2 ligand sequence.

7. The recombinant yeast cell of any one of claims 1 to 6, wherein the nucleic acid encoding the therapeutic protein and the nucleic acid encoding the cell wall permeabilizing agent are under common genetic control.

8. The recombinant yeast cell of any one of claims 1 to 7, wherein the cell wall permeabilizing agent is a beta-glucanase, preferably wherein the beta-glucanase is a β-1-3-glucanase.

9. The recombinant yeast cell of claim 8, wherein the β-1-3-glucanase comprises a secreted protein sequence encoded by SEQ ID NO. 1, or wherein the β-1-3-glucanase comprises at least 100 contiguous amino acids of, or all of, SEQ ID NO. 7.

10. The recombinant yeast cell of claim 8, wherein the β-1-3-glucanase is at least 95%, or at least 99%, identical to a secreted protein sequence encoded by SEQ ID NO. 1, or wherein the β-1-3-glucanase is at least 95% identical to at least 100 contiguous amino acids of, or all of, SEQ ID NO. 7.

11. The recombinant yeast cell of claim 8, wherein the β-1-3-glucanase comprises no more than 1, 2, 3, 4, or 5 single amino acid substitutions, deletions, and/or additions relative to the protein encoded by SEQ ID NO. 1 or the polypeptide sequence set forth in SEQ ID NO. 7.

12. The recombinant yeast cell of any one of claims 1 to 7, wherein the cell wall permeabilizing agent is a chitinase.

13. The recombinant yeast cell of claim 12, wherein the chitinase comprises a secreted protein sequence encoded by SEQ ID NO. 2, or wherein the chitinase comprises at least 100 contiguous amino acids of, or all of, SEQ ID NO. 9.

14. The recombinant yeast cell of claim 12, wherein the chitinase is at least 95%, or at least 99%, identical to a secreted protein sequence encoded by SEQ ID NO. 2, or wherein the chitinase is at least 95% identical to at least 100 contiguous amino acids of, or all of, SEQ ID NO. 9.

15. The recombinant yeast cell of claim 12, wherein the chitinase comprises no more than 1, 2, 3, 4, or 5 single amino acid substitutions, deletions, and/or additions relative to the protein sequence encoded by SEQ ID NO. 2 or the polypeptide sequence set forth in SEQ ID NO. 9.

16. The recombinant yeast cell of any one of claims 1 to 7, wherein the cell wall permeabilizing agent is a cell wall inhibiting toxin, e.g., encoded by SEQ ID NO. 4, comprises a protein sequence encoded by SEQ ID NO. 4 or set forth in SEQ ID NO. 3, is at least 95%, or at least 99%, identical to a secreted protein sequence encoded by SEQ ID NO. 4 or set forth in SEQ ID NO. 3, or comprises no more than 1, 2, 3, 4, or 5 single amino acid substitutions, deletions, and/or additions relative to the protein sequence encoded by SEQ ID NO. 4 or set forth in SEQ ID NO. 3.

17. A method for producing a pharmaceutical composition, the method comprising:
culturing a recombinant yeast cell according to any one of claims 1 to 16 in a culture medium under conditions where the regulated promoter represses expression of the operably linked nucleic acid sequence; and
inducing expression of the nucleic acid operably linked to the heterologous regulatable promoter, thereby permeabilizing the recombinant yeast cell.

18. The method of claim 17, wherein the method comprises inducing expression of the nucleic acid operably linked to the heterologous regulatable promoter for at least a portion of the culturing and:
harvesting the permeabilized recombinant yeast cell from the culture medium; or
harvesting the therapeutic protein the culture medium.

19. The method of any one of claims 17 to 18, wherein the method comprises harvesting the recombinant yeast cell, permeabilized recombinant yeast cell, or therapeutic protein and forming a vaccine composition therefrom.

20. The method of claim 18, wherein the method comprises freeze drying harvested recombinant yeast cell or permeabilized recombinant yeast cell and forming the pharmaceutical composition therefrom.

21. The method of any one of claims 18 to 20, wherein the method comprises admixing a foodstuff with the pharmaceutical composition.

22. A method for making a pharmaceutical composition comprising a recombinant yeast cell, the method comprising:
providing a recombinant yeast cell according to any one of claims 1 to 16; and
admixing the recombinant yeast cell with a pharmaceutically acceptable excipient or foodstuff.

23. A pharmaceutical composition comprising a recombinant yeast cell according to any one of claims 1 to 16 and a pharmaceutically acceptable excipient or foodstuff.
